(19) United States
(12) Patent Application Publication
(10) Pub. No.: US 2005/0107400 A1
(43) Pub. Date: May 19, 2005

(54) Use of pyrazolopyridines as therapeutic compounds

(76) Inventors: Leslie F Boyd, Durham, NC (US); Kristian Gudmundsson, Durham, NC (US); Brian A. Johns, Durham, NC (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/473,197

(22) PCT Filed: Mar. 20, 2002
(86) PCT No.: PCT/US02/08621

Related U.S. Application Data
(60) Provisional application No. 60/280,047, filed on Mar. 30, 2001.
(60) Provisional application No. 60/307,189, filed on Jul. 23, 2001.

(51) Int. Cl. A61K 31/506; A61K 31/4745

(52) U.S. Cl. 514/256; 514/303

(57) ABSTRACT

The present invention provides methods for the treatment or prophylaxis of viral infections such as herpes viral infections.

### BACKGROUND OF THE INVENTION

The present invention relates to the use of certain pyrazolopyridine compounds in therapy. More particularly, the present invention relates to the use of these compounds compounds for the prophylaxis and treatment of herpes viral infections.

Of the DNA viruses, those of the herpes group are the sources of the most common viral illnesses in man. The group includes herpes simplex virus types 1 and 2 (HSV), varicella zoster virus (VZV), cytomegalovirus (CMV), Epstein-Barr virus (EBV), human herpes virus type 6 (HHV-6), human herpes virus type 7 (HHV-7) and human herpes virus type 8 (HHV-8). HSV-1 and HSV-2 are some of the most common infectious agents of man. Most of these viruses are able to persist in the host's neural cells; once infected, individuals are at risk of recurrent clinical manifestations of infection which can be both physically and psychologically distressing.

Herpes simplex viruses (HSV-1 and -2) are the causative agents of herpes labialis and genital herpes. HSV infection is often characterised by extensive and debilitating lesions of the skin, mouth and/or genitals. Primary infections may be subclinical although tend to be more severe than infections in individuals previously exposed to the virus. Ocular infection by HSV can lead to keratitis or cataracts thereby endangering the host's sight. Infection in the new-born, in immunocompromised patients or penetration of the infection into the central nervous system can prove fatal. In the US alone, 40 million individuals are infected with HSV-2, a number that is expected to increase to 60 million by 2007. Over 80% of individuals infected with HSV-2 are unaware they carry and spread the virus, and of those diagnosed less than 20% received oral therapies. The net result is that less than 50/% of the infected population are treated. Likewise of the 530 million individuals worldwide who carry the HSV-1, 81% of the symptomatic population remain untreated. No cure exists for HSV infection, and once infected, individuals carry the virus for life in a dormant state. Reactivation of the virus from latency occurs periodically and may be triggered by stress, environmental factors, and/or suppression of the host immune system. Currently, the use of nucleoside analogs such as valaciclovir (VALTREX®) and aciclovir (ZOVIRAX®) is the standard of care for managing genital herpes virus outbreaks.

VZV is a herpes virus which causes chickenpox and shingles. Chickenpox is the primary disease produced in a host without immunity, and in young children is usually a mild illness characterised by a vesicular rash and fever. Shingles or zoster is the recurrent form of the disease which occurs in adults who were previously infected with VZV. The clinical manifestations of shingles are characterised by neuralgia and a vesicular skin rash that is unilateral and dermatomal in distribution. Spread of inflammation may lead to paralysis or convulsions. Coma can occur if the meninges become affected. VZV is of serious concern in patients receiving immunosuppressive drugs for transplant purposes or for treatment of malignant neoplasia and is a serious complication of AIDS patients due to their impaired immune system.

In common with other herpes viruses, infection with CMV leads to a lifelong association of virus and host. Congenital infection following infection of the mother during pregnancy may give rise to clinical effects such as death or gross disease (microcephaly, hepatosplenomegaly, jaundice, mental retardation), retinitis leading to blindness or, in less severe forms, failure to thrive, and susceptibility to chest and ear infections. CMV infection in patients who are immunocompromised for example as a result of malignancy, treatment with immunosuppressive drugs following transplantation or infection with Human Immunodeficiency Virus, may give rise to retinitis, pneumonitis, gastrointestinal disorders and neurological diseases. CMV infection is also associated with cardiovascular diseases and conditions including restenosis and atheroslcerosis.

The main disease caused by EBV is acute or chronic infectious mononucleosis (glandular fever). Examples of other EBV or EBV associated diseases include lymphoproliferative disease which frequently occurs in persons with congenital or acquired cellular immune deficiency, X-linked lymphoproliferative disease which occurs namely in young boys, EBV-associated B-cell tumours, Hodgkin's disease, nasopharyngeal carcinoma, Burkitt lymphoma, non-Hodgkin lymphoma, thymomas and oral hairy leukoplakia. EBV infections have also been found in association with a variety of epithelial-cell-derived tumors of the upper and lower respiratory tracts including the lung. EBV infection has also been associated with other diseases and conditions including chronic fatigue syndrome, multiple sclerosis and Alzheimer's disease.

HHV-6 has been shown to be a causative agent of infantum subitum in children and of kidney rejection and interstitial pneumonia in kidney and bone marrow transplant patients, respectively, and may be associated with other diseases such as multiple sclerosis. There is also evidence of repression of stem cell counts in bone marrow transplant patients. HHV-7 is of undetermined disease aetiology.

Hepatitis B virus (HBV) is a viral pathogen of world-wide major importance. The virus is aetiologically associated with primary hepatocellular carcinoma and is thought to cause 80% of the world's liver cancer. Clinical effects of infection with HBV range from headache, fever, malaise, nausea, vomiting, anorexia and abdominal pains. Replication of the virus is usually controlled by the immune response, with a course of recovery lasting weeks or months in humans, but infection may be more severe leading to persistent chronic liver disease outlined above.

### BRIEF SUMMARY OF THE INVENTION

According to a first aspect of the invention there is provided a method for the prophylaxis or treatment of herpes viral infections in an animal. The method comprises administering to the animal a therapeutically effective amount of a compound of formula (I): 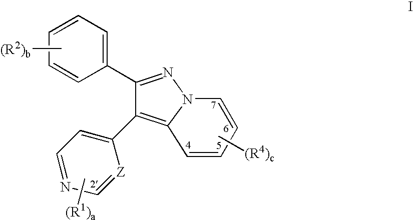
wherein:
Z is CH or N;
a is 1 or 2;
b is 1, 2 or 3;
c is 1, 2 or 3;
each R¹ is independently selected from group consisting of substituents of the formula
   —(X)_{d}—(CH₂)ₑ—R⁵
   wherein:
d is 0 or 1;
e is 0 to 6;
X is selected from the group consisting of O, NR⁶ and S(O)_{f} where f is 0, 1 or 2;
R⁵ is selected from the group consisting of H, halo, C₁₋₆alkyl, C₂₋₆alkenyl, C₂₋₆alkynyl, cycloalkyl, heterocyclyl, aryl, heteroaryl, hydroxyl, cyano, nitro, trihalomethyl, NR⁷R⁸, C₆H₄NR⁷R⁸, C₆H₄(CH₂)NR⁷R⁸, C(O)R⁷, C(O)NR⁷R⁸, OC(O)R⁷, OC(O)NR⁷R⁸, CO₂R⁷, OCO₂R⁷, SO₂R⁷, SO₂NR⁷R⁸, C(═NR⁷)NR⁷R⁸, NR⁷(C═NR⁷)NR⁷R⁸, NHC(O)R⁷ and N(C₁₋₃alkyl)C(O)R⁷;
each R² is independently selected from the group consisting of H, cyano, halo, trihalomethyl, OC₁₋₆alkyl, C₁₋₆alkyl, C₂₋₆alkenyl, C₂₋₆alkynyl, S(O)_{g}C₁₋₆alkyl where g is 0, 1 or 2, NC₁₋₆alkyl(C₁₋₆alkyl), hydroxyl and nitro;
each R⁴ is independently selected from the group consisting of substituents of the formula
   —(Y)_{d}—(CH₂)ₑ—R³
   wherein:
d is 0 or 1;
e is 0 to 6;
Y is O or S(O)_{f} where f is 0, 1 or 2;
R³ is selected from the group consisting of H, halo, C₁₋₆alkyl, C₂₋₆alkenyl, C₂₋₆alkynyl, cycloalkyl, heterocyclyl, aryl, heteroaryl, hydroxyl, cyano, nitro, trihalomethyl, phthalamido, C₆H₄NR⁷R⁸, C₆H₄(CH₂)NR⁷R⁸, C(O)R⁷, C(O)NR⁷R⁸, OC(O)R⁷, OC(O)NR⁷R⁸, CO₂R⁷, OCO₂R⁷, SO₂R⁷, SO₂NR⁷R⁸ and C(═NR⁷)NR⁷R⁸;
R⁶ is selected from the group consisting of H, C₁₋₆alkyl, C₂₋₆alkenyl, heteroaryl, cycloalkyl and heterocyclyl;
R⁷ and R⁸ are each independently selected from the group consisting of H, C₁₋₈alkyl, C₂₋₆alkenyl, SO₂C₁₋₆alkyl, (CH₂)ₘ-cycloalkyl, (CH₂)ₘ-aryl, (CH₂)ₘ-heterocyclyl and (CH₂)ₘ-heteroaryl, wherein m=0, 1 or 2,
or R⁷ and R⁸ together with the nitrogen atom to which they are bound, form a heterocyclyl group; and
wherein any of said alkyl, alkenyl and alkynyl groups may be optionally substituted with up to three substituents selected from the group consisting of halo, hydroxyl, oxo, cyano, NR⁷R⁸, C₁₋₆alkyl, OC₁₋₆alkyl, S(O)C₁₋₆alkyl, S(O)₂C₁₋₆alkyl and SO₂NR⁷R⁸; and
wherein any of said cycloalkyl, heterocyclyl, aryl and heteroaryl groups may be optionally substituted with up to three substituents selected from the group consisting of C₁₋₆alkyl, C₁₋₆alkoxy, C₁₋₆alkylsulfenyl, C₁₋₆alkylsulfinyl, C₁₋₆alkylsulfonyl, hydroxy, oxo, mercapto, nitro, cyano, halo, C₁₋₆perfluoroalkyl, amino optionally substituted by C₁₋₆alkyl, carbamoyl optionally substituted by C₁₋₆alkyl, NR⁷R⁸, carboxy and aminosulfonyl optionally substituted by C₁₋₆alkyl;
wherein when (R¹)ₐ is at the 2′ position, (R¹)ₐ is not NR⁶-aryl, NR⁶—C₆H₄NR⁷R⁸, NR⁶—C₆H₄—(CH₂)NR⁷R⁸, NR⁷R⁸ where R⁷ or R⁸ is (CH₂)ₘ-aryl and m is 0, or N-(aryl)[(C═NR⁷)NR⁷R⁸]; and
wherein when R⁴ is at the C-7 position, R⁴ is not halo, heterocyclyl, aryl, heteroaryl, phthalamido, C₆H₄NR⁷R⁸ or C₆H₄(CH₂)NR⁷R⁸;
   and pharmaceutically acceptable salts, solvates and physiologically functional derivatives thereof.

The herpes viral infection may be herpes simplex virus 1, herpes simplex virus 2, cytomegalovirus, Epstein Barr virus, varicella zoster virus, human herpes virus 6, human herpes virus 7, or human herpes virus 8.

According to a second aspect, the present invention provides a method for the prophylaxis or treatment of conditions or diseases associated with a herpes viral infection in an animal. The method comprises administering to the animal a therapeutically effective amount of the compound of formula (I).

According to a third aspect, the present invention provides the use of a compound of formula (I), for the preparation of a medicament for the prophylaxis or treatment of a herpes viral infection in an animal. The present invention also provides the use of a compound of formula (I), for the preparation of a medicament for the prophylaxis or treatment of conditions or diseases associated with a herpes viral infection in an animal.

### DETAILED DESCRIPTION OF THE INVENTION

As used herein, “a compound of the invention” or “a compound of formula (I)” means a compound of formula (I) or a pharmaceutically acceptable salt, solvate, or physiologically functional derivative thereof.

As used herein, the term “physiologically functional derivative” refers to any pharmaceutically acceptable derivative of a compound of the present invention, for example, an ester or an amide, which upon administration to a mammal, such as a human, is capable of providing (directly or indirectly) a compound of formula (I) or an active metabolite thereof. Such derivatives are clear to those skilled in the art, without undue experimentation, and with reference to the teaching of Burger's Medicinal Chemistry And Drug Discovery, 5^{th} Edition, Vol 1: Principles And Practice.

As used herein, the terms “alkyl” and “alkylene” refer to straight or branched hydrocarbon chains containing the specified number of carbon atoms. For example, C₁₋₆alkyl means a straight or branched alkyl containing at least 1, and at most 6, carbon atoms. Examples of “alkyl” as used herein include, but are not limited to, methyl, ethyl, n-propyl, n-butyl, n-pentyl, isobutyl, and isopropyl. Examples of “alkylene” as used herein include, but are not limited to, methylene, ethylene, propylene and butylene. The alkyl groups may be optionally substituted with up to three substituents selected from the group consisting of halogen, hydroxyl, oxo, cyano, NR⁷R⁸, C₁₋₆alkyl, OC₁₋₆alkyl, S(O)C₁₋₆alkyl, S(O)₂C₁₋₆alkyl and SO₂NR⁷R⁸.

As used herein, the term “alkenyl” refers to straight or branched hydrocarbon chains containing the specified number of carbon atoms and containing at least one double bond. For example, C₂₋₆alkenyl means a straight or branched alkenyl containing at least 2, and at most 6, carbon atoms and containing at least one double bond. Examples of “alkenyl” as used herein include, but are not limited to ethenyl and propenyl. The alkenyl groups may be optionally substituted with up to three substituents selected from the group consisting of halogen, hydroxyl, oxo, cyano, NR⁷R⁸, C₁₋₆alkyl, OC₁₋₆alkyl, S(O)C₁₋₆alkyl, S(O)₂C₁₋₆alkyl and SO₂NR⁷R⁸.

As used herein, the term “alkynyl” refers to straight or branched hydrocarbon chains containing the specified number of carbon atoms and containing at least one triple bond. For example, C₂₋₆alkynyl means a straight or branched alkynyl containing at least 2, and at most 6, carbon atoms and containing at least one triple bond. Examples of “alkynyl” as used herein include, but are not limited to, ethynyl and propynyl. The alkynyl groups may be optionally substituted with up to three substituents selected from the group consisting of halogen, hydroxyl, oxo, cyano, NR⁷R⁸, C₁₋₆alkyl, OC₁₋₆alkyl, S(O)C₁₋₆alkyl, S(O)₂C₁₋₆alkyl and SO₂NR⁷R⁸.

As used herein, the term “cycloalkyl” refers to a non-aromatic hydrocarbon ring having from three to twelve carbon atoms. The cycloalkyl ring may optionally contain up to three carbon-carbon double bonds. “Cycloalkyl” includes by way of example cyclopropyl, cyclobutyl, cyclopentyl, cyclohexyl, cycloheptyl and cyclooctyl. The cycloalkyl ring may be optionally substituted with substituents selected from a group consisting of C₁₋₆alkyl, C₁₋₆alkoxy, C₁₋₆alkylsulfenyl, C₁₋₆alkylsulfinyl, C₁₋₆alkylsulfonyl, hydroxy, oxo, mercapto, nitro, cyano, halogen, C₁₋₆perfluoroalkyl, amino optionally substituted by C₁₋₆alkyl, carbamoyl optionally substituted by C₁₋₆alkyl, NR⁷R⁸, carboxy, aminosulfonyl optionally substituted by C₁₋₆alkyl.

As used herein, the terms “heterocycle”, “heterocyclyl” and “heterocyclic” refer to a monocyclic five to seven membered non-aromatic hydrocarbon ring or to a fused bicyclic non-aromatic hydrocarbon ring system comprising two of such monocyclic five to seven membered non-aromatic hydrocarbon rings. The ring or rings contain at least one heteroatom selected from O, S, or N where N-oxides, sulfur oxides and sulfur dioxides are permissible heteroatom substitutions. The heterocycle ring system may optionally contain up to three carbon-carbon, or carbon-nitrogen, double bonds. The heterocycle ring system may optionally be fused to one or more benzene rings. Examples of heterocycles include, but are not limited to, tetrahydrofuran, dihydropyran, tetrahydropyran, pyran, oxetane, thietane, 1,4-dioxane, 1,3-dioxane, 1,3-dioxalane, piperidine, tetrahydropyrimidine, pyrrolidine, morpholine, thiomorpholine, thiazolidine, oxazolidine, tetrahydrothiopyran, tetrahydrothiophene, and the like. Preferred heterocycles include morpholine, piperidine, and pyrrolidine. The heterocycle ring system may be optionally substituted with substituents selected from the group consisting of C₁₋₆alkyl, C₁₋₆alkoxy, C₁₋₆alkylsulfenyl, C₁₋₆alkylsulfinyl, C₁₋₆alkylsulfonyl, hydroxy, oxo, mercapto, nitro, cyano, halogen, C₁₋₆perfluoroalkyl, amino optionally substituted by C₁₋₆alkyl, carbamoyl optionally substituted by C₁₋₆alkyl, NR⁷R⁸, carboxy and aminosulfonyl optionally substituted by C₁₋₆alkyl.

As used herein, the term “aryl” refers to an optionally substituted phenyl or naphthyl ring. The aryl rings may be optionally substituted with substituents selected from the group consisting of C₁₋₆alkyl, C₁₋₆alkoxy, C₁₋₆alkylsulfenyl, C₁₋₆alkylsulfinyl, C₁₋₆alkylsulfonyl, hydroxy, oxo, mercapto, nitro, cyano, halogen, C₁₋₆perfluoroalkyl, amino optionally substituted by C₁₋₆alkyl, carbamoyl optionally substituted by C₁₋₆alkyl, NR⁷R⁸, carboxy, and aminosulfonyl optionally substituted by C₁₋₆alkyl.

As used herein, the term “heteroaryl” refers to a monocyclic five to seven membered aromatic ring, or to a fused bicyclic aromatic ring system comprising two of such monocyclic five to seven membered aromatic rings. These heteroaryl rings contain one or more nitrogen, sulfur, or oxygen heteroatoms, where N-oxides and sulfur oxides and dioxides are permissible heteroatom substitutions. Examples of “heteroaryl” as used herein include furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, and indazole. Preferred heteroaryl groups include imidazole, pyridine and thiophene. The rings are optionally substituted with substituents selected from the group consisting of C₁₋₆alkyl, C₁₋₆alkoxy, C₁₋₆alkylsulfenyl, C₁₋₆alkylsulfinyl, C₁₋₆alkylsulfonyl, hydroxy, oxo, mercapto, nitro, cyano, halogen, C₁₋₆perfluoroalkyl, amino optionally substituted by C₁₋₆alkyl, carbamoyl optionally substituted by C₁₋₆alkyl, NR⁷R⁸, carboxy and aminosulfonyl optionally substituted by C₁₋₆alkyl.

As used herein, the term “alkoxy” refers to the group RₐO—, where Rₐ is alkyl as defined above.

As used herein, the term “alkylsulfenyl” refers to the group RₐS—, where Rₐ is alkyl as defined above.

As used herein, the term “alkylsulfinyl” refers to the group RₐS(O)—, where Rₐ is alkyl as defined above.

As used herein, the term “alkylsulfonyl” refers to the group RₐSO₂—, where Rₐ is alkyl as defined above.

As used herein, the terms “halogen” or “halo” refer to the elements fluorine, chlorine, bromine and iodine. Preferred halogens include fluorine, chlorine and bromine.

As used herein, the term “optionally” means that the subsequently described event(s) may or may not occur, and includes both event(s) which occur and events that do not occur.

As used herein, the term “substituted” refers to substitution with the named substituent or substituents, multiple degrees of substitution being allowed unless otherwise stated.

As used herein, the terms “contain” or “containing” can refer to in-line substitutions at any position along the above-defined alkyl, alkenyl, alkynyl or cycloalkyl substituents with one or more of any of O, S, SO, SO₂, N, or N-alkyl, including, for example, —CH₂—O—CH₂—, —CH₂—SO₂—CH₂—, —CH₂—NH—CH₂— and so forth.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include water, methanol, ethanol and acetic acid. Preferably the solvent used is a pharmaceutically acceptable solvent. Examples of suitable pharmaceutically acceptable solvents include water, ethanol and acetic acid. Most preferably the solvent used is water.

Certain compounds of formula (I) may exist in stereoisomeric forms (e.g. they may contain one or more asymmetric carbon atoms or may exhibit cis-trans isomerism). The individual stereoisomers (enantiomers and diastereomers) and mixtures of these are included within the scope of the present invention. The present invention also covers the individual isomers of the compounds represented by formula (I) as mixtures with isomers thereof in which one or more chiral centres are inverted. Likewise, it is understood that compounds of formula (I) may exist in tautomeric forms other than that shown in the formula and these are also included within the scope of the present invention.

Compounds of formula (I): 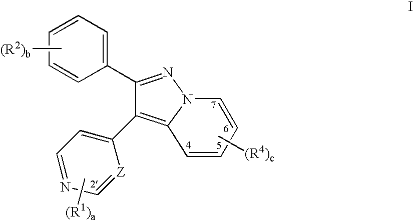
include those compounds defined wherein R¹ contains an aryl, heterocyclic or heteroaryl moiety. In one embodiment, the compounds of the present invention include those compounds defined wherein R¹ contains a heterocyclic or heteroaryl moiety.

Another class of compounds of formula (I) include those compounds defined wherein R¹ does not contain an aryl, heterocyclic or heteroaryl moiety. Another class of compounds of formula (I) include those defined wherein R¹ does not contain a heterocyclic or heteroaryl moiety but may contain an aryl moiety.

Another class of compounds of formula (I) includes those compounds defined wherein at least one R⁴ group contains an aryl, heterocyclic or heteroaryl moiety. Another class of compounds of formula (I) include those defined wherein R⁴ does not contain a heterocyclic or heteroaryl moiety but may contain an aryl moiety.

In one preferred class of compounds of formula (I), Z is CH. In another preferred class of compounds of formula (I), Z is N.

In one preferred embodiment a is 1. In another preferred embodiment, a is 1 and R¹ is located in the 2′ position of the pyridine (i.e., when Z is CH) or pyrimidine (i.e., when Z is N) ring.

R¹ is —X_{d}—(CH₂)ₑ—R⁵. In one preferred embodiment R¹ is defined where d is 1. In one preferred embodiment of R¹, X is NR⁶. When X is NR⁶, R⁶ is preferably selected from the group consisting of H, C₁₋₆alkyl, cycloalkyl, heterocyclyl and heteroaryl.

In one embodiment e is 0-3. In one preferred embodiment, R¹ is defined where e is 0.

Examples of preferred embodiments include those compounds of formula (I) where R¹ is —NR⁶—(CH₂)ₑ—R⁵. In one embodiment of such preferred compounds R⁶ is H or C₁₋₆alkyl, and R⁵ is selected from the group consisting of H, C₁₋₆alkyl, hydroxyl, NR⁷R⁸, cycloalkyl, heterocyclyl, and heteroaryl. In one preferred embodiment, R⁵ is selected from the group consisting of H, C₁₋₆alkyl, cycloalkyl, heterocyclyl and heteroaryl.

More particularly, preferred compounds of formula (I) include those defined where R¹ is selected from the group consisting of —NH₂, —NH(C₁₋₆alkyl), —NH(C₁₋₆alkyl)-OH, —NH(C₁₋₆alkyl)-NH₂, —N H(C₁₋₁-alkyl)-CF₃, —NH-heteroaryl, —NH(C₁₋₆alkyl)-heteroaryl, —NH(C₁₋₆alkyl)-aryl, —NH(C₁₋₆alkyl)-aryl(C₁₋₆alkoxy) and —NC₁₋₆alkyl(C₁₋₆alkyl).

Particular examples of preferred compounds of formula (I) include those compounds defined where R¹ is selected from the group consisting of —NH₂, —NH(CH₂)ₑCH₃, —NH(CH₂)ₑCH(CH₃)₂, —NH(CH₂)ₑOH, —NH(CH₂)ₑNH₂, —NH(CH₂)ₑCF₃,

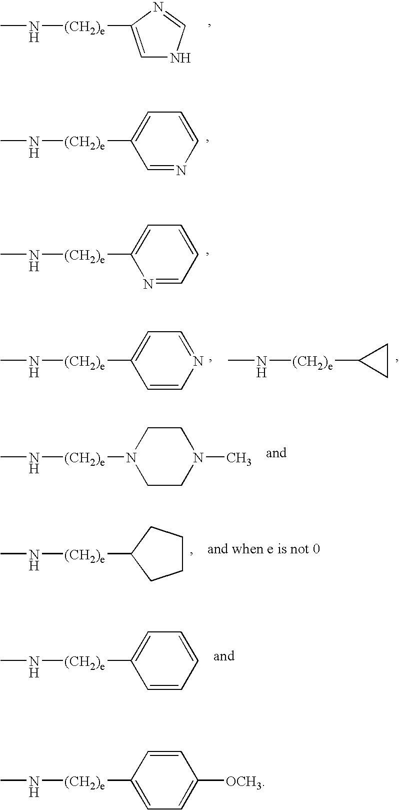

In one preferred embodiment, the compounds of formula (I) are defined wherein when (R¹)ₐ is located at the 2′ position of the pyridine or pyrimidine ring, (R¹)ₐ is not NR⁶-aryl, NR⁶—C₆H₄NR⁷R⁸, NR⁶—C₆H₄—(CH₂)NR⁷R⁸, NR⁷R⁸ where R⁷ or R⁸ is (CH₂)ₘ-aryl and m is O, or N-(aryl)[(C═NR⁷)NR⁷R⁸].

In one preferred embodiment of the present invention, the compounds of formula (I) are defined where b is 1 or 2. In another preferred embodiment b is 1.

R² is preferably selected from the group consisting of H, cyano, halo, trihalomethyl, C₁₋₆alkyl, OC₁₋₆alkyl, S(O)_{g}—C₁₋₆alkyl where g is 0, 1 or 2, N—C₁₋₆alkyl(C₁₋₆alkyl), hydroxyl and nitro. More preferably, R² is selected from the group consisting of halo (e.g., fluoro or chloro), cyano, C₁₋₆alkyl (e.g., methyl), OC₁₋₆alkyl (O-methyl, O-isobutyl, and O—CH₂cyclopropyl), N—C₁₋₃alkyl(C₁₋₃alkyl) (e.g., methylamine, dimethylamine), and hydroxyl. In one preferred embodiment R² is fluoro.

In one preferred embodiment of the present invention, the compounds of formula (I) are defined where c is 1 or 2. In another preferred embodiment c is 1. In one preferred embodiment c is 1 and R⁴ is in the 5 position. In one embodiment, the C-7 position of the pyrazolopyridine ring is unsubstituted. In another embodiment, at least one R⁴ is in the C-7 position.

R⁴ is —(Y)_{d}—(CH₂)ₑ—R³. In one preferred embodiment, R⁴ is defined where d is 0. In another preferred embodiment R⁴ is defined where d is 1.

In one preferred embodiment, R⁴ is defined where e is 0 to 3. In another preferred embodiment e is 0 or 1.

Examples of preferred embodiments include those compounds of formula (I) where R⁴ is —(CH₂)ₑ—R³ where e is 0-3 and R³ is selected from the group consisting of H, halogen, trihalomethyl, C₁₋₆alkyl, cycloalkyl, heterocyclyl, aryl, heteroaryl, hydroxyl, cyano, and C(O)NR⁷R⁸. In one preferred embodiment, R³ is heterocyclyl.

When c is 1, 2 or 3, and one R⁴ is at the C-7 position, R⁴ at the C-7 position is not a halo, a group containing an aryl ring attached directly to the pyrazolopyridine ring or a group containing a heterocyclyl or heteroaryl ring attached directly to the pyrazolopyridine ring. More particularly, when c is 1, 2 or 3 and at least one R⁴ is at the C-7 position, R⁴ at the C-7 position is not halo, heterocyclyl, aryl, heteroaryl, phthalamido, C₆H₄NR⁷R⁸ or C₆H₄(CH₂)NR⁷R⁸. In another embodiment, when R⁴ is at the C-7 position, R⁴ is not H.

R⁷ and R⁸ are preferably each independently selected from the group consisting of H, C₁₋₈alkyl, (CH₂)ₘ-cycloalkyl, (CH₂)ₘ-aryl, (CH₂)ₘ-heterocyclyl and (CH₂)ₘ-heteroaryl, where m is 0, 1 or 2.

Examples of preferred embodiments include those compounds of formula (I) where R⁴ is selected from the group consisting of H, F, Cl, Br, C₁₋₆alkyl, CF₃, CN, CH₂—NH-heterocyclyl, CH₂—OH, C(O)NH₂, and C(O)N(C₁₋₆alkyl)₂. More preferably, R⁴ is selected from the group consisting of H, F, Cl, Br, C₁₋₆alkyl, CF₃, CN, CH₂—NH-heterocyclyl and CH₂—OH.

It is to be understood that the present invention includes all combinations of the particular and preferred groups defined hereinabove.

Preferred compounds of formula (I) include but are not limited to:
4-[2-(4-Fluorophenyl)pyrazolo[1,5-a]pyridin-3-yl]-N-[2-(1H-imidazol-5-yl)ethyl]-2-pyridinamine;
N-Butyl-4-[2-(4-fluorophenyl)pyrazolo[1,5-a]pyridin-3-yl]-2-pyridinamine;
3-(4-[2-(4-Fluorophenyl)pyrazolo[1,5-a]pyridin-3-yl]-2-pyridinylamino)-1-propanol;
N¹-4-[2-(4-Fluorophenyl)pyrazolo[1,5-a]pyridin-3-yl]-2-pyridinyl-1,3-propanediamine;
4-[2-(4-Fluorophenyl)pyrazolo[1,5-a]pyridin-3-yl]-N-hexyl-2-pyridinamine;
4-[2-(4-Fluorophenyl)pyrazolo[1,5-a]pyridin-3-yl]-N-(4-methoxybenzyl)-2-pyridinamine;
4-[2-(4-Fluorophenyl)pyrazolo[1,5-a]pyridin-3-yl]-N-(3-pyridinylmethyl)-2-pyridinamine;
4-[2-(4-Fluorophenyl)pyrazolo[1,5-a]pyridin-3-yl]-N-propyl-2-pyridinamine;
2-(4-[2-(4-Fluorophenyl)pyrazolo[1,5-a]pyridin-3-yl]-2-pyridinylamino)-1-ethanol;
N-Benzyl-4-[2-(4-fluorophenyl)pyrazolo[1,5-a]pyridin-3-yl]-2-pyridinamine;
4-[2-(4-Fluorophenyl)pyrazolo[1,5-a]pyridin-3-yl]-N,N-dimethyl-2-pyridinamine;
N-Benzyl-6-fluoro-4-[2-(4-fluorophenyl)pyrazolo[1,5-a]pyridin-3-yl]-2-pyridinamine;
4-[2-(4-Fluorophenyl)-6-trifluoromethylpyrazolo[1,5-a]pyridin-3-yl]-N-isopropyl-2-pyridinamine;
3-(4-[2-(4-Fluorophenyl)-6-trifluoromethylpyrazolo[1,5-a]-pyridin-3-yl]-2-pyridinylamino)-1-propanol;
N-(3-Aminopropyl)-4-[6-bromo-2-(4-fluorophenyl)-pyrazolo[1,5-a]pyridin-3-yl]-2-pyridinamine;
N-Butyl-4-[2-(4-fluorophenyl)pyrazolo[1,5-a]pyridin-3-yl]-2-pyrimidin-amine;
4-[2-(4-Fluorophenyl)pyrazolo[1,5-a]pyridin-3-yl]-N-(2-propyl)-2-pyrimidinamine;
4-[2-(4-Fluorophenyl)pyrazolo[1,5-a]pyridin-3-yl]-2-pyrimidinamine;
4-[2-(4-Fluorophenyl)-6-trifluoromethylpyrazolo[1,5-a]pyridin-3-yl]-2-pyrimidinamine;
N-Benzyl-4-[2-(4-fluorophenyl)-6-trifluoromethylpyrazolo[1,5-a]pyridin-3-yl]-2-pyrimidinamine;
N-Cyclopropyl-4-[2-(4-fluorophenyl)-6-trifluoromethylpyrazolo[1,5-a]pyridin-3-yl]-2-pyrimidinamine;
4-[2-(4-Fluorophenyl)-6-trifluoromethylpyrazolo[1,5-a]pyridin-3-yl]-N-(2,2,2-trifluoroethyl)-2-pyrimidinamine;
3-(4-[2-(4-Fluorophenyl)-6-trifluoromethylpyrazolo[1,5-a]pyridin-3-yl]-2-pyrimidinylamino)-1-propanol;
N-Cyclopropyl-4-[6-cyano-2-(4-fluorophenyl)pyrazolo[1,5-a]pyridin-3-yl]-2-pyrimidinamine;
2-(4-Fluorophenyl)-3-(4-(2-(3-hydroxypropyl)amino)pyrimidinyl)-6-pyrazolo-[1,5-a]pyridinylcarboxamide;
4-[2-(4-Fluorophenyl)pyrazolo[1,5-a]pyridin-3-yl]-N-[2-(1H-imidazol-5-yl)ethyl]-2-pyrimidinamine;
4-[2-(4-Fluorophenyl) pyrazolo[1,5-a]pyridin-3-yl]-N-(3-pyrid inyl-methyl)-2-pyrimidinamine;
4-[2-(4-Fluorophenyl)pyrazolo[1,5-a]pyridin-3-yl]-N-(2-pyridinylmethyl)-2-pyrimidinamine;
4-[2-(4-Fluorophenyl)pyrazolo[1,5-a]pyridin-3-yl]-N-(4-pyridinyl-methyl)-2-pyrimidinamine;
4-[2-(4-Fluorophenyl)pyrazolo[1,5-a]pyridin-3-yl]-N-pentyl-2-pyridinamine;
N-Butyl-4-[2-(4-fluorophenyl)-6-trifluoromethylpyrazolo-[1,5-a]pyridin-3-yl]-2-pyrimidinamine;
N-{4-[2-(4-fluorophenyl)-6-(trifluoromethyl)pyrazolo-[1,5-a]pyridin-3-yl]pyrimidin-2-yl}-N-[3-(4-methylpiperazin-1-yl)propyl]amine;
[3-[2-(Cyclopentylamino)-4-pyrimidinyl]-2-(4-fluorophenyl)pyrazolo[1,5-a]pyridin-6-yl]methanol;
N-Cyclopentyl-4-[2-(4-fluorophenyl)-6-methylpyrazolo[1,5-a]pyridin-3-yl]-2-pyrimidinamine;
N-Cyclopentyl-4-[6-[(cyclopentylamino)methyl]-2-(4-fluorophenyl) pyrazolo[1,5-a]pyridin-3-yl]-2-pyrimidinamine;
4-[5-Chloro-2-(4-fluorophenyl)pyrazolo[1,5-a]pyridin-3-yl]-N-cyclopentyl-2-pyrimidinamine;
N-Cyclopentyl-4-[2-(4-fluorophenyl)-5-(1-pyrrolidinyl)pyrazolo[1,5-a]pyridin-3-yl]-2-pyrimidinamine;
4-[5-Chloro-2-(4-methoxyphenyl)pyrazolo[1,5-a]pyridin-3-yl]-N-cyclopentyl-2-pyrimidinamine;
1-[3-({4-[2-(4-Fluorophenyl)-pyrazolo[1,5-a]pyridin-3-yl]-2-pyridinyl}amino)propyl]-2-pyrrolidinone;
6-Fluoro-4-[2-(4-fluorophenyl)-pyrazolo[1,5-a]pyridin-3-yl]-N-methyl-2-pyridinamine;
4-[4-Fluoro-2-(4-fluorophenyl)-pyrazolo[1,5-a]pyridin-3-yl]-N,N-dimethyl-2-pyridinamine;
N-Allyl-4-[2-(4-fluorophenyl)pyrazolo-[1,5-a]pyridin-3-yl]-2-pyridinamine;
5-[6-Chloro-2-(4-fluorophenyl)-pyrazolo[1,5-a]pyridin-3-yl]-N-cyclopropyl-2-pyridinamine;
3-({5-Bromo-4-[2-(4-fluorophenyl)-pyrazolo[1,5-a]pyridin-3-yl]-2-pyridinyl}amino)-1-propanol;
Methyl 3-(2-{[3-(acetyloxy)propyl]-amino}-4-pyridinyl)-2-(4-fluorophenyl)pyrazolo-[1,5-a]pyridine-6-carboxylate;
3-[2-(Cyclopropylamino)-4-pyrimidinyl]-2-(4-fluorophenyl)pyrazolo[1,5-a]pyridine-6-carboxylic acid;
3-[2-(Cyclopropylamino)-4-pyrimidinyl]-2-(4-fluorophenyl)-N,N-dimethylpyrazolo-[1,5-a]pyridine-6-carboxamide;
N-Cyclopropyl-3-[2-(cyclopropylamino)-4-pyrimidinyl]-2-(4-fluorophenyl)pyrazolo-[1,5-a]pyridine-6-carboxamide;
N-Cyclopentyl-4-[2-(4-fluorophenyl)pyrazolo[1,5-a]pyridin-3-yl]-6-phenyl-2-pyrimidinamine;
2-(4-Fluorophenyl)-3-(4-pyrimidinyl)-pyrazolo[1,5-a]pyridine;
2-(4-Fluorophenyl)-3-(4-(2-methylthio)pyrimidinyl)-pyrazolo[1,5-a]pyridine;
2-(4-Fluorophenyl)-3-(4-(2-methylsulfinyl)pyrimidinyl)-pyrazolo[1,5-a]pyridine;
2-(4-Fluorophenyl)-7-methyl-3-(4-pyrimidinyl)pyrazolo[1,5-a]pyridine;
2-(4-Fluorophenyl)-7-methylthio-3-(4-pyrimidinyl)pyrazolo[1,5-a]pyridine;
2-(4-Fluorophenyl)-7-methylsulfinyl-3-(4-pyrimidinyl)pyrazolo[1,5-a]-pyridine;
7-(2-Fluoroethoxy)-2-(4-fluorophenyl)-3-(4-(2-methylthio) pyrimidinyl)-pyrazolo[1,5-a]pyridine;
N-Butyl-4-[7-(2-fluoroethoxy)-2-(4-fluorophenyl)pyrazolo[1,5-a]pyridin-3-yl]-2-pyrimidinamine;
N-Benzyl-4-[7-(2-fluoroethoxy)-2-(4-fluorophenyl)pyrazolo[1,5-a]pyridin-3-yl]-2-pyrimidinamine;
2-(4-Fluorophenyl)-3-(4-(2-methylthio)pyrimidinyl)-7-(2,2,2-trifluoroethoxy)pyrazolo[
1,5-a]pyridine;
N-Butyl-4-[2-(4-fluorophenyl)-7-(2,2,2-trifluoroethoxy)pyrazolo[1,5-a]pyridin-3-yl]-2-pyrimidinamine;
N-Benzyl-4-[2-(4-fluorophenyl)-7-(2,2,2-trifluoroethoxy)pyrazolo[1,5-a]-pyridin-3-yl]-2-pyrimidinamine;
N-Cyclopropyl-4-[2-(4-fluorophenyl)-7-(2,2,2-trifluoroethoxy)pyrazolo[1,5-a]pyridin-3-yl]-2-pyrimidinamine;
N-Cyclopentyl-4-[2-(4-fluorophenyl)-7-(2,2,2-trifluoroethoxy)pyrazolo[1,5-a]pyridin-3-yl]-2-pyrimidinamine;
N-Cyclohexyl-4-[2-(4-fluorophenyl)-7-(2,2,2-trifluoroethoxy)pyrazolo[1,5-a]pyridin-3-yl]-2-pyrimidinamine;
3-(4-[2-(4-Fluorophenyl)-7-(2,2,2-trifluoroethoxy)pyrazolo[1,5-a]pyridin-3-yl]-2-pyrimidinylamino)-1-propanol;
2-(4-Fluorophenyl)-3-(4-(2-methyloxy)pyrimidinyl)-7-(2,2,2-trifluoroethoxy)pyrazolo[1,5-a]pyridine;
2-(4-Fluorophenyl)-3-(4-(2-phenyloxy)pyrimidinyl)-7-(2,2,2-trifluoroethoxy)pyrazolo[1,5-a]pyridine;
2-(4-Fluorophenyl)-3-(4-(2-(2,2,2-trifluoroethoxy))pyrimidinyl)-7-(2,2,2-trifluoroethoxy)pyrazolo[1,5-a]pyridine;
2-(4-Fluorophenyl)-3-(4-(2-methylsulfinyl)pyrimidinyl)-7-(ethylsulfinyl)-pyrazolo[1,5-a]pyridine;
2-(4-Fluorophenyl)-3-(4-(2-methylthio)pyrimidinyl)-7-(ethylthio)-pyrazolo[1,5-a]pyridine;
7-(2-Fluoroethoxy)-2-(4-fluorophenyl)-3-(4-(2-methylsulfinyl)-pyrimidinyl)-pyrazolo[1,5-a]pyridine;
2-(4-Fluorophenyl)-3-(4-(2-methylsulfinyl)pyrimidinyl)-7-(2,2,2-trifluoroethoxy)-pyrazolo[1,5-a]pyridine;
2-(4-Fluorophenyl)-3-(4-pyridyl)-pyrazolo[1,5-a]pyridine;
2-(4-Fluorophenyl)-7-methyl-3-(4-pyridinyl)pyrazolo[1,5-a]pyridine;
2-(4-Fluorophenyl)-7-methoxy-3-(4-pyridinyl)pyrazolo[1,5-a]-pyridine;
2-(4-Fluorophenyl)-3-(2-fluoro-4-pyridinyl)-7-methoxypyrazolo[1,5-a]pyridine;
N-Butyl-4-[2-(4-fluorophenyl)-7-methoxypyrazolo[1,5-a]pyridin-3-yl]-2-pyridinamine;
N-{4-[5-Chloro-7-(ethylsulfanyl)-2-(4-fluorophenyl)pyrazolo[1,5-a]pyridin-3-yl]-2-pyrimidinyl}-N-cyclopentylamine;
Ethyl 3-[2-(cyclopentylamino)-4-pyrimidinyl]-2-(4-fluorophenyl)-7-methylpyrazolo[1,5-a]pyridine-6-carboxylate;
3-[2-(Cyclopentylamino)-4-pyrimidinyl]-2-(4-fluorophenyl)-7-methylpyrazolo[1,5-a]pyridine-6-carboxylic acid;
3-[2-(Cyclopentylamino)-4-pyrimidinyl]-N-cyclopropyl-2-(4-fluorophenyl)-7-methylpyrazolo[1,5-a]pyridine-6-carboxamide;
N-Butyl-4-[7-butyl-2-(4-fluorophenyl)pyrazolo[1,5-a]pyridin-3-yl]-2-pyrimidinamine;
N-Butyl-4-[2-(4-fluorophenyl)-7-methylpyrazolo[1,5-a]pyridin-3-yl]-2-pyrimidinamine;
N-Butyl-4-[2-(4-fluorophenyl)-7-octylpyrazolo[1,5-a]pyridin-3-yl]-2-pyrimidinamine;
N-Cyclopropyl-4-[7-ethyl-2-(4-fluorophenyl)pyrazolo[1,5-a]pyridin-3-yl]-2-pyrimidinamine;
Dimethyl 2-(4-fluorophenyl)-3-(4-(2-cyclopropylamino)pyrimidinyl)-7-pyrazolo[1,5-a]pyridinylcarboxamide;
N-Cyclopentyl-4-[2-(4-fluorophenyl)-5-morpholin-4-ylpyrazolo[1,5-a]pyridin-3-yl]pyrimidin-2-amine;
N¹-{4-[2-(4-Fluorophenyl)-6-trifluoromethylpyrazolo[1,5-a]pyridin-3-yl]pyrimidin-2-yl}-N³,N³-dimethylpropane-1,3-diamine;
3-(2-Butoxypyridin-4-yl)-2-(4-fluorophenyl)pyrazolo[1,5-a]pyridine;
N-Cyclopentyl-4-[2-(2,4-dimethoxyphenyl)pyrazolo[1,5-a]pyridin-3-yl]pyrimidin-2-amine;
5-Bromo-4-[5-chloro-2-(4-fluorophenyl)pyrazolo[1,5-a]pyridin-3-yl]-N-cyclopentylpyrimidin-2-amine;
N-Cyclopentyl-6-[2-(4-fluorophenyl)pyrazolo[1,5-a]pyridin-3-yl]pyrimidin-4-amine;
N-Cyclopropyl-4-[2-(4-methoxyphenyl)-6-(trifluoromethyl) pyrazolo[1,5-a]pyridin-3-yl]pyrimidin-2-amine;
N-Cyclopropyl-4-[2-(methoxyphenyl)-6-(triethoxymethyl)pyrazolo[1,5-a]pyridin-3-yl]pyrimidin-2-amine;
Ethyl 3-[2-(cyclopropylamino)pyrimidin-4-yl]-2-(4-methoxyphenyl)-pyrazolo[1,5-a]pyridine-6-carboxylate;
3-[2-(Cyclopropylamino)pyrimidin-4-yl]-N-(2-methoxyethyl)-2-(4-methoxyphenyl)pyrazolo[1,5-a]pyridine-6-carboxamide;
4-{5-Chloro-2-[4-(cyclopropyl methoxy)phenyl]pyrazolo[1,5-a]pyridin-3-yl}-N-cyclopropyl-2-pyrimidinamine;
4-[7-Butoxy-2-(4-methoxyphenyl)pyrazolo[1,5-a]pyridin-3-yl]-N-cyclopentyl-2-pyrimidinamine;
4-[5-Chloro-2-(3-chlorophenyl)-7-(methylsulfanyl)pyrazolo[1,5-a]pyridin-3-yl]-N-cyclopentyl-2-pyrimidinamine; and
N-cyclopentyl-6-[2-(4-fluorophenyl)-7-(methylthio)pyrazolo[1,5-a]pyridin-3-yl]pyrimidin-4-amine.

It will be appreciated by those skilled in the art that the compounds of the present invention may also be utilized in the form of a pharmaceutically acceptable salt, solvate or phsiologically functional derivative thereof. The pharmaceutically acceptable salts of the compounds of formula (I) include conventional salts formed from pharmaceutically acceptable inorganic or organic acids or bases as well as quaternary ammonium salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts.

The present invention provides compounds of formula (I) for use in medical therapy, e.g. in the treatment or prophylaxis, including suppression of recurrence of symptoms, of a viral disease in an animal, e.g. a mammal such as a human. The compounds of formula (I) are especially useful for the treatment or prophylaxis of viral diseases such as herpes viral infections. Herpes viral infections include, for example, herpes simplex virus 1 (HSV-1), herpes simplex virus 2 (HSV-2), cytomegalovirus (CMV), Epstein Barr virus (EBV), varicella zoster virus (VZV), human herpes virus 6 (HHV-6), human herpes virus 7 (HHV-7), and human herpes virus 8 (HHV-8). The compounds of the invention are useful in the treatment or prophylaxis of the symptoms or effects of herpes virus infections.

The compounds of the invention are useful in the treatment or prophylaxis of conditions or diseases associated with herpes virus infections, particularly conditions or diseases associated with latent herpes virus infection in an animal, e.g., a mammal such as a human. By conditions or diseases associated with herpes viral infections is meant a condition or disease, excluding the viral infection per se, which results from the presence of the viral infection, such as chronic fatigue syndrome, which is associated with EBV infection, multiple sclerosis (MS) which has been associated with herpes viral infections such as EBV and HHV-6. Other examples of conditions or diseases that are associated with herpes virus infection include those described in the background section above.

In addition to those conditions and diseases, the compounds of the present invention may also be used for the treatment or prophylaxis of cardiovascular diseases and conditions associated with herpes virus infections, in particular atherosclerosis, coronary artery disease and restenosis and specifically restenosis following angioplasty (RFA). Restenosis is the narrowing of the blood vessels which can occur after injury to the vessel wall, for example injury caused by balloon angioplasty or other surgical and/or diagnostic techniques, and is characterized by excessive proliferation of smooth muscle cells in the walls of the blood vessel treated. It is thought that in many patients suffering from RFA, viral infection, particularly by CMV and/or HHV-6 of the patient plays a pivotal role in the proliferation of the smooth muscle cells in the coronary vessel treated. Restenosis can occur following a number of surgical and/or diagnostic techniques, for example, transplant surgery, vein grafting, coronary by-pass grafting and, most commonly following angioplasty.

There is evidence from work done both in vitro and in vivo, indicating that restenosis is a multifactorial process. Several cytokines and growth factors, acting in concert, stimulate the migration and proliferation of vascular smooth muscle cells (SMC) and production of extracellular matrix material, which accumulate to occlude the blood vessel. In addition growth suppressors act to inhibit the proliferation of SMC's and production of extracellular matrix material.

In addition, compounds of formula (I) may be useful in the treatment or prophylaxis of Hepatitis B and Hepatitis C viruses, human pappiloma virus (HPV) and human immunodeficiency virus (HIV).

The present invention provides a method for the treatment or prophylaxis of a viral infection in an animal such as a mammal (e.g., a human), particularly a herpes viral infection, which comprises administering to the animal a therapeutically effective amount of the compound of formula (I).

As used herein, the term “prophylaxis” refers to the complete prevention of infection, the prevention of occurrence of symptoms in an infected subject, the prevention of recurrence of symptoms in an infected subject, or a decrease in severity or frequency of outward symptoms of viral infection or disease in the subject.

As used herein, the term “treatment” refers to the partial or total elimination of symptoms or decrease in severity of symptoms of viral infection, condition or disease in the subject, or the elimination or decrease of viral presence in the subject.

As used herein, the term “therapeutically effective amount” means an amount of a compound of formula (I) which is sufficient, in the subject to which it is administered, to treat or prevent the stated disease, condition or infection. For example, a therapeutically effective amount of a compound of formula (I) for the treatment of a herpes virus infection is an amount sufficient to treat or prevent the herpes virus infection in the subject.

The present invention also provides a method for the treatment or prophylaxis of conditions or diseases associated with herpes viral infections in an animal such as a mammal (e.g., a human), which comprises administering to the animal a therapeutically effective amount of the compound of formula (I). In one embodiment, the present invention provides a method for the treatment or prophylaxis of chronic fatigue syndrome and multiple sclerosis in an animal such as a mammal (e.g., a human), which comprises administering to the animal a therapeutically effective amount of a compound of formula (I). The foregoing method is particularly useful for the treatment or prophylaxis of chronic fatigue syndrome and multiple sclerosis associated with latent infection with a herpes virus.

In another embodiment, the present invention provides a method for the treatment or prophylaxis of a cardiovascular condition such as atherosclerosis, coronary artery disease or restenosis (particularly restenosis following surgery such as angioplasty), which comprises administering to the animal a therapeutically effective antiviral amount of the compound of formula (I).

The present invention further provides a method for the treatment or prophylaxis of hepatitis B or hepatitis C viruses in an animal such as a mammal (e.g., a human), which comprises administering to the animal a therapeutically effective amount of the compound of formula (I).

The present invention further provides a method for the treatment or prophylaxis of human papilloma virus in an animal such as a mammal (e.g., a human), which comprises administering to the animal a therapeutically effective amount of the compound of formula (I).

The present invention further provides a method for the treatment or prophylaxis of HIV in an animal such as a mammal (e.g., a human), which comprises administering to the animal a therapeutically effective amount of the compound of formula (I).

The present invention also provides the use of the compound of formula (I) in the preparation of a medicament for the treatment or prophylaxis of a viral infection in an animal such as a mammal (e.g., a human), particularly a herpes viral infection; the use of the compound of formula (I) in the preparation of a medicament for the treatment of conditions or diseases associated with a herpes viral infection; and the use of the compound of formula (I) in the preparation of a medicament for the treatment or prophylaxis of hepatitis B or hepatitis C viruses, human papilloma virus and HIV. In particular, the present invention also provides the use of a compound of formula (I) in the preparation of a medicament for the treatment or prophylaxis of chronic fatigue syndrome or multiple sclerosis. In one embodiment, the present invention provides the use of a compound of formula (I) in the preparation of a medicament for the treatment or prophylaxis of cardiovascular disease, such as restenosis and atherosclerosis.

The compounds of formula (I) are conveniently administered in the form of pharmaceutical compositions. Such compositions may conveniently be presented for use in any conventional manner in admixture with one or more physiologically acceptable carriers or diluents.

While it is possible that compounds of the present invention may be therapeutically administered as the raw chemical, it is preferable to present the active ingredient as a pharmaceutical formulation or composition for convenient administration. Such compositions may conveniently be presented for use in conventional manner in admixture with one or more pharmaceutically acceptable carriers or diluents. The carrier(s) must be “acceptable” in the sense of being compatible with the other ingredients of the formulation and not deleterious to the recipient thereof.

Accordingly, the present invention further provides for a pharmaceutical composition or formulation comprising a compound of formula (I) with one or more pharmaceutically acceptable carriers or diluents therefore and, optionally, other therapeutic and/or prophylactic ingredients.

The formulations include those suitable for oral, parenteral (including subcutaneous e.g. by injection or by depot tablet, intradermal, intrathecal, intramuscular e.g. by depot and intravenous), rectal and topical (including dermal, buccal and sublingual) administration although the most suitable route may depend upon for example the condition, age, and disorder of the recipient as well as the viral infection or disease being treated. The formulations may conveniently be presented in unit dosage form and may be prepared by any of the methods well known in the art of pharmacy. All methods include the step of bringing into association the compound(s) (“active ingredient”) with the carrier or diluent and optionally one or more accessory ingredients. In general the formulations are prepared by uniformly and intimately bringing into association the active ingredient with liquid carriers or diluents or finely divided solid carriers or diluents or both and then, if necessary, shaping the product into the desired formulation.

Formulations suitable for oral administration may be presented as discrete units such as capsules, cachets or tablets (e.g. chewable tablets in particular for paediatric administration) each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste. A tablet may be made by compression or moulding, optionally with one or more accessory ingredients. Compressed tablets may be prepared by compressing in a suitable machine the active ingredient in a free-flowing form such as a powder or granules, optionally mixed with other conventional excipients such as binding agents, (for example, syrup, acacia, gelatin, sorbitol, tragacanth, mucilage of starch or polyvinylpyrrolidone), fillers (for example, lactose, sugar, microcrystalline cellulose, maize-starch, calcium phosphate or sorbitol), lubricants (for example, magnesium stearate, stearic acid, talc, polyethylene glycol or silica), disintegrants (for example, potato starch or sodium starch glycollate) or wetting agents, such as sodium lauryl sulfate. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The tablets may be coated according to methods well-known in the art.

Alternatively, the compounds of the present invention may be incorporated into oral liquid preparations such as aqueous or oily suspensions, solutions, emulsions, syrups or elixirs, for example. Moreover, formulations containing these compounds may be presented as a dry product for constitution with water or other suitable vehicle before use. Such liquid preparations may contain conventional additives such as suspending agents such as sorbitol syrup, methyl cellulose, glucose/sugar syrup, gelatin, hydroxyethylcellulose, carboxymethyl cellulose, aluminum stearate gel or hydrogenated edible fats; emulsifying agents such as lecithin, sorbitan mono-oleate or acacia; non-aqueous vehicles (which may include edible oils) such as almond oil, fractionated coconut oil, oily esters, propylene glycol or ethyl alcohol; and preservatives such as methyl or propyl p-hydroxybenzoates or sorbic acid. Such preparations may also be formulated as suppositories, e.g., containing conventional suppository bases such as cocoa butter or other glycerides.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations may be presented in unit-dose or multi-dose containers, for example sealed ampoules and vials, and may be stored in a freeze-dried (lyophilised) condition requiring only the addition of a sterile liquid carrier, for example, water-for-injection, immediately prior to use. Extemporaneous injection solutions and suspensions may be prepared from sterile powders, granules and tablets of the kind previously described. Formulations for rectal administration may be presented as a suppository with the usual carriers such as cocoa butter, hard fat or polyethylene glycol. Formulations for topical administration in the mouth, for example buccally or sublingually, include lozenges comprising the active ingredient in a flavoured base such as sucrose and acacia or tragacanth, and pastilles comprising the active ingredient in a base such as gelatin and glycerin or sucrose and acacia.

The compounds may also be formulated as depot preparations. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

In addition to the ingredients particularly mentioned above, the formulations may include other agents conventional in the art having regard to the type of formulation in question, for example those suitable for oral administration may include flavouring agents.

It will be appreciated that the amount of a compound of the invention required for use in treatment will vary with the nature of the condition being treated and the age and the condition of the patient and will be ultimately at the discretion of the attendant physician or veterinarian. In general, however, doses employed for adult human treatment will typically be in the range of 0.02-5000 mg per day, preferably 100-1500 mg per day. The desired dose may conveniently be presented in a single dose or as divided doses administered at appropriate intervals, for example as two, three, four or more sub-doses per day. The formulations according to the invention may contain between 0.1-99% of the active ingredient, conveniently from 30-95% for tablets and capsules and 3-50% for liquid preparations.

The compound of formula (I) for use in the instant invention may be used in combination with other therapeutic agents for example, non-nucleotide reverse transcriptase inhibitors, nucleoside reverse transcriptase inhibitors, protease inhibitors and/or other antiviral agents. The invention thus provides in a further aspect the use of a combination comprising a compound of formula (I) with a further therapeutic agent in the treatment of viral infections. Particular antiviral agents which may be combined with the compounds of the present invention include aciclovir, valaciclovir, famcyclovir, gancyclovir, docosanol, miribavir, amprenavir, lamivudine, zidovudine, and abacavir. Preferred antiviral agents for combining with the compounds of the present invention include aciclovir and valaciclovir. Thus the present invention provides in a further aspect, a combination comprising a compound of formula (I) and an antiviral agent selected from the group consisting of aciclovir or valaciclovir; the use of such combination in the treatment of viral infections and the preparation of a medicament for the treatment of viral infections, and a method of treating viral infections comprising administering a compound of formula (I) and an antiviral agent selected from the group consisting of aciclovir and valaciclovir.

When the compound of formula (I) is used in combination with other therapeutic agents, the compounds may be administered either sequentially or simultaneously by any convenient route.

The combinations referred to above may conveniently be presented for use in the form of a pharmaceutical formulation and thus pharmaceutical formulations comprising a combination as defined above optionally together with a pharmaceutically acceptable carrier or diluent comprise a further aspect of the invention. The individual components of such combinations may be administered either sequentially or simultaneously in separate or combined pharmaceutical formulations.

When combined in the same formulation it will be appreciated that the two compounds must be stable and compatible with each other and the other components of the formulation and may be formulated for administration. When formulated separately they may be provided in any convenient formulation, in such a manner as are known for such compounds in the art.

When a compound of formula (I) is used in combination with a second therapeutic agent active against the viral infection, the dose of each compound may differ from that when the compound is used alone. Appropriate doses will be readily appreciated by those skilled in the art.

The compounds employed in this present invention may be made by a variety of methods, utilizing standard chemistry. Any previously defined variable will continue to have the previously defined meaning unless otherwise indicated. Illustrative general synthetic methods are set out below and then specific compounds of the invention are prepared in the working Examples.

For example, a general method (A) for preparing the compounds of Formula (I) comprises the reaction of a compound of Formula (VII) 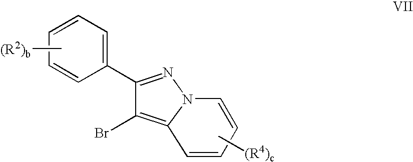
with a compound of Formula (VIII) or a compound of Formula (IX): 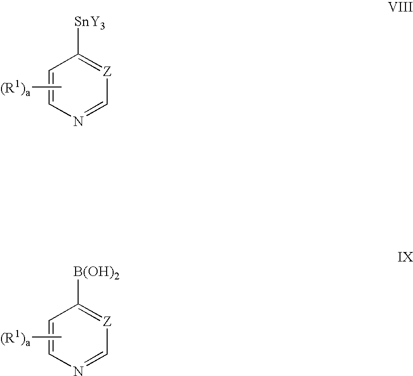
wherein Z is CH or N and Y is methyl or butyl.

This general method (A) can be conveniently performed by mixing the two compounds in an inert solvent, in the presence of a palladium catalyst, and optionally heating the mixture to about 100° C. Preferably the reaction is performed using an approximately equimolar mixture of (VII) and (VIII), or an approximately equimolar mixture of (VII) and (IX). The palladium catalyst is preferably present in the proportion of 1-5 mol % compared to (VII). Palladium catalysts which may be used include, but are not limited to, tetrakistriphenylphosphine palladium(0), bis(triphenylphosphine)palladium dichloride. When one of the reactant partners is a compound of formula (IX), the reaction is more conveniently carried out by adding a base in a proportion equivalent to, or greater than, that of (IX). Preferably the base is a trialkylamine or sodium hydrogen carbonate.

Another general method (B) for the preparation of the compounds of this invention is the reaction of a compound of Formula (VII) with a compound of Formula (X) as summarized below to give compounds of Formula (I) where R¹ is hydrogen.

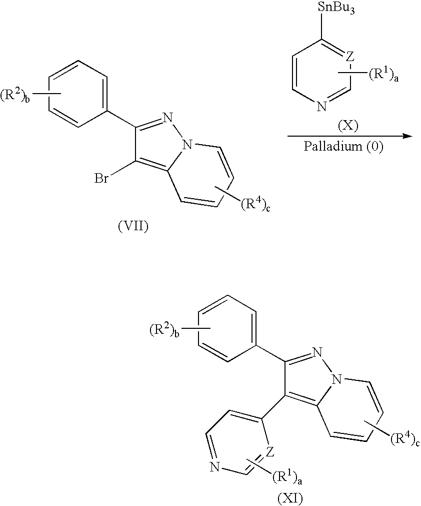

The type of reaction utilized in general method (B) is well documented in the literature and is routinely referred to as a ‘Stille’ coupling (Stille, Angew. Chem. Int. Ed. Engl. 1986, 25, 508). This reaction is brought about by mixing the two reactants in an inert solvent in the presence of a catalytic quantity of a palladium species and heating the reaction mixture. Conveniently the solvent is, for example, toluene, dioxane, tetrahydrofuran or dimethylformamide and the palladium catalyst is a palladium(0) species, or a convenient precursor thereof, for example, tetrakis(triphenylphosphine)palladium(0) or bis(triphenylphosphine)palladium dichloride. For example, when R⁴ is hydrogen, the reaction is most conveniently performed by mixing the two reactants, in an approximate equimolar ratio, in toluene, adding an amount of tetrakis(triphenylphosphine)palladium(0) equal to about 5 mol % of that of (VII), and heating the mixture at about 100-120° C. until the reaction is judged complete by the disappearance of either (VII) or (X). Typically this reaction requires between 12 and 48 hours to proceed to completion. The product can be conveniently isolated using procedures typical for this Stille coupling procedure.

One skilled in the art will recognize that a similar reaction, illustrated below in general method (C) can be used to prepare compounds of the invention using boron containing reactants such as (XII).

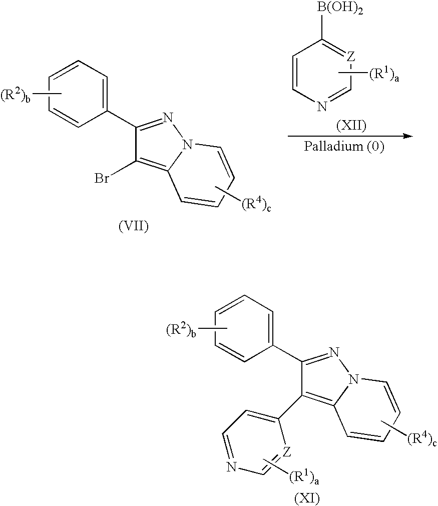

The use of boronic acids, or esters, in such a coupling reaction is typically referred to as a ‘Suzuki’ coupling reaction (Suzuki, A. et al. Synth. Commun. 1981, 11, 513). Said reaction is conveniently brought about by mixing the two reactants, in an inert solvent, in the presence of a catalytic quantity of a palladium species and a base, and heating the reaction mixture. Conveniently the solvent is, for example, toluene, dioxane, tetrahydrofuran or dimethylformamide and the palladium catalyst is a palladium(0) species, or a convenient precursor thereof, for example, tetrakis(triphenylphosphine)palladium(0) or bis(triphenylphosphine)palladium dichloride, and the base is sodium bicarbonate or a trialkyl amine such as triethyl amine.

Boron containing compounds such as (XII) and tin containing compounds such as (X) are either commercially available or can be prepared using methods known to one skilled in the art (Stille, Angew. Chem. Int. Ed. Engl. 1986, 25, 508; Snieckus, V. et al. J. Org. Chem. 1995, 60, 292-6).

Compounds of formula (VII) may be conveniently prepared from compounds of Formula (XII) by a decarboxylation/bromination sequence as shown below.

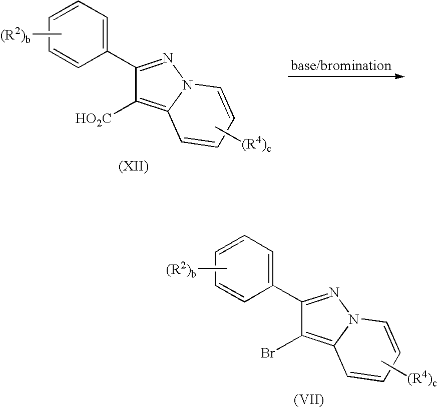

This reaction can be achieved by treatment of a compound of formula (XII), dissolved in a suitable solvent, with a base followed by a brominating agent and stirring the mixture at, or about, 25° C. until the reaction is judged complete by the disappearance of (XII). Suitable solvents include, but are not limited to, dimethylformamide, dimethylacetamide, dioxane and the like. Conveniently the base is sodium hydrogen carbonate and the brominating agent can be, for example, N-bromosuccinimide. Alternatively, compounds of formula (VII) can be conveniently prepared by treatment of a compound of formula (XIII) with a brominating agent as summarized below.

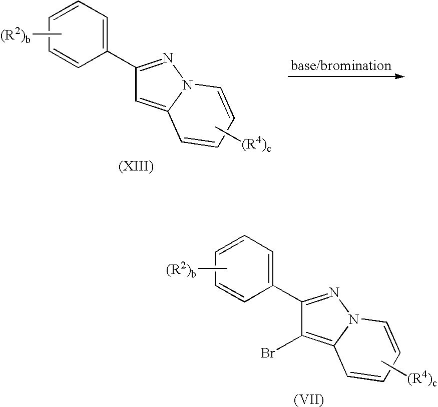

This reaction can be easily carried out by dissolving the compound of formula (XIII) in an inert solvent and adding to the solution a brominating agent in sufficient quantity to effect complete reaction of (XIII). Preferably the solvent is dimethylformamide, dimethylacetamide, dioxane and the like and brominating agents include, but are not limited to, bromine, N-bromosuccinimide, N-bromoacetamide and the like.

Compounds of formula (XIII) may be conveniently prepared by the decarboxylation of a compound of formula (XII) as summarized below.

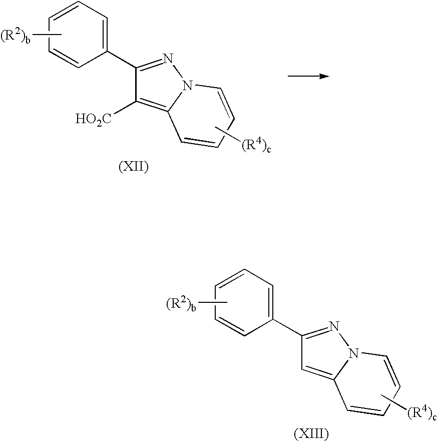

Said decarboxylation may be carried out by any one of a variety of methods described in the literature for similar decarboxylations. For example: heating a solution of a compound of formula (XII) in an inert solvent, or conversion to a ‘Barton ester’ followed by treatment with a radical reductant, for example tributyltin hydride (Crich, D. Aldrichimica Acta, 1987, 20, 35).

Compounds of formula (XII) can be prepared most readily by simple hydrolysis of lower alkyl esters of formula (XIV). Esters such as (XIV) are commonly referred to as pyrazolo[1,5-a]pyridines (Hardy, C. R. Adv. Het. Chem. 1984, 36, 343) and may be prepared by a cycloaddition reaction between compounds of formula (XV) and acetylenes of formula (XVI), as summarized below.

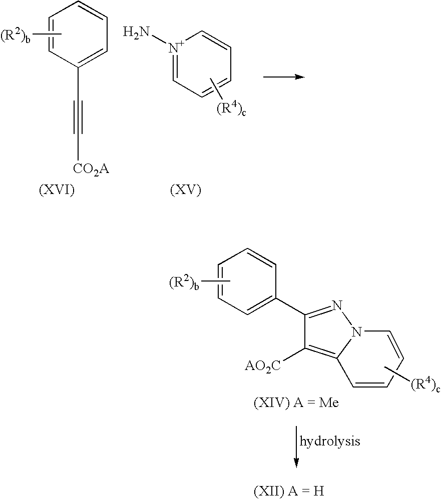

Cycloaddition reactions such as these are commonly known as [3+2] dipolar cycloaddition reactions. Conveniently the reaction may be carried out by mixing the reactants (XV) and (XVI), in equimolar amounts, in an inert solvent and adding a suitable base. The mixture is then stirred at between 20-100° C. until the reaction is judged complete by the disappearance of one of the reactants. Preferred solvents include but are not limited to acetonitrile, dioxane, tetrahydrofuran, dimethylformamide and the like. Preferred bases include non-nucleophilic amines such as 1,8-diazabicyclo[5.4.0]undec-7-ene, 1,5-diazabicyclo[4.3.0]non-5-ene, 1,4-diazabicyclo[2.2.2]octane and the like.

Esters such as those of Formula (XIV) can be conveniently hydrolyzed to their corresponding carboxylic acids by standard hydrolysis conditions employed to effect similar hydrolysis reactions (Larock, Comprehensive Organic Transformations, 1989, 981). For example, treatment of a solution of a compound of formula (XIV) in a lower alcohol, for example methanol, with sodium hydroxide followed by heating the mixture for an appropriate time gives the compound of formula (XII).

Compounds of formula (XV) are aminated pyridine derivatives and are either commercially available or can be conveniently prepared by reacting a suitable pyridine with an aminating reagent such as O-(mesitylsulfonyl)hydroxylamine, O-(diphenylphosphinyl)hydroxylamine and the like.

Acetylenic esters such as those of formula (XVI) are either known compounds or can be prepared by methods described in the literature. Preferred methods include the reaction of acetylenes such as those of formula (XVII) with a suitable base to generate an acetylenic anion and subsequent reaction of the anion with an alkoxycarbonylating agent, as summarized below.

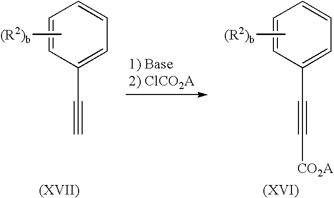

Preferably the acetylene (XVII) is dissolved in an inert solvent, such as tetrahydrofuran, and the solution is cooled to about −75° C. A base is added in sufficient quantity to effect deprotonation of the acetylene (XVII). The preferred bases include, but are not limited to, n-butyllithium, lithium diisopropylamide, sodium bis(trimethylsilyl)amide and the like. To the reaction mixture is then added a reagent capable of reacting with an anion to introduce an alkoxycarbonyl group. Preferred reagents include, but are not limited to, methyl chloroformate, ethyl chloroformate, benzyl chloroformate and the like. Arylalkynes such as (XVII) are either known compounds or can be prepared by literature methods such as those described in, for example, Negishi, E. J. Org. Chem. 1997, 62, 8957.

Compounds of formula (XIII) can also be prepared via a number of other convenient routes. Disubstituted acetylenes as represented by formula (XVIII) can be treated with an aminating agent, optionally in the presence of a base, to give compounds of formula (XIII). The aminating agent is, preferably, O-(mesitylsulfonyl)hydroxylamine and the base is potassium carbonate.

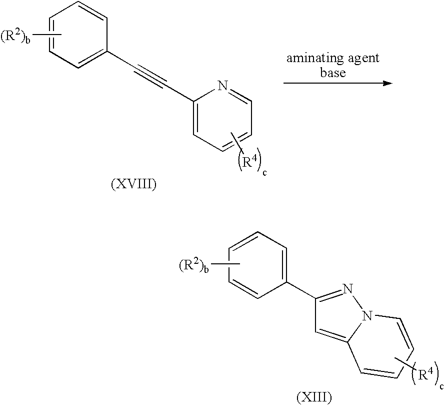

Disubstituted acetylenes such as (XVIII) are readily prepared by a palladium catalyzed coupling reaction between aryl acetylenes and 2-halopyridines using methods described in the literature (Yamanake et al, Chem. Pharm. Bull. 1988, 1890).

An alternative synthesis of compounds of formula (XIII) involves treating a ketone of formula (XIX) with an aminating agent in a suitable solvent and optionally heating the reaction. The aminating agent is, preferably, O-(mesitylsulfonyl)hydroxylamine and preferred solvents include chloroform, dichloromethane and the like.

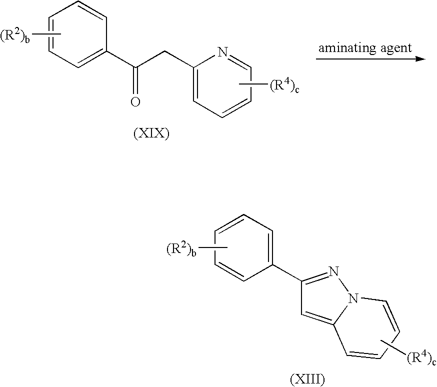

Ketones such as those of formula (XIX) can be readily prepared using procedures described in the literature (Cassity, R. P.; Taylor, L. T.; Wolfe, J. F. J. Org. Chem. 1978, 2286).

A more preferred approach to compounds of formula (XIII) involves the conversion of ketones of formula (XIX) to oximes such as (XX) followed by treatment of said oximes with an aminating agent

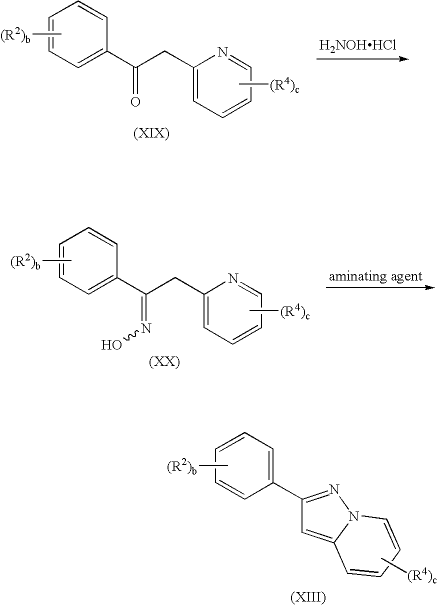

Typically, oximes of formula (XX) are readily prepared by treating ketones of formula (XIX) with a source of hydroxylamine, in an appropriate solvent, and optionally in the presence of a base. Preferably the source of hydroxylamine is hydroxylamine hydrochloride and the base is sodium carbonate, potassium carbonate, or an aqueous solution of sodium hydroxide. Preferred solvents include lower alcohols, such as methanol and ethanol, or acetonitrile. The aminating agent is, preferably, O-(mesitylsulfonyl)hydroxylamine and preferred solvents include chloroform, dichloromethane and the like.

A still more preferred method for the preparation of compounds of formula (XIII) from oximes of formula (XX) involves the treatment of the said oximes with an acylating or sulfonylating agent in the presence of a base to generate azirines of formula (XXI).

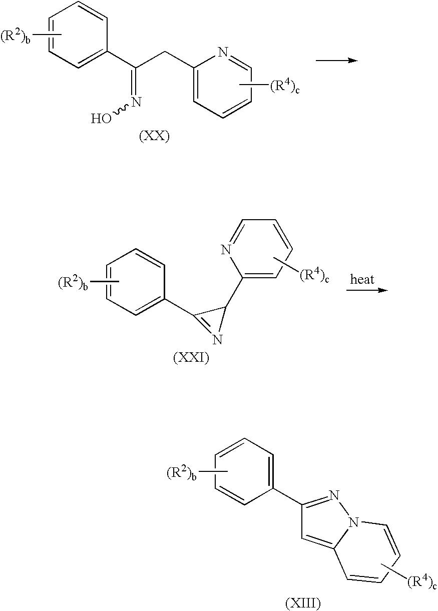

Azirines such as (XXI) can be rearranged to compounds of formula (XIII) by heating a solution of said azirine in a suitable solvent at temperatures of about 100-180° C. More preferably the rearrangement is carried out in the presence of FeCl₂. In the presence of FeCl₂ the rearrangement occurs at lower temperatures and in a higher yield. Typically the azirines (XXI) can be prepared by treatment of oximes of formula (XX) with acetic anhydride, trifluoroacetic anhydride, methanesulfonyl chloride, toluenesulfonyl chloride and the like in an inert solvent, for example, chloroform, dichloromethane or toluene. Preferred bases include, but are not limited to, triethylamine, diisopropylethylamine, pyridine and the like.

A general method (D) for the preparation of compounds of formula (V) comprises the reaction of a compound of formula (XXII) with a compound of formula (XXIII). 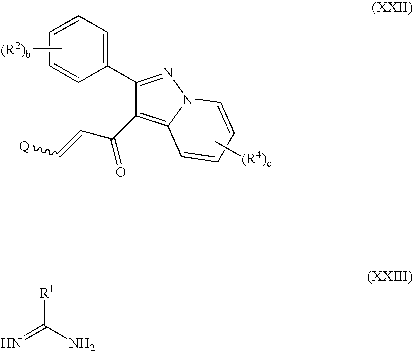
wherein Q is alkyloxy, alkylthio or dialkylamino.

The general method (D) can be readily carried out by mixing a compound of formula (XXII) with a compound of formula (XXIII) in a suitable solvent, optionally in the presence of a base, and heating the reaction mixture to about 50-150° C. Typically the solvent is a lower alcohol such as methanol, ethanol, isopropanol and the like, and the base can be, for example, a sodium alkoxide, potassium carbonate or an amine base such as triethylamine.

Compounds of formula (XXII) may be conveniently prepared by reacting a compound of formula (XXIV) with a dimethylformamide dialkylacetal, to give compounds of formula (XXII) wherein Q is Me₂N, or with a trialkyl orthoformate or a dialkoxymethyl acetate, to give compounds of formula (XXII) wherein Q is an alkoxy group. Conveniently, a dimethylformamide dialkylacetal is dimethylformamide dimethyl acetal or dimethylformamide di-tert-butyl acetal and the reaction carried out by mixing the compound of formula (XXIV) with the dimethylformamide dialkylacetal and optionally heating the reaction. Preferred trialkyl orthoformates include trimethyl orthoformate and triethyl orthoformate. In a similar manner, diethoxymethyl acetate can be employed to prepare compounds of formula (XXII) wherein Q is EtO—.

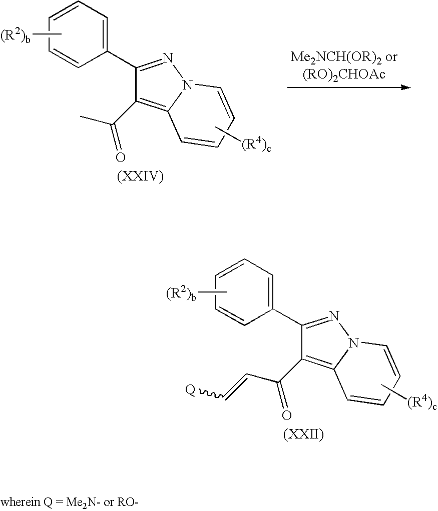

Compounds of formula (XXIV) can be prepared from compounds of formula (XIII) by an acylation procedure.

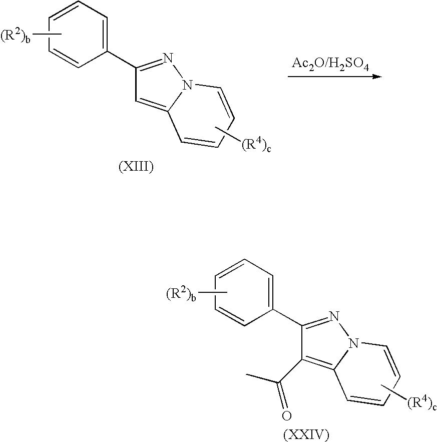

Typically the acylation is conveniently carried out by treating the compounds of formula (XIII) with an acylating agent optionally in the presence of an acid catalyst. The preferred acylating agent is acetic anhydride (“Ac₂O”) and a convenient acid is sulfuric acid.

Methods for the synthesis of compounds of formula (XIII) are described above.

Certain compounds of formula (V) may be conveniently prepared by a process which involves reacting a ketone of formula (XXV) with an N-aminopyridine derivative in the presence of an acid or a base. Typically the acid is p-toluenesulfonic acid and the base can be potassium carbonate, sodium hydroxide, cesium carbonate, lithium hydroxide, triethylamine, potassium tert-butoxide.

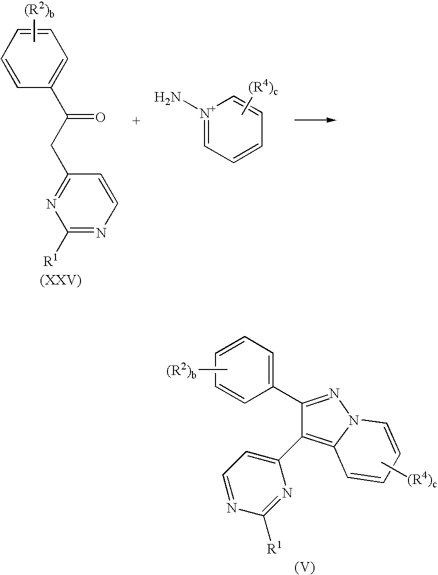

Compounds of formula (I) can also be converted to other compounds of formula (I). For example, reaction of a compound of formula (XXXVII) with a non-nucleophilic base, for example n-butyllithium, followed by treatment with an electrophilic agent gives compounds of formula (XXXVIII), as summarized below.

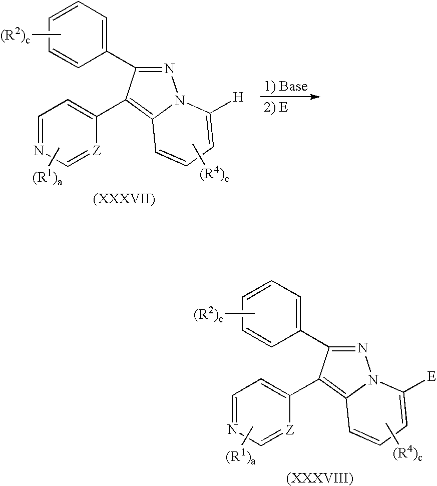

Electrophiles which can be successfully used in this procedure include, but are not limited to: alkyl halides (E=methyl, benzyl etc.); N-bromosuccinimide (E=bromine); N-chlorosuccinimide (E=chlorine); N-iodosuccinimide (E=iodine); aldehydes (E=CH(OH)R); dimethylformamide (E=CHO); dimethyl disulfide (E=SMe); carbon dioxide (E=CO₂H); dimethylcarbamoyl chloride (E=C(O)NMe₂) and the like.

Compounds of formula (XXXVIII), wherein E is a halogen such as chloride (XXXVIII-A), or a sulfone, such as p-tolylsulfonyl, can be transformed into compounds in which E is an ether group by treatment of said chloro, or p-tolylsulfonyl, derivative with alcohols as summarized below.

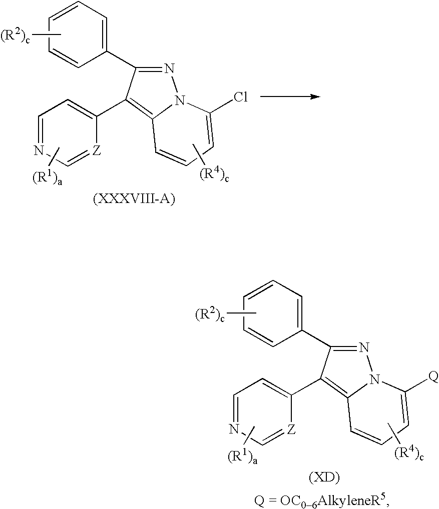

This transformation is most conveniently carried out by mixing the chloride of formula (XXXVIII-A) with an excess of the alcohol, optionally in the presence of an inert solvent, and heating the mixture to about 100-150° C.

As another example, compounds of formula (I) wherein R¹ is a leaving group, for example a halogen such as chloride, or a sulfone such as methanesulfonyl can be converted into compounds of formula (I) wherein R¹ is an ether or an amino group by treatment of said chloro, or methanesulfonyl derivative with alcohols or amines. Thus, a particularly preferred method for synthesising compounds of formula (V) wherein R¹ is —NH—(CH₂)ₑ—R⁵ is shown below.

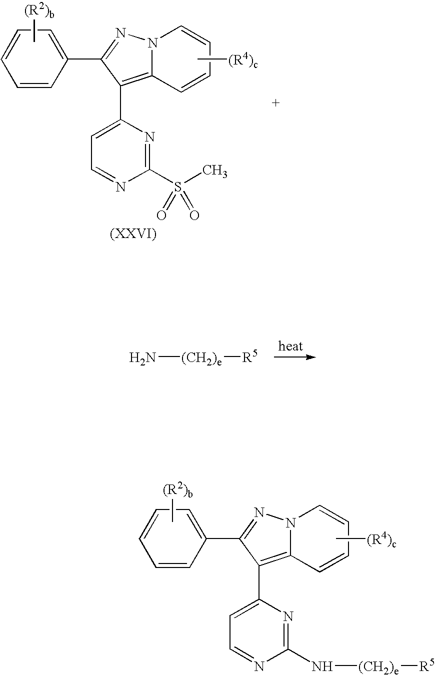

A compound of formula (XXVI) is mixed at room temperature with a neat amine of general formula H₂N—(CH₂)ₑ—R₅. The mixture is then heated with an airgun until a homogenoeous melt is obtained. This usually takes about 2 minutes. Upon cooling, water is added and the compound of formula (I) precipates out and may be separated by filtration.

Compounds of formula (XXVI) may be produced by the reaction of oxone with compounds of formula (XXVII) as shown below.

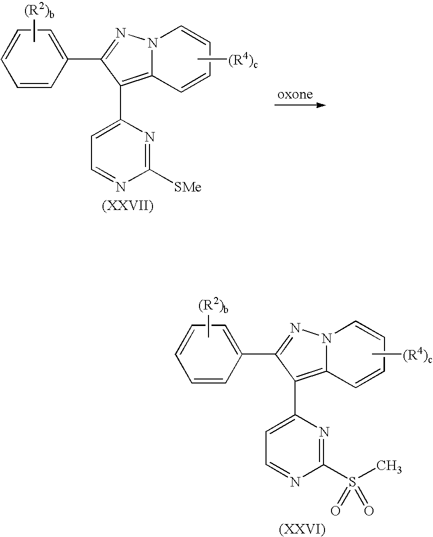

Compounds of formula (XXVII) may be produced by reaction of a compound of formula (VII) with a compound of formula (VIII) wherein Z is N, R¹ is —SMe and Y is butyl. The synthesis of a compound of formula (VIII) wherein Z is N, R¹ is —SMe and Y is butyl is described in the literature (Sandosham, J. and Undheim, K. Tetrahedron 1994, 50, 275; Majeed, A J. et al Tetrahedron 1989, 45, 993).

Compounds of formula (I), wherein R⁴ is hydrogen can be converted into compounds wherein R⁴ is bromide or iodide and is attached to position 6. Said conversion is conveniently carried out by addition of a brominating agent such as N-bromosuccinimide, or an iodinating agent such as N-iodosuccinimide, to a solution of a compound of formula (XXX) in an appropriate solvent. Preferred solvents include dimethylformamide, dichloromethane and the like.

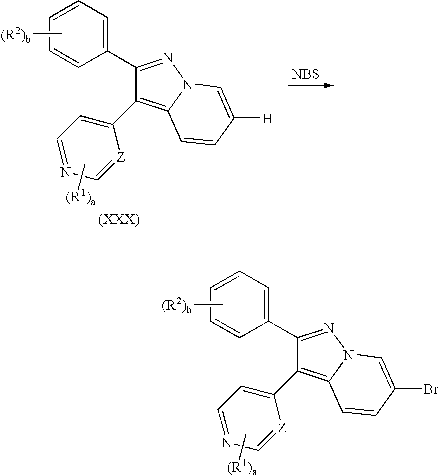

Compounds of formula (I), wherein R⁴ is a bromide or iodide and is attached to position 6 or 7 can be converted to compounds with different substitutions at position 6 or 7, respectively, by a variety of methods. For example, treatment of a compound of formula (XXXI), wherein R⁴ is bromide or iodide, under conditions well known in the art as Stille coupling reactions or Suzuki coupling reactions leads to compounds wherein R⁴ is alkyl, alkenyl, alkynyl, cyano, or carboalkoxy.

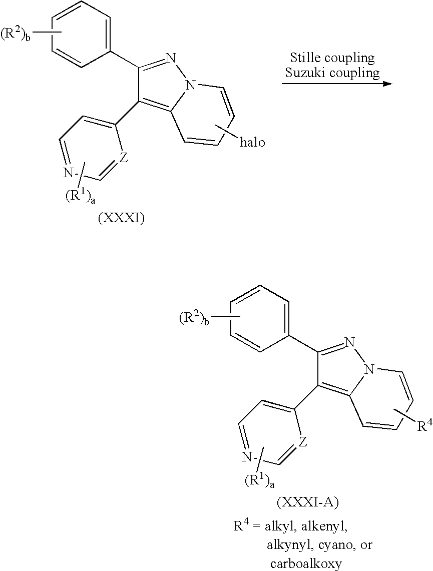

Compounds of formula (XXXI-A) wherein R⁴ is a trifluoromethyl group (CF₃) can be converted into compounds wherein R⁴ is a carboxylic acid derivative. Preferably said transformation is carried out by treatment of a compound of formula (XXXII) with a suitable base in an alcoholic solvent and optionally heating the reaction to about 80° C. Preferably the base is a sodium or potassium alkoxide such as sodium ethoxide and the like and the preferred solvents include, but are not limited to, methanol, ethanol, propanol, isopropanol and the like. The resulting trialkylorthoesters can be converted to lower alkyl esters by treatment of said orthoesters in a suitable solvent with an acid in the presence of water. Preferred acids include p-toluenesulfonic acid, hydrochloric acid and sulfuric acid and the preferred solvents include lower alcohols and acetone. Lower alkyl esters such as those represented by formula (XXXIII) can be further converted into different compounds by transformation of the ester group in a manner well known in the art.

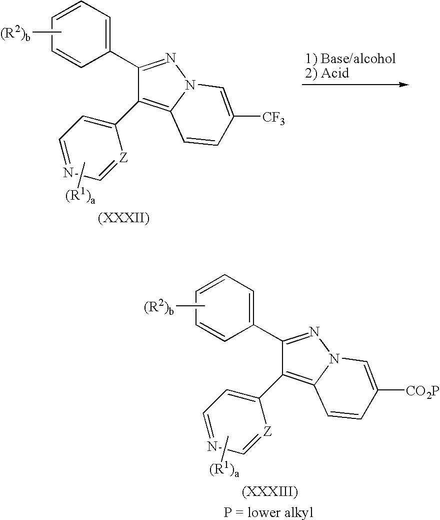

Compounds of formula (I), wherein R¹, R² or R⁴ contains a hydroxyl group can be reacted to give compounds wherein the hydroxyl group is converted to an ester, carbonate or carbamate group using procedures well known in the literature (March J. Advanced Organic Chemistry).

Similarly, compounds of general formula (I), wherein R¹, R² or R⁴ contains an amino group attached to the pyrazolopyridine ring through another functional group, can be reacted to give compounds wherein the amino group is converted to an amide, carbamate or urea group using procedures known in the literature (March J. Advanced Organic Chemistry).

Certain compounds of formula (I) wherein at least one R² group is substituted on an ortho position of the phenyl ring may be prepared by the reaction of a compound of formula (XXXIV) wherein Y is methyl or butyl: 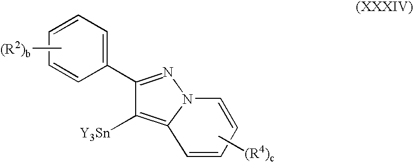
with a compound of formula (XXXV):

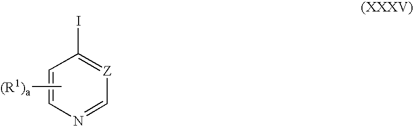

This reaction is essentially the reverse of the coupling reaction described above between compounds of formula (VIII) and (IX). The reaction conditions are analogous to those previously described for the coupling reaction between compounds of formula (VIII) and (IX).

Compound (XXXIV) wherein Y is butyl may be prepared from a compound of formula (VII) using a strong base, butyl lithium and tri-n-butyl stannyl chloride at low temperature (e.g. −78° C.) in an inert solvent such as tetrahydrofuran (THF).

The following examples are intended for illustration only and are not intended to limit the scope of the invention in any way. Reagents are commercially available or are prepared according to procedures in the literature. Example numbers refer to those compounds listed in the tables above. ¹H and ¹³C NMR spectra were obtained on Varian Unity Plus NMR spectrophotometers at 300 or 400 MHz, and 75 or 100 MHz respectively. ¹⁹F NMR were recorded at 282 MHz. Mass spectra were obtained on Micromass Platform, or ZMD mass spectrometers from Micromass Ltd. Altrincham, UK, using either Atmospheric Chemical Ionization (APCI) or Electrospray Ionization (ESI). Analytical thin layer chromatography was used to verify the purity of some intermediates which could not be isolated or which were too unstable for full characterization, and to follow the progress of reactions. Unless otherwise stated, this was done using silica gel (Merck Silica Gel 60 F254). Unless otherwise stated, column chromatography for the purification of some compounds, used Merck Silica gel 60 (230-400 mesh), and the stated solvent system under pressure. All compounds were characterized as their free-base form unless otherwise stated. On occasion the corresponding hydrochloride salts were formed to generate solids where noted.

### EXAMPLE 1

### 4-[2-(4-Fluorophenyl)pyrazolo[1,5-a]pyridin-3-yl]-N-[2-(1H-imidazol-5-yl)ethyl]-2-pyridinamine

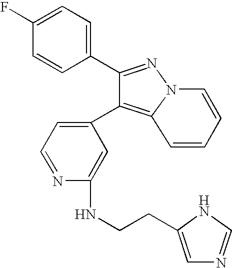

### a) 1-(4-Fluorophenyl)-2-trimethylsilylacetylene

4-Fluoroiodobenzene (112 mL, 0.97 mol) and triethylamine (176 mL, 1.26 mol) are dissolved in dry THF (1.2 L) and nitrogen gas was bubbled through the solution for about 20 min. Copper (I) iodide (1.08 g, 5.7 mmol) and bis(triphenyphosphine)-palladium dichloride (2.15 g, 3 mmol) are added and then trimethylsilylacetylene (178 mL, 1.3 mol) was added dropwise over about 40 min with the temperature being maintained at about 23° C. A large amount of precipitate forms (presumably Et₃NHCl) which necessitates mechanical stirring. Following complete addition of the trimethylsilylacetylene the mixture was allowed to stir at room temperature for about 18 hours. The mixture was filtered and the solid washed with cyclohexane. The combined filtrates are concentrated under reduce pressure to give a brown oil. Application of this oil to a pad of silica gel followed by elution with cyclohexane gave a yellow solution. Removal of the solvent gave the title compound as a yellow oil; 182.8 g (95%).

### b) Methyl 3-(4-fluorophenyl)propiolate

A solution of 1-(4-fluorophenyl)-2-trimethylsilylacetylene (64 g, 0.33 mol) in dry diethyl ether (400 mL) was cooled to 0° C. under a nitrogen atmosphere. To this solution was added, dropwise over 45 minutes, a solution of tetrabutylammonium fluoride (1M in THF, 330 mL, 0.33 mol) via a dropping funnel maintaining the internal temperature below 2° C. The mixture was allowed to warm to room temperature over about 1 hour. Diethyl ether (300 mL) was added to the mixture and the organic solution was washed with water, saturated brine and then dried over anhydrous magnesium sulfate. The magnesium sulfate was removed by filtration and the filtrate was cooled to about −78° C. n-Butyl lithium (1.6M in hexanes, 450 mL, 0.72 mol) was added dropwise via a dropping funnel over about 1 hour while the temperature was maintained below −66° C. After complete addition the mixture was stirred at −78° C. for about 1 hour and then a precooled solution of methyl chloroformate (110 mL, 1.4 mol) in dry diethyl ether (200 mL) was added in a continuous stream as fast as possible. The mixture was allowed to cool to −78° C. and then allowed to warm to room temperature over 1.5 hours. The organic reaction mixture was washed with water and saturated brine and then dried over anhydrous magnesium sulfate. The solvents are remove under reduced pressure and the residue dried under reduced pressure to give the title compound as a brown solid, 36.5 g (61%). ¹H NMR (CDCl3) δ 7.58 (dd, 2H, J=9, 5.4 Hz), 7.07 (t, 2H, J=8.5 Hz), 3.84 (s, 3H). MS (+ve ion electrospray) 178 (30), (M⁺).

### c) Methyl 2-(4-fluorophenyl)-pyrazolo[1,5-a]pyridine-3-carboxylate

A stirred solution of methyl 3-(4-fluorophenyl)propiolate (8.02 g, 45 mmol) and 1-aminopyridinium iodide (10 g, 45 mmol) in dry acetonitrile (150 mL) was cooled to about 0° C. A solution of 1,8-diazabicycloundec-7-ene (13.7 g, 90 mmol) in dry acetonitrile (50 mL) was added dropwise over 1 hour. The mixture was allowed to stir at room temperature for about 18 hours. The reaction mixture was cooled in an ice bath for about 30 min. and the precipitate was collected by filtration and washed with cold acetonitrile (10 mL). The solid was dried under reduced pressure to give the title compound as a white solid, 8.48 g (70%). ¹H NMR (CDCl3) δ 8.50 (d, 1H, J=8.4 Hz), 8.18 (d, 1H, J=8.8 Hz), 7.78 (m, 2H), 7.42 (t, 1H, J=8.4 Hz), 7.13 (t, 2H, J=8.8 Hz), 6.97 (td, 1H, J=6.8, 1 Hz). MS (+ve ion electrospray) 271 (100), (MH⁺).

### d) 2-(4-Fluorophenyl)-pyrazolo[1,5-a]pyridine-3-carboxylic Acid

A solution of methyl 2-(4-fluorophenyl)-pyrazolo[1,5-a]pyridine-3-carboxylate (5.0 g, 18.5 mmol) in 2N aqueous sodium hydroxide (50 ml) and methanol (30 mL) was heated at reflux for about 3 hours. The mixture was filtered and the filtrate was washed with diethyl ether (20 mL) and then concentrated under reduced pressure to about half the original volume. Concentrated hydrochloric acid was added to adjust the pH to about 2 and the resulting solid was collected by filtration and washed with water and dried under vacuum to give the title compound as a white solid, 4.8 g (ca. 100%). ¹H NMR (d6 DMSO) δ 12.43 (s, 1h), 8.84 (d, 1H, J=6.9 Hz), 8.14 (d, 1H, J=9 Hz), 7.82 (m, 2H), 7.57 (t, 1H, J=8.1 Hz), 7.28 (t, 2H, J=9 Hz), 7.15 (td, 1H, J=6.9, 1.2 Hz). MS (+ve ion electrospray) 257 (100), (MH⁺).

### e) 2-(4-Fluorophenyl)-3-bromopyrazolo[1,5-a]pyridine

To a solution of 2-(4-fluorophenyl)-pyrazolo[1,5-a]pyridine-3-carboxylic acid (0.96 g, 3.75 mmol) in dry DMF (10 mL) was added sodium bicarbonate (0.95 g, 11.3 mmol) followed by N-bromosuccinimide (0.667 g, 3.75 mmol) and the mixture was stirred at room temperature under a nitrogen atmosphere for about 90 min. The mixture was poured into water (300 mL) and the resulting solid was collected by filtration and washed with water. The solid was dissolved in 10:1 chloroform:methanol (10 mL) and filtered through a pad (0.5 cm) of silica gel using 10:1 chloroform:methanol as eluent. The filtrate was evaporated to leave the title compound as a tan solid, 0.87 g (80%). ¹H NMR (d6 DMSO) δ 8.7 (d, 1H, J=6.9 Hz), 8.02 (dd, 2H, J=8.7, 5.7 Hz), 7.61 (d, 1H, J=8.4 Hz), 7.40 (t, 1H, J=6 Hz), 7.38 (t, 2H, J=9 Hz), 7.04 (t, 1H, J=6.9 Hz). MS (+ve ion electrospray) 293 (100), (MH⁺).

### f) 2-Fluoropyridin-4-ylboronic Acid

To a stirred solution of n-butyl lithium (3.2 mL, 2.5M, 8.0 mmol) in dry diethyl ether (20 mL) at −78° C. was added a solution of 2-fluoro-4-iodopyridine (1.5 g, 6.7 mmol) in dry ether (10 mL) and the reaction mixture was stirred at −78° C. for 10 min. Tributyl borate (2.4 mL, 2.01 g, 8.7 mmol) was added and the reaction mixture was allowed to stir to room temperature over 2 hours. Water (5 mL) was added followed by 2N aqueous sodium hydroxide solution (10 mL) to dissolve the solids. The organic phase was separated. The aqueous phase was acidified to pH 3 using 6N HCl and the resulting white solid was collected by filtration and dried under vacuum to give the title compound, 0.74 g (78%). ¹H NMR (DMSO-d6) δ 8.65 (s, 2H), 8.21 (d, 1H, J=4.8 Hz), 7.59 (t, 1H, J=4.8 Hz), 7.37 (d, 1H, J=1.8 Hz).

### g) 2-(4-Fluorophenyl)-3-(2-fluoro-4-pyridinyl)pyrazolo[1,5-a]-pyridine

A solution of 3-bromo-2-(4-fluorophenyl)-pyrazolo[1,5-a]pyridine (1.30 g, 4.5 mmol), 2-fluoro-4-pyridinylboronic acid (694 mg, 4.9 mmol) and dichlorobis(triphenylphosphine)-palladium (316 mg, 0.45 mmol) in dimethylformamide (DMF) (100 mL) was placed in a pre-heated oil bath at 110° C. To the reaction was added, in a dropwise manner, 2M aqueous sodium carbonate (4.5 mL, 9.0 mmol). The reaction was allowed to stir for 2 hours and then cooled to room temperature and filtered through a pad of Celite. The Celite pad was washed with ethyl acetate and the filtrate was concentrated to dryness at 50° C. under vacuum. The residue was partitioned between ethyl acetate and water. The layers were separated and the organic phase was dried over anhydrous magnesium sulfate. The drying agent was removed by filtration and the filtrate was concentrated and purified by silica gel chromatography to yield the title compound (378 mg, 1.23 mmol, 27%). ¹H NMR (CDCl3): δ 8.57(d, 1H, J=6.9 Hz), 8.22(d, 1H, J=5.4 Hz), 7.7(d, 1H, J=9.0 Hz), 7.75(m, 2H), 7.33(m, 1H), 7.14(m, 3H), 6.95(m, 2H). MS (ES+ve) 308 (100, M⁺).

h) In a sealed-tube was combined 2-(4-fluorophenyl)-3-(2-fluoro-4-pyridinyl)-pyrazolo[1,5-a]pyridine (30 mg, 0.10 mmol) and histamine (40 mg, 0.36 mmol), and the reaction was placed in a pre-heated oil bath at 140° C. The reaction was stirred at 140° C. until consumption of starting material was indicated by TLC analysis (50% ethyl acetate in hexanes). The contents of the sealed-tube were transferred to a flask and concentrated to dryness at 50° C. under high vacuum. The residue was purified by silica gel chromatography to yield the title compound, 23 mg (0.06 mmol, 60%). ¹H NMR (d₆-dmso): δ 11.8 (s, 1H), 8.73 (d, 1H, J=6.8 Hz), 7.94 (d, 1H, J=5.3 Hz), 7.63 (d, 1H, J=9.3 Hz), 7.57 (dd, 2H, J=5.3, 8.6 Hz), 7.48 (s, 1H), 7.30 (t, 1H, J=7.6 Hz), 7.23 (t, 2H, J=9.0 Hz), 6.97 (t, 1H, J=6.8 Hz), 6.75 (s, 1H), 6.57 (m, 1H, J=5.3 Hz), 6.44 (s, 1H), 6.33 (d, 1H, J=5.3 Hz), 3.41 (q, 2H, J=6.6 Hz), 2.7 (t, 2H, J=6.6 Hz). MS (ES+ve): 399.1 (50, M+), 305.3 (90), 169.4 (100).

### EXAMPLE 2

### N-Butyl-4-[2-(4-fluorophenyl)pyrazolo[1,5-a]pyridin-3-yl]-2-pyridinamine

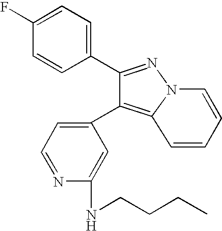

In a similar manner as described in Example 1(h), using butylamine in place of histamine, was obtained the title compound. ¹H NMR (CD₂Cl₂): δ 8.49 (d, 1H. J=7.2 Hz), 8.01 (d, 1H, J=5.2 Hz), 7.62 (m, 3H), 7.21(m, 1H), 7.07(t, 2H, J=8.8 Hz), 6.85 (m, 2H), 6.54 (dd, 1H. J=4.8, 0.8 Hz), 6.32 (s, 1H), 3.16 (quart, 2H, J=6.4 Hz), 1.53 (quint, 2H, J=7.2 Hz), 1.37 (sext, 2H, J=Hz), 0.92 (t, 3H, J=7.2 Hz). MS (ES+ve) 361 (100, M⁺).

### EXAMPLE 3

### 3-(4-[2-(4-Fluorophenyl)pyrazolo[1,5-a]pyridin-3-yl]-2-pyridinylamino)-1-propanol

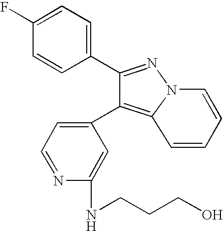

In a similar manner as described in Example 1(h), using 3-hydroxypropylamine in place of histamine, was obtained the title compound. ¹H NMR (CD₂Cl₂): δ 8.55 (d, 1H, J=6.9 Hz), 8.04 (d, 1H, J=5.4 Hz), 7.66 (m, 3H), 7.26 (m, 2H), 7.13 (t, 2H, J=8.7 Hz), 6.90 (t, 1H, J=6.9 Hz), 6.57 (d, 1H, J=5.1 Hz), 6.43 (s, 1H), 4.50 (t, 1H, J=5.7 Hz), 3.66 (t, 2H, J=5.7 Hz), 3.55 (quart, 2H, J=6.0 Hz), 1.76 (quint, 2H, J=5.7 Hz). MS (ES+ve): 363 (100, M⁺).

### EXAMPLE 4

### N¹-4-[2-(4-Fluorophenyl)pyrazolo[1,5-a]pyridin-3-yl]-2-pyridinyl-1,3-propanediamine

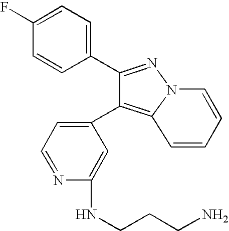

In a similar manner as described in Example 1(h), using 1,3-diaminopropane in place of histamine, was obtained the title compound. ¹H NMR (CD₂Cl₂): δ 8.55(d, 1H, J=5.4 Hz), 8.08(d, 1H, J=3.9 Hz), 7.69(m, 3H), 7.25 (dd, 1H, J=5.7, 8.7), 7.12(t, 2H, J=6.6 Hz), 6.9(t, 1H, J=6.9 Hz), 6.59(d, 1H, J=5.7 Hz), 6.4(s, 1H), 5.02(m, 1H), 3.33(q, 2H, J=5.1 Hz), 2.82(t, 2H, J=5.4 Hz), 1.72(m, 2H, J=5.4 Hz). MS (ES+ve): 362 (100, M⁺).

### EXAMPLE 5

### 4-[2-(4-Fluorophenyl)pyrazolo[1,5-a]pyridin-3-yl]-N-hexyl-2-pyridinamine

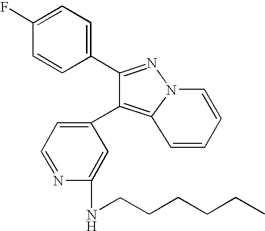

In a similar manner as described in Example 1(h), using hexylamine in place of histamine, was obtained the title compound. ¹H NMR (acetone-d₆): δ 8.67 (d, 1H, J=7.2 Hz), 8.05 (d, 1H, J=5.4 Hz), 7.72 (m, 3H), 7.33 (dd, 1H, J=7.2, 8.4 Hz), 7.21 (t, 2H, J=9.0 Hz), 7.00 (td, 1H, J=6.9, 0.9 Hz), 6.50 (s, 1H), 6.49 (d, 1H, J=5.1 Hz), 5.85 (t, 1H, J=5.1 Hz), 3.34 (quart, 2H, J=6.0 Hz), 1.61 (quint, 2H, J=6.9 Hz), 1.36 (m, 6H), 0.92 (t, 3H, J=2.4 Hz). MS (ES+ve): 389 (100, M⁺).

### EXAMPLE 6

### 4-[2-(4-Fluorophenyl)pyrazolo[1,5-a]pyridin-3-yl]-N-(4-methoxybenzyl)-2-pyridinamine

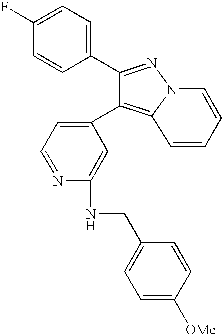

In a similar manner as described in Example 1(h), using 4-methoxybenzylamine in place of histamine, was obtained the title compound. ¹H NMR (Dimethyl-d₆ sulfoxide): δ 8.79 (d, 1H, J=7.2 Hz), 7.98 (d, 1H, J=5.4 Hz), 7.62 (dd, 2H, J=5.4, 8.4 Hz), 7.53 (d, 1H, J=9.0 Hz), 7.29 (m, 5H), 7.04 (quart, 2H, J=5.7 Hz), 6.92 (d, 2H, J=8.7 Hz), 6.51 (s, 1H), 6.38 (d, 1H, J=5.1 Hz).

### EXAMPLE 7

### 4-[2-(4-Fluorophenyl)pyrazolo[1,5-a]pyridin-3-yl]-N-(3-pyridinylmethyl)-2-pyridinamine

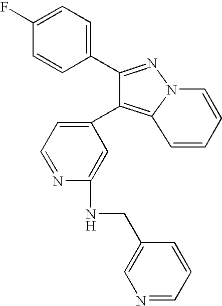

In a similar manner as described in Example 1(h), using 3-(aminomethyl)-pyridine in place of histamine, was obtained the title compound. ¹H NMR (acetone-d₆): δ 8.50 (d, H. J=6.8 Hz), 8.32 (d, H, J=4.0 Hz), 7.90 (d, H, J=5.2 Hz), 7.63 (d, H, J=7.6 Hz), 7.52 (m, H), 7.46 (d, H, J=9.2 Hz), 7.16 (m, H), 7.04 (t, H, J=8.8 Hz), 6.85 (t, H, J=6.4 Hz), 6.45 (s, H), 6.37 (d, H, J=4.4 Hz). MS (ES+ve): 396 (60, M⁺), 109 (100).

### EXAMPLE 8

### 4-[2-(4-Fluorophenyl)pyrazolo[1,5-a]pyridin-3-yl]-N-propyl-2-pyridinamine

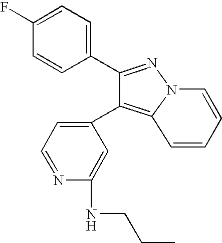

In a similar manner as described in Example 1(h), using propylamine in place of histamine, was obtained the title compound. ¹H NMR (acetone-d₆): δ 8.67 (d, 1H, J=7.2 Hz), 8.05 (d, 1H, J=5.1 Hz), 7.72 (m, 3H), 7.35 (dd, 1H, J=6.9, 9.0 Hz), 7.22 (t, 2H, J=9.0 Hz), 7.03 (t, 1H, J=6.6 Hz), 6.51 (s, 1H), 6.50 (d, H, J=7.2 Hz), 5.84 (m, 1H), 3.31 (quart, 2H, J=6.6 Hz), 1.63 (sext, 2H, J=7.2 Hz), 0.98 (t, 3H, J=Hz). MS (ES+ve): 347 (100, M⁺).

### EXAMPLE 9

### 2-(4-[2-(4-Fluorophenyl)pyrazolo[1,5-a]pyridin-3-yl]-2-pyridinylamino)-1-ethanol

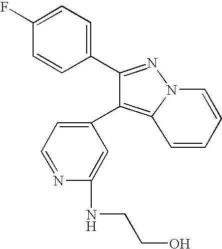

In a similar manner as described in Example 1(h), using 2-hydroxyethylamine in place of histamine, was obtained the title compound. ¹H NMR (DMSO-d6): δ 8.79 (d, 1H, J=6.9 Hz), 7.96 (d, 1H, J=5.4 Hz), 7.69 (d, 1H, J=9.0 Hz), 7.62 (m, 2H), 7.36 (dd, 1H, J=8.7, 6.9 Hz), 7.29 (m, 2H), 7.03 (t, 1H, J=6.6 Hz), 6.56 (m, 2H), 6.36 (d, 1H. J=5.1 Hz), 3.53 (t, 2H, J=5.7 Hz), 3.34 (m, 2H). MS (ES+ve): 349 (100, M⁺). MS (ES+ve): 437 (100, M⁺).

### EXAMPLE 10

### N-Benzyl-4-[2-(4-fluorophenyl)pyrazolo[1,5-a]pyridin-3-yl]-2-pyridinamine

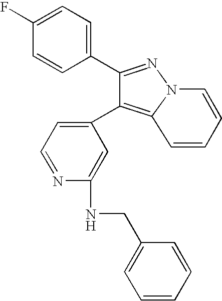

In a similar manner as described in Example 1(h), using benzylamine in place of histamine, was obtained the title compound. ¹H NMR (acetone-d₆): δ 8.65 (d, 1H, J=6.9 Hz), 8.06 (d, 1H, J=5.1 Hz), 7.70 (m, 2H), 7.54 (d, 1H, J=8.7 Hz), 7.31 (m, 7H), 7.01 (t, 1H, J=6.9 Hz), 6.58 (s, 1H), 6.51 (dd, 1H, J=1.5, 5.1 Hz), 6.38 (m, 1H), 4.62 (m, 2H). MS (ES+ve): 395 (100, M⁺).

### EXAMPLE 11

### 4-[2-(4-Fluorophenyl)pyrazolo[1,5-a]pyridin-3-yl]-N,N-dimethyl-2-pyridinamine

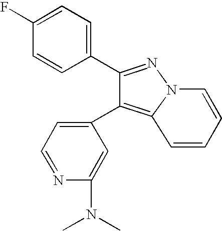

In a similar manner as described in Example 1(h), using N,N-dimethylamine in place of histamine, was obtained the title compound. ¹H NMR (CD₂Cl₂): δ 8.55(d, 1H, J=9.3 Hz), 8.17(d, 1H, J=6.5 Hz), 7.64-7.74(m, 3H), 7.25(dd, 1H, J=8, 11.5 Hz), 7.12(t, 2H, J=11.5 Hz), 6.90(t, 1H, J=9.3 Hz), 6.57(d, 1H, J=6.5 Hz), 6.54(s, 1H), 3.06(s, 6H). MS (ES+ve): 333.2 (100, M⁺).

### EXAMPLE 12

### N-Benzyl-6-fluoro-4-[2-(4-fluorophenyl)pyrazolo[1,5-a]pyridin-3-yl]-2-pyridinamine

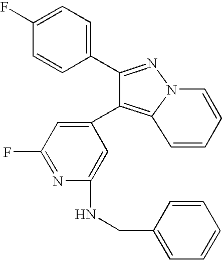

### a) 3-(2,6-Difluoro-4-pyridinyl)-2-(4-fluorophenyl)pyrazolo[1,5-a]pyridine

A solution of 3-bromo-2-(4-fluorophenyl)-pyrazolo[1,5-a]pyridine (from Example 1(e), 570 mg, 1.96 mmol), 2,6-difluoro-4-pyridyl-boronic acid (340 mg, 2.15 mmol) and dichlorobis(triphenylphosphine)palladium (137 mg, 0.196 mmol) in DMF (10.0 mL) was placed in a pre-heated oil bath at 110° C. To the reaction was added, in a dropwise manner, 2M sodium carbonate (2.00 mL, 4.00 mmol). The reaction was allowed to stir for 45 minutes before cooling to room temperature and filtering through a Celite 545 pad. The Celite filter was washed with ethyl acetate and the filtrate was concentrated to dryness at 50° C. under vacuum. The residue was dissolved in methylene chloride and dried over anhydrous magnesium sulfate. The drying agent was removed by filtration and the filtrate was concentrated and purified by silica gel chromatography to yield the title compound (160 mg, 0.492 mmol, 25%). ¹H NMR (CDCl₃): δ 8.53(d, 1H, J=6.8 Hz), 7.67(d, 1H, J=8.8 Hz), 7.53(dd, 2H, J=5.6, 8.0 Hz), 7.31 (t, 1H, J=7.6 Hz), 7.11 (t, 2H, J=8.4 Hz), 6.93(t, 1H, J=6.8 Hz), 6.75(s, 2H). MS (ES+ve): 326 (90, M⁺).

b) In a sealed-tube was combined 3-(2,6-difluoro-4-pyridinyl)-2-(4-fluorophenyl)pyrazolo[1,5-a]pyridine (35 mg, 0.11 mmol) and benzylamine (3.0 mL, 2.9 g, 27 mmol), and the reaction was placed in a pre-heated oil bath at 130° C. The reaction was stirred at 130° C. until consumption of starting material was indicated by TLC analysis (50% ethyl acetate in hexanes). The contents of the sealed-tube was transferred to a flask and concentrated to dryness at 50° C. under high vacuum. The residue was purified by silica gel chromatography to yield the title compound, 18 mg (0.04 mmol, 36%). ¹H NMR (d₆-acetone): δ 8.67(d, 1H, J=6.8 Hz), 7.71(dd, 2H, J=5.6, 8.8 Hz), 7.59(d, 1H, J=8.8 Hz), 7.30-7.45(m, 6H), 7.24(t, 2H, J=8.8 Hz), 7.05(t, 1H, J=6.8 Hz), 6.73(m, 1H, J=6.0 Hz), 6.46(s, 1H), 6.09(s, 1H), 4.59(d, 2H, J=6.0 Hz). MS (ES+ve): 413.1 (100, M⁺).

### EXAMPLE 13

### 4-[2-(4-Fluorophenyl)-6-trifluoromethylpyrazolo[1,5-a]pyridin-3-yl]-N-isopropyl-2-pyridinamine

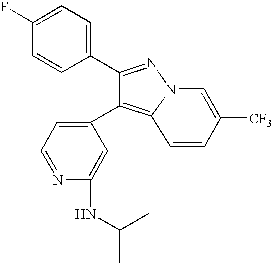

### a) 2-(4-Fluorophenyl)-3-(2-fluoro-4-pyridinyl)-6-trifluoromethylpyrazolo[1,5-a]pyridine

In a similar manner as described in Example 1(g), from 2-fluoro-4-pyridylboronic acid and 3-bromo-2-(4-fluorophenyl)-6-trifluoromethylpyrazolo[1,5-a]pyridine (Example 19(d)) was obtained the title compound. ¹H NMR (CDCl₃): δ 8.85(s, 1H), 8.22(d, 1H, J=5.2 Hz), 7.70 (d, 1H, J=9.6 Hz), 7.52(dd, 2H, J=5.2, 8.4 Hz), 7.38(d, 1H, 9.6 Hz), 7.09(t, 2H, J=8.4 Hz), 6.90(s, 1H). MS (ES+ve): 376 (100, M⁺).

b) In a similar manner as described in Example 1(h) using 2-(4-fluorophenyl)-3-(2-fluoro-4-pyridinyl)-6-trifluoromethylpyrazolo[1,5-a]pyridine and isopropylamine was obtained the title compound. ¹H NMR (d₆-acetone): δ 9.12(s, 1H), 8.04(d, 1H, J=5.1 Hz), 7.85(d, 1H, J=9.3 Hz), 7.70 (dd, 2H, J=5.4, 8.7 Hz), 7.50(d, 1H. J=9.3 Hz), 7.21(t, 2H, J=8.7 Hz), 6.49(s, 1H), 6.45(d, 1H, J=5.1 Hz), 5.63(m, 1H), 4.04(m, 1H), 1.20 (d, 6H, J=4.8 Hz). MS (ES+ve): 415 (100, M⁺).

### EXAMPLE 14

### 3-(4-[2-(4-Fluorophenyl)-6-trifluoromethylpyrazolo[1,5-a]-pyridin-3-yl]-2-pyridinylamino)-1-propanol

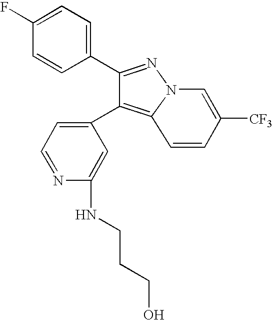

In a similar manner as described in Example 1(h) using 2-(4-fluorophenyl)-3-(2-fluoro-4-pyridinyl)-6-trifluoromethylpyrazolo[1,5-a]pyridine (Example 13) and 3-hydroxypropylamine was obtained the title compound. ¹H NMR (d₆-DMSO): δ 9.41(s, 1H), 7.95(d, 1H. J=5.2 Hz), 7.78(d, 1H, 9.2 Hz), 7.58(dd, 2H, J=5.6, 8.8 Hz), 7.50(d, 1H. J=9.6 Hz), 7.26(t, 2H, J=8.8 Hz), 6.544(m, 1H, J=5.6 Hz), 6.42(s, 1H), 6.33(d, 1H, J=5.6 Hz), 6.46(m, 1H), 3.43(m, 2H), 3.22(m, 2H, J=6.8 Hz), 1.62(quint, 2H, J=6.4 Hz). MS (ES+ve): 431(100, M⁺).

### EXAMPLE 15

### N-(3-Aminopropyl)-4-[6-bromo-2-(4-fluorophenyl)-pyrazolo[1,5-a]pyridin-3-yl]-2-pyridinamine

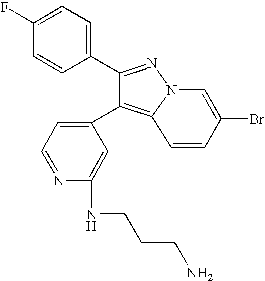

### a) 6-Bromo-2-(4-fluorophenyl)-3-(2-fluoro-4-pyridinyl)pyrazolo[1,5-a]pyridine

To a solution of 2-(4-fluorophenyl)-3-(2-fluoro-4-pyridinyl)pyrazolo[1,5-a]-pyridine (Example 1, 937 mg, 3.05 mmol) in DMF (20 mL) was added N-bromosuccinimide (651 mg, 3.66 mmol). The reaction mixture was heated at 60° C. for about 5 hours and then allowed to cool to room temperature. Saturated sodium bicarbonate was added and the mixture was extracted with dichloromethane. The organic extracts were dried over anhydrous magnesium sulfate and the solvents removed under vacuum. The residue was purified by silica gel chromatography to give the title compound 0.604 g (50%). ¹H NMR (CDCl₃): δ 8.68(s, 1H), 8.20 (d, 1H, J=5.4 Hz), 7.53 (m, 3H), 7.35 (dd, 1H, J=9.3, 1.2 Hz), 7.10 (m, 3H), 7.00(s, 1H). MS (ES+ve) 387 (50, M⁺, M+3).

b) In a similar manner as described in Example 1(h), using 6-bromo-2-(4-fluorophenyl)-3-(2-fluoro-4-pyridinyl)pyrazolo[1,5-a]pyridine and 1,3-diaminopropane was obtained the title compound. ¹H NMR (d₆-acetone): δ 8.94 (s, 1H), 8.06 (d, 1H, J=4.8 Hz), 7.72 (m, 3H), 7.44 (dd, 1H, J=1.5, 9.6 Hz), 7.23 (m, 3H), 6.51 (s 1H), 6.48 (dd, 1H, J=1.2, 6.3 Hz), 6.08 (m 1H), 3.44 (q, 2H, J=5.7 Hz), 3.31 (t, 2H, J=6.3 Hz), 1.90 (quint, 2H, J=6.8 Hz). MS (ES+ve) 440 (100, M⁺, M+3).

### EXAMPLE 16

### N-Butyl-4-[2-(4-fluorophenyl)pyrazolo[1,5-a]pyridin-3-yl]-2-pyrimidin-amine

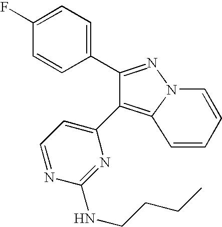

A solution of 2-(4-fluorophenyl)-3-(4-(2-methylsulfinyl)pyrimidinyl)-pyrazolo[1,5-a]pyridine (Example 17(c), 0.03 g, 0.085 mmol) in n-butylamine (0.5 mL) was heated to reflux for 0.25 h. On cooling a white solid deposits which was collected by filtration, washed with hexane and dried under vacuum to give the title compound as a white solid, 0.029 g (94%). ¹H NMR (DMSO-d₆): δ 0.87 (t, J=7.4 Hz, 3H), 1.31 (sextet, J=7.4 Hz, 2H), 1.49(quintet, J=7.2 Hz, 2H), 3.25 (q, J=6.6 Hz, 2H), 6.4 (broad s, 1H), 7.06 (t, J=6.8 Hz, 1H), 7.13 (broad s, 1H), 7.29 (t, J=8.8 Hz, 2H), 7.43 (t, J=7.8 Hz, 1H), 7.59 (dd, J=5.7, 8.5 Hz, 2H), 8.01 (d, J=5.3 Hz, 1H), 8.40 (broad s, 1H), 8.76 (d, J=6.9 Hz, 1H); APESI+MS m/z 362 (M+1)⁻.

### EXAMPLE 17

### 4-[2-(4-Fluorophenyl)pyrazolo[1,5-a]pyridin-3-yl]-N-(2-propyl)-2-pyrimidinamine

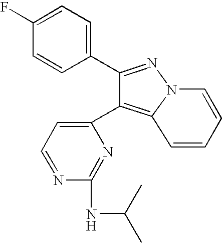

### a) 1-(4-Fluorophenyl)-2-(4-(2-methylthio)pyrimidinyl)ethanone

To a stirred solution of 2-methylthio-4-methylpyrimidine (66 g, 0.47 mol) and ethyl 4-fluorobenzoate (79 g, 0.47 mol) in dry THF (400 mL) at 0° C. under nitrogen was added lithium bis(trimethylsilyl)amide (1N in THF, 940 mL 0.94 mol) over a 2 h period. The solution was stirred at ice bath temperature for 18 h. The solution was poured into 2 L of ice cold 0.5 N HCl. A precipitate formed which was filtered off and air dried. Second and third crops of solids were obtained as the precipitate was washed with water. The combined precipitates were recrystalized from acetone and water to give product as a yellow solid: 117 g (95%). ¹H NMR (CDCl₃): δ (all in enol form): 3.0 (s, 3H), 6.29 (s, 1H), 7.01 (d, J=5.7 Hz, 1H), 7.48 (t, J=8.7 Hz, 2H), 8.20 (dd, J=5.4, 8.8 Hz, 2H), 8.68 (d, J=5.7 Hz, 1H); APESI−MS m/z 261 (M−1)⁻.

### b) 2-(4-Fluorophenyl)-3-(4-(2-methylthio)pyrimidinyl)-pyrazolo[1,5-a]pyridine

A solution of 1-(4-fluorophenyl)-2-(4-(2-methylthio)pyrimidinyl)ethanone (13.0 g, 50 mmol) in isopropanol (300 mL) was warmed to reflux. A solution of 1-aminopyridinium iodide (14 g, 63 mmol) in water (300 mL) was treated with 2N NaOH (31.5 mL). This solution was added to the ketone over a period of two hours while the mixture was heated at reflux. After an additional seven hours, the isopropanol was partially evaporated under reduced pressure and the resulting solution was extracted with dichloromethane (2×300 mL). The dichloromethane extracts were combined, dried (MgSO₄), filtered and the solvent evaporated under reduced pressure to leave a red solid which was purified by silica gel chromatography with dichloromethane to give the title compound as a yellow solid, 4.5 g (26%). ¹H NMR (DMSO-d₆): δ 2.5 (s, 3H), 6.80 (d, J=5.3 Hz, 1H), 7.18 (t, J=6.9 Hz, 1H), 7.36 (t, J=8.8 Hz, 2H), 7.59 (t, J=7.9 Hz, 1H), 7.60 (dd, J=5.7, 8.7 Hz, 2H), 8.38 (d, J=9.1 Hz, 1H), 8.40 (d, J=5.3 Hz, 1H), 8.88 (d, J=7.0 Hz, 1H), APESI+MS m/z 337 (M+1).

### c) 2-(4-Fluorophenyl)-3-(4-(2-methylsulfinyl)pyrimidinyl)pyrazolo[1,5-a]pyridine

To a stirred solution of 2-(4-fluorophenyl)-3-(4-(2-methylthio)pyrimidinyl)-pyrazolo[1,5-a]pyridine (0.285 g, 0.85 mmol) in dichloromethane (10 mL) was added, dropwise, a solution of (0.257 g, 0.85-1.23 mmol) of 57-86% m-chloroperoxybenzoic acid in dichloromethane (5 mL). After 10 min., the solution was quenched by the addition of aqueous potassium carbonate (20 mL), and the organic phase was separated. The aqueous phase was further extracted with dichloromethane (2×20 mL) and the dichloromethane phases dried over magnesium sulfate and concentrated to give a crude white solid. Chromatography on silica gel eluting with a hexane/EtOAc gradient (0-100% EtOAc) gave the title compound as a white solid, 0.213 g (60: ¹H NMR (CDCl₃): δ 3.05 (s, 3H), 7.07-7.11 (m, 2H), 7.25 (d, J=8.5 Hz, 2H), 7.55 (t, J=7.8 Hz, 1H), 7.64 (dd, J=5.5, 6.9 Hz, 2H), 8.52 (d, J=5.1 Hz, 1H), 8.59 (d, J=6.9 Hz, 1H), 8.84 (d, J=9.0 Hz, 1H); APESI+MS m/z 353 (M+1)⁻.

d) In a similar manner as described for Example 16, from 2-(4-fluorophenyl)-3-(4-(2-methylsulfinyl)pyrimidinyl)pyrazolo[1,5-a]pyridine (0.063 g, 0.18 mmol) and isopropylamine was obtained the title compound as a white solid, 0.022 g (66%). ¹H NMR (CDCl₃): δ 1.28 (d, J=6.6 Hz, 6H), 4.21 (septet, J=6.6 Hz, 1H), 5.02 (broad s, 1H), 6.29 (d, J=5.3 Hz, 1H), 6.89 (t, J=6.4 Hz, 1H), 7.12 (t, J=8.6 Hz, 2H), 7.31 (t, J=7.9 Hz, 1H), 7.60 (dd, J=5.5, 8.6 Hz, 2H), 8.03 (d, J=5.3 Hz, 1H), 8.38 (d, J=8.9 Hz, 1H), 8.48 (d, J=7.0 Hz, 1H); APESI+MS m/z 348 (M+1).

### EXAMPLE 18

### 4-[2-(4-Fluorophenyl)pyrazolo[1,5-a]pyridin-3-yl]-2-pyrimidinamine

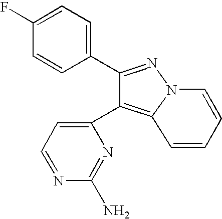

### a) 2-(4-Fluorophenyl)-3-acetylpyrazolo[1,5-a]pyridine

A mixture of 2-(4-fluorophenyl)pyrazolo[1,5-a]pyridine (2.00 g, 9.42 mmol) in acetic anhydride (20 mL) and conc. H₂SO₄ (2 drops) was stirred and heated at reflux for 30 min. The mixture was cooled to room temperature, poured into ice water (300 mL), and basified (pH=10) using 1N NaOH(aq). The resulting orange precipitate was collected by filtration, washed with water, air-dried, then dried under high-vacuum to afford the title compound as an orange solid, 2.60 g (quant.). ¹H NMR (CDCl₃) δ 8.56 (d, 1H, J=6.9 Hz), 8.45 (d, 1H, J=9.3 Hz), 7.62 (m, 2H), 7.54 (m, 1H), 7.24 (m, 2H), 7.08 (m, 1H), 2.20 (s, 3H). MS (+ve ion electrospray) 255 (100), (MH⁺).

### b) 2-(4-Fluorophenyl)-3-(3-(dimethylamino)-2-propenoyl)pyrazolo[1,5-a]pyridine

A mixture of 2-(4-fluorophenyl)-3-acetylpyrazolo[1,5-a]pyridine (1.0 g, 3.93 mmol) in N,N-dimethylformamide dimethyl acetal (10 mL) was stirred and heated at reflux for 17 hours. The mixture was cooled to room temperature and the volatiles evaporated under reduced pressure. The residue was purified by silica gel chromatography (eluded with 1% MeOH/CH₂Cl₂) to afford the title compound as an orange solid, 0.830 g (68%). ¹H NMR (CDCl₃) δ 8.50 (d, 1H, J=6.9 Hz), 8.39 (d, 1H, J=9.0 Hz), 7.83 (d, 2H, J=12.6 Hz), 7.73 (m, 2H), 7.39 (m, 1H), 7.20 (m, 2H), 6.93 (m, 1H), 5.13 (d, 1H, J=12.5 Hz), 3.10 (s, 3H), 2.56 (s, 3H). MS (+ve ion electrospray) 310 (90), (MH+).

### c) 4-[2-(4-Fluorophenyl)pyrazolo[1,5-a]pyridin-3-yl]-2-pyrimidinamine

A mixture of 2-(4-fluorophenyl)-3-(3-(dimethylamino)-2-propenoyl)pyrazolo[1,5-a]pyridine (60 mg, 0.19 mmol), guanidinium hydrochloride (36 mg, 0.38 mmol), and K₂CO₃ (105 mg, 0.76 mmol) in N,N-dimethylformamide (3 mL) was stirred in a 110° C. oil bath for 8 hours. Additional guanidinium hydrochloride (36 mg, 0.38 mmol) was added, and the mixture stirred in a 110° C. oil bath for 16 hours. The mixture was cooled to room temperature, and water (20 mL) added. The resulting tan precipitate was collected by filtration, washed with water, air-dried, then dried under high-vacuum to afford the title compound, 0.033 g (57%). ¹H NMR (CDCl₃) δ 8.57 (d, 1H, J=6.0 Hz), 8.51 (d, 1H, J=8.9 Hz), 7.98 (d, 2H, J=5.7 Hz), 7.64 (m, 2H), 7.46 (m, 1H), 7.22 (m, 2H), 7.04 (m, 1H), 6.47 (d, 1H, J=5.8 Hz), 5.76 (s, 2H). MS (+ve ion electrospray) 306 (100), (MH⁺).

### EXAMPLE 19

### 4-[2-(4-Fluorophenyl)-6-trifluoromethylpyrazolo[1,5-a]pyridin-3-yl]-2-pyrimidinamine

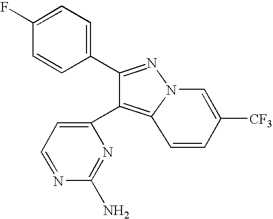

### a) 1-(4-Fluorophenyl)-2-(2-(5-trifluoromethyl)pyridyl)ethanone

To a solution of 4-fluoroacetophenone (13.8 g, 0.100 mol) and 2-chloro-5-trifluoromethylpyridine (20.0 g, 0.110 mol) in tetrahydrofuran (400 mL) was added sodium hydride (95%, 5.56 g, 0.220 mol) in several portions. The reaction was stirred at room temperature for 72 hours then carefully quenched by the addition of water (300 mL) and diethyl ether (200 mL). The organic layer was separated and extracted with 6N HCl (2×300 mL). The aqueous extracts were cooled to 0° C. and 6N NaOH was used to adjust the solution to pH12. The mixture was then extracted with diethyl ether and the combined organic extracts were dried over magnesium sulfate. The drying agent was removed by filtration and the filtrate was evaporated to dryness to afford the title compound as a tautomeric mixture, 20.9 g (73%). ¹H NMR (CDCl₃): δ 8.87(s), 8.63(s), 8.14(dd, J=5.1, 8.4 Hz), 8.00-7.83(m), 7.51(d, J=8.4 Hz), 7.22-7.12(m), 6.13(s), 4.60(s). MS (ES+ve): 284 (100, M⁺+1).

### b) 1-(4-Fluorophenyl)-2-(2-(5-trifluoromethyl) pyridyl)ethanone Oxime

To a solution of 1-(4-fluorophenyl)-2-(2-(5-trifluoromethyl)pyridyl)ethanone (80.0 g, 0.282 mol) in methanol (1 L) at room temperature was added 10% aqueous sodium hydroxide (436 mL, 1.09 mol). The resulting solution was stirred vigorously as solid hydroxylamine hydrochloride (98.0 g, 1.40 mol) was added. The mixture was heated to reflux for 2 hours, treated with decolorizing charcoal while hot, then filtered through Celite while hot. The filtrate was concentrated to one-half its original volume and then cooled to 0° C. with stirring for one hour. The resulting solids were collected by filtration, washed with water, and dried under vacuum at 50° C. overnight to provide the title compound as a light yellow powder, 73.9 g (88%). ¹H NMR (DMSO-d₆): δ11.60(s, 1H), 8.86(s, ₁H), 8.14(dd, ₁H, J=2.1, 8.1 Hz), 7.78(dd, 2H, J=5.7, 9.0 Hz), 7.53(d, 1H, J=8.4 Hz), 7.23(t, 2H, J=9.0 Hz), 4.40(s, 2H). MS (ES+ve): 299 (70, M⁺+1).

### c) 3-(4-Fluorophenyl)-2-(2-(5-trifluoromethyl)pyridyl)-2H-azirine

To a solution of 1-(4-fluorophenyl)-2-(2-(5-trifluoromethyl)pyridyl)ethanone oxime (25.0 g, 0.084 mol) in methylene chloride (400 mL) was added triethylamine (46.7 mL, 0.335 mol). The solution was cooled to 0° C. under a nitrogen atmosphere, and trifluoroacetic anhydride (14.1 mL, 0.100 mol) was added dropwise. The reaction was stirred for 0.5 hours then quenched with water. The organic layer was separated and dried over anhydrous magnesium sulfate. The drying agent was removed by filtration and the solvent was evaporated from the filtrate to leave an oil. The residue was loaded onto a silica gel column and eluted with 15% ethyl acetate in hexanes to give the title compound as an oil which solidified on standing, 19.4 g (82%). ¹H NMR (CDCl₃): δ 8.76(s, 1H), 7.93(dd, 2H, J=5.4, 8.7 Hz), 7.83(dd, 1H, J=2.1, 8.4 Hz), 7.27(t, 2H, J=8.7 Hz), 7.21(d, 1H, J=8.1 Hz), 3.54 (s, 1H). MS (ES+ve): 281 (100, M⁺+1).

### d) 2-(4-Fluorophenyl)-6-trifluoromethylpyrazolo[1,5-a]pyridine

3-(4-Fluorophenyl)-2-(2-(5-trifluoromethyl) pyridyl)-2H-azirine (40.0 g, 0.143 mol) was dissolved in 1,2,4-trichlorobenzene (400 mL) and the mixture was heated to 200° C. for 10 hours. The reaction mixture was then cooled to room temperature and poured onto a silica gel column. The column was eluted with hexanes to remove the 1,2,4-trichlorobenzene, and then with 20% diethyl ether in hexanes to elute the product. The desired fractions were combined and the solvent was evaporated under reduced pressure to leave the title compound, 28.7 g (71%). ¹H NMR (CDCl₃): δ 8.84(s, 1H), 7.98(dd, 2H, J=5.4, 8.7 Hz), 7.65(d, 1H. J=9.3 Hz), 7.28(d, 1H, J=9.3 Hz), 7.20(t, 2H, J=8.7 Hz), 6.88(s, 1H). MS (ES+ve): 281 (100, M⁺+1).

### e) 2-(4-Fluorophenyl)-3-acetyl-6-trifluoromethylpyrazolo[1,5-a]pyridine

To a mixture of 2-(4-fluorophenyl)-6-trifluoromethylpyrazolo[1,5-a]pyridine (10.30 g, 36.76 mmol) and acetic anhydride (100 mL) was added conc. sulfuric acid (10 drops) and the mixture was stirred and heated at reflux for 1 hour. The reaction mixture was cooled to room temperature and poured into ice water (300 mL). 2N Aqueous sodium hydroxide solution was added to raise the pH of the solution to about 10 and the resulting orange precipitate was collected by filtration. The solid was washed with water, air-dried, and then dried under vacuum to afford the title compound as an orange solid, 11.87 g (quant). ¹H NMR (DMSO-d6): δ 9.58 (s, 1H), 8.41 (d, 1H, J=9.3 Hz), 7.89 (d, 1H, J=9.5 Hz), 7.74 (m, 2H), 7.39 (m, 2H), 2.22 (s, 3H). MS (+ve ion electrospray) 323 (70), (MH⁺).

### f) 2-(4-fluorophenyl)-3-(3-(dimethylamino)-2-propenoyl)-6-trifluoromethyl-pyrazolo[1,5-a]pyridine

A mixture of 2-(4-fluorophenyl)-3-acetyl-6-trifluoromethylpyrazolo[1,5-a]pyridine (11.85 g, 36.77 mmol) and N,N-dimethylformamide dimethyl acetal (100 mL) was stirred at reflux for 17 hours. The mixture was cooled to room temperature and then to 0° C. The resulting orange precipitate was collected by filtration, washed with cold hexanes, and dried under vacuum to afford the title compound as an orange solid, 10.17 g (73%). ¹H NMR (DMSO-d6): δ 9.44 (s, 1H), 8.22 (d, 1H, J=9.4 Hz), 7.75 (m, 2H), 7.65 (d, 1H, J=9.5 Hz), 7.56 (d, 1H, J=12.4 Hz), 7.35 (m, 2H), 5.05 (d, 1H, J=12.3 Hz), 3.04 (s, 3H), 2.56 (s, 3H). MS (+ve ion electrospray) 377 (80), (M+).

### g) 4-[2-(4-Fluorophenyl)-6-trifluoromethylpyrazolo[1,5-a]pyridin-3-yl]-2-pyrimidinamine

A mixture of 2-(4-fluorophenyl)-3-(3-(dimethylamino)-2-propenoyl)-6-trifluoromethylpyrazolo[1,5-a]pyridine (100 mg, 0.27 mmol), guanidinium hydrochloride (52 mg, 0.54 mmol), and sodium ethoxide (73 mg, 1.08 mmol) in EtOH (4 mL) was stirred at reflux for 21 hours. Additional guanidine was added in portions to the mixture until starting material was consumed as evidenced by TLC. The reaction mixture was cooled to 0° C. and the resulting precipitate was collected by filtration, washed with cold EtOH and dried under vacuum to afford the title compound as a tan solid, 93 mg (92%). ¹H NMR (CD₃COCD₃): δ 9.19 (s, 1H), 8.73 (d, 1H, J=9.4 Hz), 8.13 (d, 1H, J=5.2 Hz), 7.78 (m, 2H), 7.63 (d, 1H, J=9.5 Hz), 7.34 (m, 2H), 6.41 (d, 1H, J=5.2 Hz), 6.17 (s, 1H). MS (+ve ion electrospray) 374 (100), (MH⁺).

### EXAMPLE 20

### N-Benzyl-4-[2-(4-fluorophenyl)-6-trifluoromethylpyrazolo[1,5-a]pyridin-3-yl]-2-pyrimidinamine

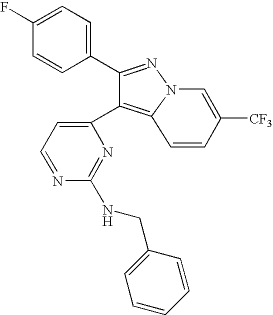

In a similar manner as described for Example 19g, using N-benzylguanidine in place of guanidinium hydrochloride was obtained the title compound as a tan solid, (quant.). ¹H NMR (CD₃COCD₃): δ 9.09 (s, 1H), 8.12 (d, 1H, J=5.1 Hz), 7.69 (m, 2H), 7.24-7.42 (m, 7H), 7.01 (m, 1H), 6.34 (d, 1H, J=5.1 Hz), 4.70 (d, 2H, J=6.2 Hz). MS (+ve ion electrospray) 464 (95), (MH⁺).

### EXAMPLE 21

### N-Cyclopropyl-4-[2-(4-fluorophenyl)-6-trifluoromethylpyrazolo[1,5-a]pyridin-3-yl]-2-pyrimidinamine

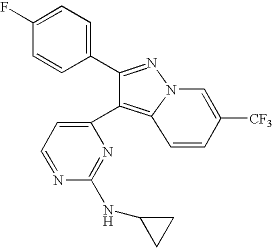

In a similar manner as described for Example 19g, using N-cyclopropylguanidine in place of guanidinium hydrochloride was obtained the title compound as an off-white solid, (77%). ¹H NMR (CD₃COCD₃): δ 9.14 (s, 1H), 8.88 (s, 1H), 8.11 (d, 1H, J=5.0 Hz), 7.73 (m, 2H), 7.62 (d, 1H. J=9.4 Hz), 7.30 (m, 2H), 6.62 (s, 1H), 6.37 (s, 1H, J=5.1 Hz), 2.87 (m, 1H), 0.80 (m, 2H), 0.60 (m, 2H). MS (+ve ion electrospray) 414 (100), (MH⁺).

### EXAMPLE 22

### 4-[2-(4-Fluorophenyl)-6-trifluoromethylpyrazolo[1,5-a]pyridin-3-yl]-N-(2,2,2-trifluoroethyl)-2-pyrimidinamine

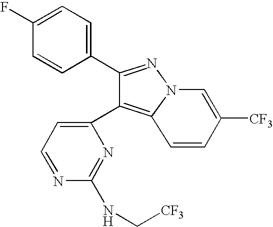

In a similar manner as described for Example 19g, using N-(2,2,2-trifluoroethyl)guanidine in place of guanidinium hydrochloride was obtained the title compound as a white solid, (2401%). ¹H NMR (CD₃COCD₃): δ 9.16 (s, 1H), 8.62 (s, 1H), 8.19 (d, 1H, J=5.0 Hz), 7.71 (m, 2H), 7.61 (d, 1H, J=9.3 Hz), 7.28 (m, 2H), 7.03 (s, 1H), 6.51 (d, 1H, J=4.0 Hz), 4.28 (m, 2H). MS (+ve ion electrospray) 456 (100), (MH+).

### EXAMPLE 23

### 3-(4-[2-(4-Fluorophenyl)-6-trifluoromethylpyrazolo[1,5-a]pyridin-3-yl]-2-pyrimidinylamino)-1-propanol

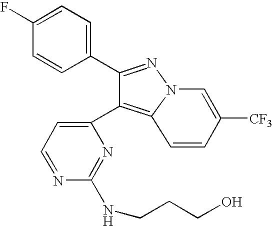

### a) 4-[2-(4-Fluorophenyl)-6-trifluoromethylpyrazolo[1,5-a]pyridin-3-yl]-N-(3-(4-methoxybenzyloxy)propyl)-2-pyrimidinamine

A mixture of 2-(4-fluorophenyl)-3-(3-(dimethylamino)-2-propenoyl)-6-trifluoromethyl-pyrazolo[1,5-a]pyridine (Example 19(f) 2.0 g, 5.3 mmol), N-(3-(4-methoxybenzyloxy)-propyl)guanidine (2.7 g, 7.95 mmol), and potassium carbonate (2.2 g, 15.9 mmol) was stirred in N,N-dimethylformamide (20 mL) in a 100° C. oil bath for 18 hours. The mixture was cooled to room temperature, water (200 mL) was added the mixture was extracted with chloroform. The chloroform extracts were dried over anhydrous MgSO₄, filtered, and the solvent was evaporated. The crude material was purified by chromatography on silica gel using 30% EtOAc/hexanes as eluent to afford the title compound as a white solid, 2.1 g (720/%). ¹H NMR (CD₃COCD₃): δ 9.18 (s, 1H), 8.67 (d, 1H, J=9.4 Hz), 8.15 (d, 1H, J=5.1 Hz), 7.77 (m, 2H), 7.56 (d, 1H, J=9.2 Hz), 7.34 (m, 4H), 6.90 (d, 2H, J=8.6 Hz), 6.50 (s, 1H), 6.38 (d, 1H, J=5.1 Hz), 4.49 (s, 2H), 3.80 (s, 3H), 3.63 (m, 4H), 1.98 (m, 2H). MS (+ve ion electrospray) 551 (30), (M+).

### b) 3-(4-[2-(4-Fluorophenyl)-6-trifluoromethylpyrazolo[1,5-a]pyridin-3-yl]-2-pyrimidinylamino)-1-propanol

A solution of 4-[2-(4-fluorophenyl)-6-trifluoromethylpyrazolo[1,5-a]pyridin-3-yl]-N-(3-(4-methoxybenzyloxy)propyl)-2-pyrimidinamine (2.1 g, 3.8 mmol) in 4N HCl/dioxane (5 mL) was stirred at room temperature for 4.5 hours, then heated to reflux for 1 hour. The mixture was cooled to room temperature, neutralized with saturated aqueous NaHCO₃, and extracted with EtOAc. The EtOAc extracts were dried over anhydrous MgSO₄, filtered, and the solvent was evaporated. The residue was triturated with 2% EtOAc/hexanes to afford a solid which was collected by filtration and dried to give the title compound as a white solid, 1.31 g (80% yield). ¹H NMR (CD₃COCD₃): δ 9.20 (s, 1H), 8.73 (d, 1H, J=9.3 Hz), 8.15 (d, 1H, J=5.1 Hz), 7.77 (m, 2H), 7.64 (d, 1H, J=9.9 Hz), 7.34 (m, 2H), 6.50 (s, 1H), 6.40 (d, 1H, J=5.1 Hz), 3.60-3.70 (m, 4H), 1.88 (m, 2H). MS (+ve ion electrospray) 432 (95), (MH+).

### EXAMPLE 24

### N-Cyclopropyl-4-[6-cyano-2-(4-fluorophenyl)pyrazolo[1,5-a]pyridin-3-yl]-2-pyrimidinamine

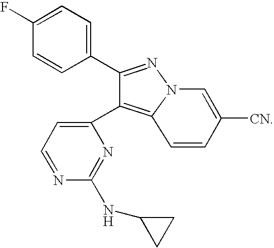

### a) 2-(2-(5-Cyanopyridyl))-1-(4-fluorophenyl)ethanone

To a cooled solution (0° C.) of 6-methylnicotinonitrile (5.0 g, 42 mmol) and ethyl 4-fluorobenzoate (6.2 mL, 42 mmol) in anhydrous tetrahydrofuran (50 mL) under N₂ was added lithium bis(trimethylsilyl)amide (1.0M solution in tetrahydrofuran. 84 mL, 84 mmol). The reaction mixture was warmed to room temperature and was allowed to stir at room temperature for 18 hours. The solvents were evaporated under reduced pressure and the residue was triturated with ether and water. The resulting solid was collected by filtration and dried in vacuo to give the title compound as a yellow solid, 10.2 g (quant.). ¹H NMR (400 MHz, d₆-DMSO) showed a mixture of tautomers.

### b) 2-(4-Fluorophenyl)-6-cyanopyrazolo[1,5-a]pyridine

N-Boc-O-mesitylsulfonylhydroxylamine (26.7 g, 84.5 mmol) was added in portions to trifluoroacetic acid at 0° C. The mixture was stirred at 0° C. for 30 minutes and then poured into ice water. The resulting white precipitate was collected by filtration, washed with cold water, and dissolved in dichloromethane (300 mL). The organic solution was dried over anhydrous MgSO₄. The drying agent was removed by filtration and the filtrate was transferred to a flask. To this solution was added 2-(2-(5-cyanopyridyl))-1-(4-fluorophenyl)ethanone (6.77 g, 28.2 mmol) and the reaction mixture was stirred at room temperature for about 24 hours. The reaction mixture was washed with water, dried over MgSO₄, filtered through a short pad of silica gel and the solvent evaporated under reduce pressure. The residue was purified using chromatography to give the title compound as a brown solid, 2.6 g (39%). ¹H NMR (400 MHz, CDCl₃): δ 6.90 (s, 1H); 7.15, (m, 3H), 7.57 (d, 1H, J=8.0 Hz), 7.93 (dd, 2H, J=5.2, 8.4 Hz), 8.82 (s, 1H).

### c) 2-(4-Fluorophenyl)-3-acetyl-6-cyanopyrazolo[1,5-a]pyridine

A solution of 2-(4-fluorophenyl)-6-cyanopyrazolo[1,5-a]pyridine (6.7 g, 11 mmol) and concentrated sulfuric acid (2 drops) in acetic anhydride (25 mL) was heated, and stirred, at 120° C. under N₂ for 5 hours. The solution was cooled to room temperature, diluted with ice water and basified to pH 11 using 2 N aqueous sodium hydroxide. The solution was extracted with chloroform (3×), and the combined organic extracts were dried and the solvent was evaporated in vacuo. Trituration with methanol afforded a light brown solid which was collected and dried to give the title compound, 1.6 g (840/%). ¹H NMR (400 MHz, d₆-DMSO) δ 2.19 (s, 3H), 7.35 (t, 2H, J=8.0 Hz), 7.69 (dd, 2H, J=4.0, 8.0 Hz), 7.86 (dd, 1H, J=4.0, 16 Hz), 8.30 (d, 1H, J=12 Hz), 9.75 (s, 1H). MS (ES+) m/z 280 (M⁺+H).

### d) 2-(4-Fluorophenyl)-3-(3-(dimethylamino)-2-propenoyl)-6-cyanopyrazolo[1,5-a]pyridine

A mixture of 2-(4-fluorophenyl)-3-acetyl-6-cyanopyrazolo[1,5-a]pyridine (1.6 g, 5.6 mmol) and dimethylformamide-dimethylacetal (15 mL) was stirred and heated at 130° C., under N₂, overnight. The solution was cooled and the resulting solid was collected by filtration and rinsed with acetone. The filtrate was evaporated and the resulting solid was purified using chromatography. The product solids were combined to afford the title compound as a brown solid, 1.3 g (68%). ¹H NMR (300 MHz, d₆-DMSO) showed a mixture of isomers. MS (ES+) m/z 335 (M⁺+H), 264 (M⁺−70).

### e) N-Cyclopropyl-4-[6-cyano-2-(4-fluorophenyl)pyrazolo[1,5-a]pyridin-3-yl]-2-pyrimidinamine

To a solution of 2-(4-fluorophenyl)-3-(3-(dimethylamino)-2-propenoyl)-6-cyanopyrazolo[1,5-a]pyridine (1.3 g, 3.9 mmol) in dimethylformamide (20 mL), under N₂, was added N-cyclopropylguanidine (0.78 g, 7.8 mmol) and potassium carbonate (1.1 g, 7.8 mmol). The mixture was stirred and heated at 100° C. for 17 hours and then additional N-cyclopropyl-guanidine (0.39 g, 3.9 mmol) and potassium carbonate (0.55 g, 3.9 mmol) were added. The mixture was heated at 100° C. for an additional 4 hours and then the reaction mixture was cooled and water added. The resulting solid was collected by filtration. This solid was dissolved in diethyl ether and purified using chromatography to give the title compound as a yellow solid, 0.39 g (28%). ¹H NMR (400 MHz, d₆-DMSO): δ 0.50 (m, 2H), 0.69 (d, 2H, J=4.0 Hz), 2.69 (m, 1H), 6.29 (d, 1H, J=8.0 Hz), 7.34 (t, 2H, J=8.0 Hz), 7.47 (d, 1H, J=4.0 Hz), 7.69 (m, 3H), 8.11 (d, 1H, J=4.0 Hz), 8.56 (s, 1H) MS (ES+) m/z 370 (M⁺+H).

### EXAMPLE 25

### 2-(4-Fluorophenyl)-3-(4-(2-(3-hydroxypropyl)amino)pyrimidinyl)-6-pyrazolo-[1,5-a]pyridinylcarboxamide

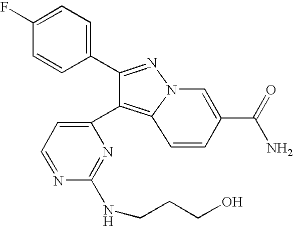

A solution of N-(3-hydroxypropyl)guanidine (5.4 mmol) (prepared from O-methylisourea-hydrochloride (0.597 g, 5.4 mmol) and propanolamine (0.405 g, 5.4 mmol)) in ethanol (15 mL) was added to a solution of sodium ethoxide (20 mmol) in ethanol (40 mL). To this mixture was added the enamine described in Example 19f (1.88 g, 5.0 mmol) and the reaction mixture was heated at reflux for 24 hours. The solvent was evaporated under reduced pressure and the residue was partitioned between saturated ammonium chloride solution and 2:1 ethyl acetate:diethyl ether.

The organic phase was dried over anhydrous magnesium sulfate, filtered to remove the drying agent and the solvents were evaporated. The resulting oil was purified by silica gel chromatography using 90% ethyl acetate in hexanes as eluent to give a pyrimidine orthoester compound 1.70 g (3.3 mmol). The orthoester described above (1.73 g, 3.40 mmol) was dissolved in acetone (200 mL) containing water (5 mL). To this solution was added para-toluenesulfonic acid (p-TSA) monohydrate (0.645 g, 3.40 mmol) and the reaction was stirred at room temperature for 30 minutes. The acetone was removed under reduced pressure and the residue was dissolved in a tetrahydrofuran:ethyl ether mixture (3:1). The organic phase was washed with saturated sodium bicarbonate solution. The organic layer was dried over anhydrous magnesium sulfate, filtered and concentrated to dryness. The residue was triturated with diethyl ether and the solids were collected by filtration to afford an ethyl ester, 0.965 g (2.20 mmol) as a white solid. A mixture of the ester described above (1.46 g, 2.98 mmol), sodium cyanide (15 mg, 0.30 mmol) and ammonia in methanol (30 mL, 7M solution) was stirred at room temperature for 5 days. Water (20 mL) was added and the mixture was stirred in an ice-water bath for 30 minutes. The resulting solid was collected by filtration and dried under vacuum. The solids were then triturated with tetrahydrofuran at 50° C. for 10 minutes, collected by filtration and dried under vacuum to afford the title compound, 0.935 g (2.30 mmol, 77% yield) as a white powder. NMR (d₆-DMSO, 80° C.): δ 9.30 (s, 1H), 8.44 (d, 1H, J=9.3 Hz), 8.11 (d, 1H, J=5.1 Hz), 7.87 (d, 1H, J=9.3 Hz), 7.6-7.75 (m, 3H), 7.32 (t, 2H, J=9 Hz), 6.85 (m, 1H), 6.30 (d, 1H, J=5.1 Hz), 4.25 (m, 1H), 3.56 (m, 2H), 3.43 (q, 2H, J=6.3 Hz), 1.77(pent, 2H, J=6.3 Hz).

Mass (ES+)=407 (1000/%).

### EXAMPLE 26

### 4-[2-(4-Fluorophenyl)pyrazolo[1,5-a]pyridin-3-yl]-N-[2-(1H-imidazol-5-yl)ethyl]-2-pyrimidinamine

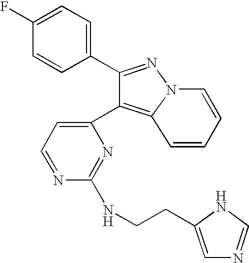

A solution of 2-(4-fluorophenyl)-3-(4-(2-methylsulfinyl)pyrimidinyl)-pyrazolo[1,5-a]pyridine (Example 17(c) 0.105 g, 0.31 mmol) and histamine (0.037 g, 0.33 mmol) in xylene (3 mL) was heated at 135° C. for 3 hours. The solvent was evaporated and the residue was purified on silica using methanol/ethyl acetate as eluent to give the title compound as a white solid, 0.044 g (33%).2. ¹H NMR (DMSO-d₆): δ 2.76 (t, J=7.1 Hz, 2H), 3.49 (d, J=6.9 Hz, 2H), 6.17 (d, J=4.4 Hz, 1H), 6.8 (broad s, 1H), 7.06 (t, J=6.8 Hz, 1H), 7.17 (broad s, 1H), 7.29 (t, J=8.8 Hz, 2H), 7.41 (t, J=7.9 Hz, 1H), 7.51 (s, 1H), 7.60 (dd, J=5.6, 8.6 Hz, 2H), 8.03 (d, J=5.1 Hz, 1H), 8.45 (broad s, 1H), 8.76 (d, J=6.9 Hz, 1H), 11.8 (broad s, 1H); APESI+MS m/z 400 (M+1)⁻.

### EXAMPLE 27

### 4-[2-(4-Fluorophenyl)pyrazolo[1,5-a]pyridin-3-yl]-N-(3-pyridinylmethyl)-2-pyrimidinamine

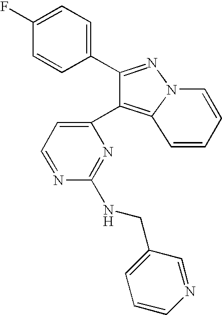

In a similar manner as described for Example 16, from 2-(4-fluorophenyl)-3-(4-(2-methylsulfinyl)pyrimidinyl)pyrazolo[1,5-a]pyridine (Example 17(c) 0.083 g, 0.25 mmol) and 3-aminomethylpyridine was obtained the title compound as a white solid, 0.071 g (72%). ¹H NMR (CDCl₃): δ 4.72 (d, J=6.1 Hz, 2H), 5.59 (broad s, 1H), 6.38 (d, J=5.4 Hz, 1H), 6.86 (t, J=6.8 Hz, 1H), 7.12 (t, J=8.7 Hz, 2H), 7.18 (t, J=7.6 Hz, 1H), 7.27 (dd, J=4.9, 7.7 Hz, 1H), 7.58 (dd, J=5.5, 8.4 Hz, 2H), 7.72 (d, J=7.6 Hz, 1H), 8.02 (broad s, 1H), 8.06 (d, J=5.3 Hz, 1H), 8.45 (d, J=6.8 Hz, 1H), 8.53 (d, J=4.6 Hz, 1H), 8.66 (s, 1H); APESI+MS m/z 397 (M+1)⁻.

### EXAMPLE 28

### 4-[2-(4-Fluorophenyl)pyrazolo[1,5-a]pyridin-3-yl]-N-(2-pyridinylmethyl)-2-pyrimidinamine

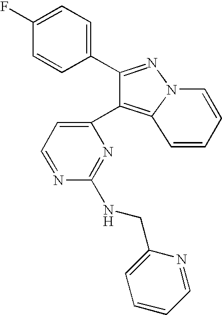

In a similar manner as described for Example 16, from 2-(4-fluorophenyl)-3-(4-(2-methylsulfinyl)pyrimidinyl)pyrazolo[1,5-a]pyridine (Example 17(c), 0.085 g, 0.25 mmol) and 2-aminomethylpyridine was obtained the title compound as a white solid, 0.047 g (47%). ¹H NMR (CDCl₃): δ 4.82 (d, J=5.7 Hz, 2H), 6.13 (broad s, 1H), 6.35 (d, J=5.3 Hz, 1H), 6.87 (t, J=6.7 Hz, 1H), 7.12 (t, J=8.6 Hz, 2H), 7.18-7.23 (m, 2H), 7.36 (d, J=7.8 Hz, 1H), 7.59 (dd, J=5.5, 8.6 Hz, 2H), 7.65 (dt, J=1.6, 7.7 Hz, 1H), 8.07 (d, J=5.3 Hz, 1H), 8.18 (broad s, 1H), 8.46 (d, J=7.0 Hz, 1H), 8.60 (d, J=4.9 Hz, 1H); APESI+MS m/z 397 (M+1).

### EXAMPLE 29

### 4-[2-(4-Fluorophenyl)pyrazolo[1,5-a]pyridin-3-yl]-N-(4-pyridinylmethyl)-2-pyrimidinamine

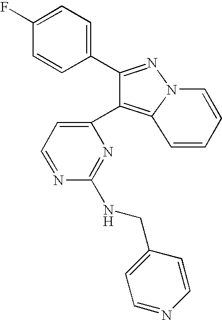

In a similar manner as described for Example 16, from 2-(4-fluorophenyl)-3-(4-(2-methylsulfinyl)pyrimidinyl)pyrazolo[1,5-a]pyridine (Example 16(c)) and 4-aminomethylpyridine was obtained the title compound as a white solid, (80%). ¹H NMR (CDCl₃): δ 4.71 (d, J=6.2 Hz, 2H), 5.69 (broad s, 1H), 6.38 (d, J=5.3 Hz, 1H), 6.85 (t, J=6.8 Hz, 1H), 7.11 (t, J=8.6 Hz, 3H), 7.33 (d, J=5.5 Hz, 2H), 7.58 (dd, J=5.5, 8.6 Hz, 2H), 7.8 (broad s, 1H), 8.06 (d, J=5.3 Hz, 1H), 8.45 (d, J=6.9 Hz, 1H), 8.58 (d, J=5.9 Hz, 2H); APESI+MS m/z 397 (M+1)⁻.

### EXAMPLE 30

### 4-[2-(4-Fluorophenyl)pyrazolo[1,5-a]pyridin-3-yl]-N-pentyl-2-pyridinamine

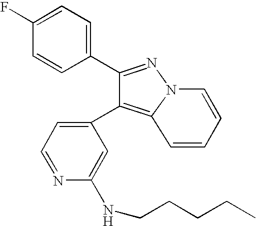

In a similar manner as described in Example 1(h), using pentylamine in place of histamine, was obtained the title compound. ¹H NMR (acetone-d₆): δ 8.66 (d, 1H, J=6.9 Hz); 8.05 (d, 1H, J=5.1 Hz), 7.73 (m, 3H), 7.65 (t, 2H, J=9.0 Hz), 7.22 (t, 2H, J=2.1 Hz), 7.02 (td, 1H, J=6.9, 1.2 Hz), 6.51 (s, 1H), 6.50 (d, 1H, J=5.4 Hz), 5.82 (m, 1H), 3.34 (quart, 2H, J=6.3 Hz), 1.63 (quint, 2H, J=6.9 Hz), 1.39 (m, 4H), 0.94 (t, 3H, J=6.3 Hz).

### EXAMPLE 31

### N-Butyl-4-[2-(4-fluorophenyl)-6-trifluoromethylpyrazolo-[1,5-a]pyridin-3-yl]-2-pyrimidinamine

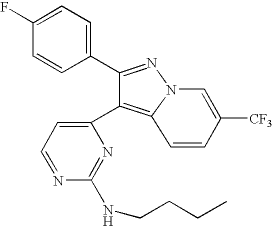

In a similar manner as described for Example 19, using N-butylguanidine in place of guanidinium hydrochloride was obtained the title compound as a yellow solid, (37%). ¹H NMR (CD₃COCD₃): δ 9.14 (s, 1H), 8.63 (d, 1H, J=9.3 Hz), 8.09 (d, 1H, J=5.1 Hz), 7.72 (m, 2H), 7.59 (d, 1H, J=9.3 Hz), 7.27 (m, 2H), 6.40 (s, 1H), 6.33 (d, 1H, J=4.2 Hz), 3.44 (m, 2H), 1.62 (m, 2H), 1.42 (m, 2H), 0.93 (m, 3H). MS (+ve ion electrospray) 430 (95), (MH+).

### EXAMPLE 32

### N-{4-[2-(4-fluorophenyl)-6-(trifluoromethyl)pyrazolo-[1,5-a]pyridin-3-yl]pyrimidin-2-yl}-N-[3-(4-methylpiperazin-1-yl)propyl]amine

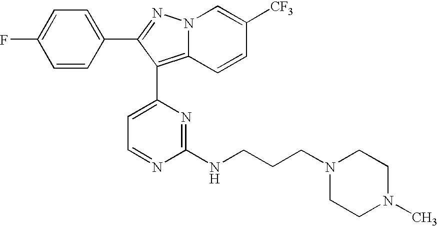

To a mixture of the enamine described in Example 19(f) (5.45 g, 14.45 mmol) and N-(3-(4-methylpiprazino)propyl)guanidine hydrogen sulfate (12.88 g, 3.0 equiv, 43.4 mmol) in anhydrous DMF (50 mL) under nitrogen was added powdered K₂CO₃ (2.75 g, 5.0 equiv, 20.0 mmol). The mixture was stirred and heated at 130° C. for 37 h and then filtered through a glass fritted funnel while warm. The solvent was evaporated under reduced pressure and the residue was triturated with EtOAc/Hexanes (1:10) to afford a solid that was collected by filtration and dried under vacuum to give the desired product as an off-white solid, 5.0 g (67%). ¹H NMR (300 MHz, CDCl₃): δ 1.85 (m, 2H), 2.30 (s, 3H), 2.53 (m, 10H), 3.54 (m, 2H), 6.00 (s, 1H), 6.30 (d, 1H), 7.14 (m, 2H), 7.40 (d, 1H), 7.60 (m, 2H), 8.08 (d, 1H), 8.49 (d, 1H), 8.81 (s, 1H). MS (ESI+) m/z 514.19 (M+H).

### EXAMPLE 33

### [3-[2-(Cyclopentylamino)-4-pyrimidinyl]-2-(4-fluorophenyl)pyrazolo-[1,5-a]pyridin-6-yl]methanol

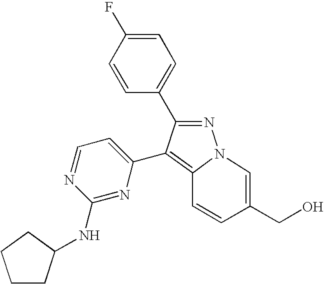

### a) 2-(4-Fluorophenyl)-6-(trifluoromethyl)pyrazolo[1,5-a]pyridine-3-carbaldehyde

To a cold (0° C.) solution of phosphorus oxychloride (8.0 mL, 86 mmol) in N,N-dimethylformamide (160 mL) was added 2-(4-fluorophenyl)-6-(trifluoromethyl)pyrazolo[1,5-a]pyridine (11.0 g, 39.3 mmol). The reaction mixture was stirred at room temperature for 72 hours, then quenched with ice water. The solid precipitate was collected on a filter to provide 2-(4-fluorophenyl)-6-(trifluoromethyl)pyrazolo[1,5-a]pyridine-3-carbaldehyde (11.4 g, 94%) as a white solid. R_{f} 0.45 (4:1 hexanes:ethyl acetate); ¹H NMR (400 MHz, CDCl₃) δ 10.15 (s, 1H), 8.92 (s, 1H), 8.53 (d, 1H), 7.80 (m, 2H), 7.70 (d, 1H), 7.27 (t, 2H).

### b) 1-[2-(4-Fluorophenyl)-6-(trifluoromethyl)pyrazolo[1,5-a]pyridin-3-yl]-2-propyn-1-ol

To a cold (−78° C.) suspension of 2-(4-fluorophenyl)-6-(trifluoromethyl)pyrazolo[1,5-a]pyridine-3-carbaldehyde (11.4 g, 37.0 mmol) in tetrahydrofuran (100 mL) was added ethynylmagnesium bromide (111 mL, 0.5 M in tetrahydrofuran, 56 mmol). The reaction mixture was warmed to room temperature and stirred for 14 hours. The reaction mixture was poured into water and adjusted to neutral pH with 1N aqueous hydrochloric acid. The aqueous mixture was extracted with ethyl acetate. The combined extracts were washed with water and brine. The organic layer was dried over magnesium sulfate. Filtration and concentration provided 1-[2-(4-fluorophenyl)-6-(trifluoromethyl)pyrazolo[1,5-a]pyridin-3-yl]-2-propyn-1-ol (11.9 g, 96%) as a tan solid. R_{f} 0.18 (4:1 hexanes:ethyl acetate); ¹H NMR (300 MHz, CDCl₃) δ 8.81 (s, 1H), 8.15 (d, 1H), 7.75 (m, 2H), 7.35 (d, 1H), 7.19 (t, 2H), 5.76 (s, 1H), 2.71 (d, 1H), 2.60 (d, 1H); MS m/335 (M+1).

### c) 1-[2-(4-Fluorophenyl)-6-(trifluoromethyl)pyrazolo[1,5-a]pyridin-3-yl]-2-propyn-1-one

To a cold (0° C.) solution of 1-[2-(4-fluorophenyl)-6-(trifluoromethyl)pyrazolo[1,5-a]pyridin-3-yl]-2-propyn-1-ol (5.00 g, 15.0 mmol) in chloroform (400 mL) was added manganese dioxide (130 g, 1.50 mol). The reaction mixture was stirred at 0° C. for 1.5 hours. The reaction mixture was filtered through a pad of Celite. The filtrate was concentrated in vacuo to provide 1-[2-(4-fluorophenyl)-6-(trifluoromethyl)pyrazolo-[1,5-a]pyridin-3-yl]-2-propyn-1-one (3.44 g, 69%) as a clear oil. R_{f} 0.39 (4:1 hexanes:ethyl acetate); ¹H NMR (400 MHz, CDCl₃) δ 8.90 (s, 1H), 8.61 (d, 1H), 7.72-7.69 (m, 3H), 7.17 (m, 2H), 3.06 (s, 1H); MS m/z 333 (M+1).

### d) N-Cyclopentyl-4-[2-(4-fluorophenyl)-6-(trifluoromethyl)pyrazolo[1,5-a]pyridin-3-yl]-2-pyrimidinamine

To a suspension of N-cyclopentylguanidine hydrochloride (2.20 g, 13.5 mmol) in ethanol (70 mL) was added sodium ethoxide (4.5 mL, 3 M in ethanol, 14 mmol). The mixture was stirred at room temperature for 30 minutes, then cooled to 0° C. To this mixture was added 1-[2-(4-fluorophenyl)-6-(trifluoromethyl)pyrazolo[1,5-a]pyridin-3-yl]-2-propyn-1-one (3.44 g, 10.4 mmol) portionwise. The reaction mixture was stirred at 0° C. for 30 minutes, followed by room temperature for 15 hours. The reaction mixture was diluted with water (400 mL). The solid precipitate was collected on a filter to provide N-cyclopentyl-4-[2-(4-fluorophenyl)-6-(trifluoromethyl)-pyrazolo[1,5-a]pyridin-3-yl]-2-pyrimidinamine (4.48 g, 98%) as an orange solid. ¹H NMR (400 MHz, CDCl₃) δ 8.83 (s, 1H), 8.50 (d, 1H), 8.11 (d, 1H), 7.63 (m, 2H), 7.43 (d, 1H), 7.16 (t, 2H), 6.34 (d, 1H), 5.17 (d, 1H), 4.34 (m, 1H), 2.09 (m, 2H), 1.80-1.55 (m, 6H); MS m/z 442 (M+1); mp 155-156° C.

### e) N-Cyclopentyl-4-[2-(4-fluorophenyl)-6-(triethoxymethyl)pyrazolo[1,5-a]pyridin-3-yl]-2-pyrimidinamine

To a dry round bottom flask was added sodium metal (1.9 g, 83 mmol). Ethanol (110 mL) was added and allowed to react with sodium at room temperature until completely dissolved. N-Cyclopentyl-4-[2-(4-fluorophenyl)-6-(trifluoromethyl)-pyrazolo[1,5-a]pyridin-3-yl]-2-pyrimidinamine (4.48 g, 10.1 mmol) was added and the reaction mixture was stirred at 60° C. for 18 hours. The reaction mixture was cooled and concentrated in vacuo to approximately one-fourth of the original volume. The resulting mixture was diluted with water and extracted with ethyl acetate. The organic layer was washed with water and brine, then dried over magnesium sulfate. Filtration and concentration provided N-cyclopentyl-4-[2-(4-fluorophenyl)-6-(triethoxymethyl)pyrazolo[1,5-a]pyridin-3-yl]-2-pyrimidinamine (4.86 g, 92%) as an off-white solid. R_{f} 0.15 (4:1 hexanes:ethyl acetate); ¹H NMR (300 MHz, CDCl₃) δ 8.81 (s, 1H), 8.39 (d, 1H), 8.06 (d, 1H), 7.62 (m, 2H), 7.47 (d, 1H), 7.14 (t, 2H), 6.32 (d, 1H), 5.12 (d, 1H), 4.35 (m, 1H), 3.43 (q, 6H), 2.08 (m, 2H), 1.80-1.51 (m, 6H), 1.21 (t, 9H); MS m/z 520 (M+1).

### f) Ethyl 3-[2-(cyclopentylamino)-4-pyrimidinyl]-2-(4-fluorophenyl)pyrazolo[1,5-a]pyridine-6-carboxylate

To a solution of N-cyclopentyl-4-[2-(4-fluorophenyl)-6-(triethoxymethyl)pyrazolo-[1,5-a]pyridin-3-yl]-2-pyrimidinamine (1.0 g, 1.9 mmol) in acetone (40 mL) and water (10 mL) was added p-toluenesulfonic acid monohydrate (915 mg, 4.81 mmol). The reaction mixture was stirred at room temperature for 2 hours. The pH of the reaction mixture was adjusted to slightly basic using saturated aqueous sodium bicarbonate solution. The reaction mixture was concentrated in vacuo to one third of the original volume, then diluted with water. The precipitate was collected on a filter to provide ethyl 3-[2-(cyclopentylamino)-4-pyrimidinyl]-2-(4-fluorophenyl)pyrazolo[1,5-a]pyridine-6-carboxylate (722 mg, 85%) as an orange solid. R_{f} 0.15 (4:1 hexanes:ethyl acetate); ¹H NMR (300 MHz, CDCl₃) δ 9.22 (s, 1H), 8.38 (d, 1H), 8.08 (br, 1H), 7.85 (d, 1H), 7.64 (m, 2H), 7.16 (t, 2H), 6.34 (s, 1H), 5.26 (br, 1H), 4.44 (q, 2H), 4.35 (br, 1H), 2.08 (m, 2H), 1.80-1.52 (m, 6H), 1.43 (t, 3H); MS m/z 446 (M+1).

g) To a cold (−78° C.) solution of ethyl 3-[2-(cyclopentylamino)-4-pyrimidinyl]-2-(4-fluorophenyl)pyrazolo[1,5-a]pyridine-6-carboxylate (722 mg, 1.62 mmol) in dichloromethane (14 mL) was added diisobutylaluminum hydride (6.5 mL, 1.0 M in hexanes, 6.5 mmol). The resultant solution was stirred at −78° C. for 1.5 hours. The reaction mixture was poured into saturated aqueous solution Rochelle's salt and stirred at room temperature for 2 hours. The resultant mixture was extracted with ethyl acetate. The organic layer was washed with water and brine, then dried over magnesium sulfate. Filtration and concentration followed by flash chromatography (4:1 dichloromethane:acetone) provided [3-[2-(cyclopentylamino)-4-pyrimidinyl]-2-(4-fluorophenyl)pyrazolo[1,5-a]pyridin-6-yl]methanol (261 mg, 40%) as a white solid. R_{f} 0.41 (4:1 dichloromethane:acetone); ¹H NMR (300 MHz, CDCl₃) δ 8.37 (s, 1H), 8.28 (d, 1H), 8.00 (d, 1H), 7.56 (m, 2H), 7.23 (d, 1H), 7.10 (t, 2H), 6.26 (d, 1H), 5.24 (d, 1H), 4.65 (s, 2H), 4.29 (m, 1H), 3.73 (br, 1H), 2.03 (m, 2H), 1.76-1.45 (m, 6H); MS m/z 404 (M+1).

### EXAMPLE 34

### N-Cyclopentyl-4-[2-(4-fluorophenyl)-6-methylpyrazolo[1,5-a]pyridin-3-yl]-2-pyrimidinamine

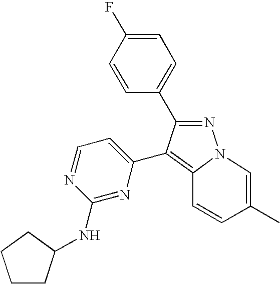

### a) 4-[6-(Bromomethyl)-2-(4-fluorophenyl)pyrazolo[1,5-a]pyridin-3-yl]-N-cyclopentyl-2-pyrimidinamine

To a solution of [3-[2-(cyclopentylamino)-4-pyrimidinyl]-2-(4-fluorophenyl)pyrazolo-[1,5-a]pyridin-6-yl]methanol (65 mg, 0.16 mmol) in chloroform (1 mL) was added phosphorus tribromide (6 μL, 0.06 mmol). The reaction mixture was stirred at room temperature for 2 hours, then quenched with saturated aqueous sodium bicarbonate solution. The resultant mixture was extracted with dichloromethane. The organic layer was washed with water and brine, then dried over sodium sulfate. Filtration and concentration followed by flash chromatography (19:1 dichloromethane:acetone) provided 4-[6-(bromomethyl)-2-(4-fluorophenyl)pyrazolo-[1,5-a]pyridin-3-yl]-N-cyclopentyl-2-pyrimidinamine (32 mg, 43%) as a yellow oil. R_{f} 0.68 (9:1 dichloromethane:acetone); ¹H NMR (400 MHz, CDCl₃) δ 8.49 (s, 1H), 8.39 (d, 1H), 8.05 (d, 1H), 7.60 (m, 2H), 7.34 (d, 1H), 7.13 (t, 2H), 6.30 (d, 1H), 5.21 (d, 1H), 4.53 (s, 2H), 4.33 (m, 1H), 2.07 (m, 2H), 1.78-1.51 (m, 6H); MS m/z 467 (M+1).

### b) N-Cyclopentyl-4-[2-(4-fluorophenyl)-6-methylpyrazolo[1,5-a]pyridin-3-yl]-2-pyrimidinamine

To a solution of 4-[6-(bromomethyl)-2-(4-fluorophenyl)pyrazolo[1,5-a]pyridin-3-yl]-N-cyclopentyl-2-pyrimidinamine (40 mg, 0.086 mmol) in toluene (5 mL) was added tributyltin hydride (35 μL, 0.13 mmol) and 2,2′-azobisisobutyronitrile (2 mg, 0.009 mmol). The reaction mixture was heated at 95° C. for 3 hours. After cooling the reaction mixture room temperature, Celite was added and the resultant mixture was concentrated in vacuo. Flash chromatography (19:1 dichloromethane:acetone) provided a crude material which was triturated with ether to provide N-cyclopentyl-4-[2-(4-fluorophenyl)-6-methylpyrazolo[1,5-a]pyridin-3-yl]-2-pyrimidinamine (4 mg, 12%) as a pale yellow solid. R_{f} 0.63 (9:1 dichloromethane:acetone); ¹H NMR (300 MHz, CDCl₃) δ 8.37-8.28 (m, 2H), 8.00 (br, 1H), 7.60 (m, 2H), 7.23-7.11 (m, 3H), 6.32 (d, 1H), 5.37 (br, 1H), 4.36 (m, 1H), 2.40 (s, 3H), 2.07 (m, 2H), 1.82-1.51 (m, 6H); MS m/z 388 (M+1).

### EXAMPLE 35

### N-Cyclopentyl-4-[6-[(cyclopentylamino)methyl]-2-(4-fluorophenyl)-pyrazolo[1,5-a]pyridin-3-yl]-2-pyrimidinamine

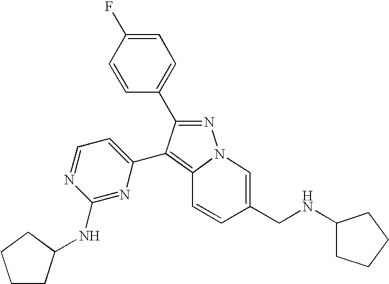

To a suspension of 4-[6-(bromomethyl)-2-(4-fluorophenyl)pyrazolo[1,5-a]pyridin-3-yl]-N-cyclopentyl-2-pyrimidinamine (30 mg, 0.064 mmol) in tetrahydrofuran (2 mL) was added cyclopentylamine (730 μL, 7.4 mmol). The reaction mixture was stirred at room temperature for 3 hours, then diluted with ethyl acetate. The organic layer was washed with saturated aqueous sodium bicarbonate solution, water, and brine. The organic layer was dried over magnesium sulfate. Filtration and concentration followed by flash chromatography (39:1 dichloromethane:methanol to 35:5 dichloromethane:methanol) provided N-cyclopentyl-4-[6-[(cyclopentylamino)methyl]-2-(4-fluorophenyl)pyrazolo[1,5-a]pyridin-3-yl]-2-pyrimidinamine (6 mg, 20%) as a light yellow solid. R_{f} 0.56 (35:5 dichloromethane:methanol); ¹H NMR (400 MHz, CDCl₃) δ 8.46 (s, 1H), 8.36 (d, 1H), 8.04 (d, 1H), 7.60 (m, 2H), 7.37 (d, 1H), 7.12 (t, 2H), 6.30 (d, 1H), 5.10 (d, 1H), 4.33 (m, 1H), 3.84 (s, 2H), 3.14 (m, 1H), 2.07 (m, 2H), 1.85 (m, 2H), 1.79-1.38 (m, 12H); MS m/z 471 (M+1).

### EXAMPLE 36

### 4-[5-Chloro-2-(4-fluorophenyl)pyrazolo[1,5-a]pyridin-3-yl]-N-cyclopentyl-2-pyrimidinamine

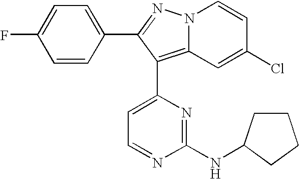

### a) 2-(4-Chloro-2-pyridinyl)-1-(4-fluorophenyl)ethanone

To a cold (0° C.) solution of 4-chloro-2-picoline (5.0 g, 39 mmol) and ethyl 4-fluorobenzoate (6.6 g, 39 mmol) in tetrahydrofuran (100 mL) was added lithium bis(trimethylsilyl)amide (80 mL, 1.0 M in tetrahydrofuran, 80 mmol) dropwise via a pressure equalizing funnel over 30 minutes. Upon complete addition, the cold bath was removed and the resulting solution was stirred at room temperature for 15 hours. The reaction mixture was concentrated under reduced pressure and methanol was added to the reaction, resulting in the formation of a white precipitate. The precipitate was collected by filtration and dried to give 2-(4-chloro-2-pyridinyl)-1-(4-fluorophenyl)ethanone (9.6 g, 9901%) as a white solid. ¹H-NMR (DMSO-d₆): δ 7.90 (m, 3H), 7.11 (t, 2H), 6.56 (s, 1H), 5.67 (s, 1H), 4.14 (m, 2H); ¹⁹F-NMR (DMSO-d₆): 6-115.67; MS m/z 250 (M+1).

### b) 2-(4-Chloro-2-pyridinyl)-1-(4-fluorophenyl)ethanone oxime

To a solution of 2-(4-chloro-2-pyridinyl)-1-(4-fluorophenyl)ethanone (9.6 g, 38 mmol) in methanol (200 mL) was added hydroxylamine hydrochloride (13.5 g, 190 mmol) followed by the addition of a sodium hydroxide solution (7.8 g, 190 mmol in 50 mL of water). The resulting suspension was heated at reflux for 2 hours and then allowed to cool to room temperature. The mixture was concentrated and water was added to the resulting slurry. A white precipitate formed, which was collected by filtration, washed with water and dried (magnesium sulfate) to give 2-(4-chloro-2-pyridinyl)-1-(4-fluorophenyl)ethanone oxime (8.45 g, 84%) as a white solid. ¹H-NMR (DMSO-d₆): δ 11.56 (s, 1H), 8.44 (d, 1H), 7.80 (m, 2H), 7.40 (m, 2H), 7.22 (m, 2H), 4.29 (s, 2H); ¹⁹F-NMR (DMSO-d₆): δ −113.44; MS m/z 265 (M+1).

### c) 5-Chloro-2-(4-fluorophenyl)pyrazolo[1,5-a]pyridine

To a solution of 2-(4-chloro-2-pyridinyl)-1-(4-fluorophenyl)ethanone oxime (8.0 g, 30 mmol) in 1,2-dimethoxyethane (50 mL) at 0° C. was added trifluoroacetic anhydride (6.3 g, 30 mmol), keeping the temperature below 10° C. during the addition. After the addition was complete, the reaction was warmed to room temperature. The solution was then cooled to 4° C. and a solution of triethylamine (8.4 mL, 60 mmol) in 1,2-dimethoxyethane (20 mL) was added over a period of 0.5 hours. The mixture was allowed to warm to room temperature and was stirred for 1.5 hours. To this mixture was added iron(II) chloride (40 mg) and the reaction was heated at 75° C. for 15 hours. The reaction mixture was poured into water (300 mL). The resulting suspension was extracted with ethyl acetate. The combined organics were dried (magnesium sulfate), filtered and concentrated to a solid residue. This residue was purified by flash chromatography (1:1 ethyl acetate-hexane) to give 5-chloro-2-(4-fluorophenyl)pyrazolo[1,5-a]pyridine (4.2 g, 57%) as a white solid. ¹H-NMR (CDCl₃): δ 8.36 (d, 1H), 7.93 (q, 2H), 7.49 (d, 1H), 7.15 (t, 2H), 6.70 (dd, 1H), 6.69 (s, 1H); ¹⁹F-NMR (CDCl₃): 8-113.30; MS m/z 247 (M+1).

### d) 5-Chloro-2-(4-fluorophenyl)pyrazolo[1,5-a]pyridine-3-carbaldehyde

Phosphorous oxychloride (0.6 mL, 6.4 mmol) was added to N,N-dimethylformamide (10 mL) and the resulting mixture stirred at room temperature for 10 minutes. 5-Chloro-2-(4-fluorophenyl)pyrazolo[1,5-a]pyridine (1.0 g, 4.1 mmol) was added and the reaction mixture was stirred at room temperature for 12 hours. The reaction mixture was poured into ice-water and neutralized to pH 7 with aquous ammonium hydroxide. The resulting slurry was extracted with dichloromethane (3×40 mL). The combined organics were washed with brine, dried (magnesium sulfate), filtered and concentrated to give, after recrystallization from acetonitrile, 5-chloro-2-(4-fluorophenyl)pyrazolo [1,5-a]pyridine-3-carbaldehyde (0.95 g, 85%) as a white solid. ¹H-NMR (CDCl₃): δ10.07 (s, 1H), 8.49 (d, 1H), 8.44 (d, 1H), 7.78 (q, 2H), 7.22 (t, 2H), 7.07 (dd, 1H); MS m/z 275 (M+1).

### e) 1-[5-Chloro-2-(4-fluorophenyl)pyrazolo[1,5-a]pyridin-3-yl]-2-butyn-1-one

To a solution of 5-chloro-2-(4-fluorophenyl)pyrazolo[1,5-a]pyridine-3-carbaldehyde (0.93 g, 3.4 mmol) in tetrahydrofuran (20 mL) at −78° C. was added ethynylmagnesium bromide (16 mL, 0.5 M in tetrahydrofuran, 8.0 mmol). The mixture was allowed to warm to room temperature and stirred for 1 hour. Water was added to the reaction and the resulting mixture was extracted with ethyl acetate. The ethyl acetate phase was dried (magnesium sulfate), filtered and concentrated to a solid residue. This residue was dissolved in dichloromethane (50 mL) and manganese dioxide (5 g) was added. This slurry was stirred at room temperature for 2 hours. The manganese dioxide was removed by filtration and the filtrate was concentrated to a solid. This solid was purified by flash chromatography (dichloromethane) to give 1-[5-chloro-2-(4-fluorophenyl)pyrazolo[1,5-a]pyridin-3-yl]-2-butyn-1-one (0.63 g, 62% for two steps) as a white solid. ¹H-NMR (CDCl₃): δ 8.52 (d, 1H), 8.47 (d, 1H), 7.69 (q, 2H), 7.18 (t, 2H), 7.07 (dd, 1H), 3.00 (s, 1H); ¹⁹F-NMR (CDCl₃): δ −111.69; MS m/z 299 (M+1).

### f) 4-[5-Chloro-2-(4-fluorophenyl)pyrazolo[1,5-a]pyridin-3-yl]-N-cyclopentyl-2-pyrimidinamine

To a solution of 1-[5-chloro-2-(4-fluorophenyl)pyrazolo[1,5-a]pyridin-3-yl]-2-butyn-1-one (0.61 g, 2.0 mmol) in N,N-dimethylformamide was added cyclopentyl guanidine hydrochloride (0.67 g, 4.1 mmol) followed by anhydrous potassium carbonate (0.57 g, 4.1 mmol). The resulting mixture was heated at 80° C. for 12 hours. Upon cooling to room temperature, water was added. The mixture was extracted with ethyl acetate. The ethyl acetate phase was washed with brine, dried (magnesium sulfate), filtered and concentrated in vacuo. The resulting residue was purified by flash chromatography (1:1 ethyl acetate-hexane) to give, after recrystallization from acetonitrile, 4-[5-chloro-2-(4-fluorophenyl)pyrazolo[1,5-a]pyridin-3-yl]-N-cyclopentyl-2-pyrimidinamine (0.6 g, 74%) as a white solid. ¹H-NMR (CDCl₃): 68.54 (broad s, 1H), 8.40 (d, 1H), 8.04 (d, 1H), 7.60 (q, 2H), 7.16 (t, 2H), 6.88 (dd, 1H), 6.28 (d, 1H), 5.22 (d, 1H), 4.40 (m, 1H), 1.4-2.2 (m, 8H); ¹⁹F-NMR (CDCl₃): 8-112.5; MS m/z 408 (M+1).

### EXAMPLE 37

### N-Cyclopentyl-4-[2-(4-fluorophenyl)-5-(1-pyrrolidinyl)pyrazolo[1,5-a]pyridin-3-yl]-2-pyrimidinamine

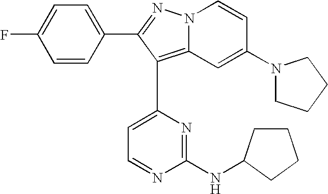

To a solution of 4-[5-chloro-2-(4-fluorophenyl)pyrazolo[1,5-a]pyridin-3-yl]-N-cyclopentyl-2-pyrimidinamine (0.1 g, 0.25 mmol) in pyrrolidine (5 mL) was added rac-2,2′-bis(diphenylphosphino)-1,1′-binaphthyl (46 mg, 0.08 mmol), cesium carbonate (120 mg, 0.38 mmol) and palladium (II) acetate (11 mg, 0.05 mmol). The resulting mixture was stirred at 80° C. for 24 hours, at which time the reaction was judged complete by thin layer chromatography. The solution was cooled to room temperature and ethyl acetate and water were added to the reaction mixture. The phases were separated, and the aqueous phase again extracted with ethyl acetate. The combined organic phases were dried (magnesium sulfate), filtered and concentrated. The resulting residue was purified by flash chromatography (1:1 hexanes-ethyl acetate) to give N-cyclopentyl-4-[2-(4-fluorophenyl)-5-(1-pyrrolidinyl) pyrazolo[1,5-a]pyridin-3-yl]-2-pyrimidinamine (60 mg, 54%) as a solid. ¹H-NMR (CDCl₃): 68.23 (d, 1H), 7.92 (d, 1H), 7.58 (m, 2H), 7.35 (s, 1H), 7.12 (t, 2H), 6.43 (dd, 1H), 6.2 (d, 1H), 5.00 (d, 1H), 4.46 (m, 1H), 3.42 (m, 4H), 2.06-1.4 (m, 12H); ¹⁹F-NMR (CDCl₃): δ −113.69; MS m/z 443 (M+1).

### EXAMPLE 38

### 4-[5-Chloro-2-(4-methoxyphenyl)pyrazolo[1,5-a]pyridin-3-yl]-N-cyclopentyl-2-pyrimidinamine

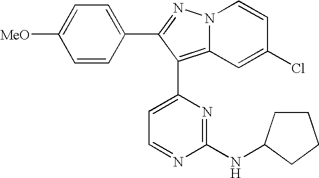

### a) 2-(4-Chloro-2-pyridinyl)-1-(4-methoxyphenyl)ethanone

To a cold (0° C.) solution of 4-chloro-2-picoline (10 g, 78.4 mmol) and ethyl 4-methoxybenzoate (14.1 g, 78.4 mmol) in tetrahydrofuran (100 mL) was added lithium bis(trimethylsilyl)amide (157 ml 1.0 M in tetrahydrofuran, 157 mmol) dropwise via a pressure equalizing funnel over half an hour. Upon complete addition, the ice bath was removed and the resulting solution was heated at 45° C. for 15 hours. The mixture was cooled to room temperature, and the solution was concentrated. Methanol was added to quench the reaction, resulting in the formation of a yellow precipitate. The precipitate was collected by filtration and dried to give the product as a mixture of enol and ketone tautomers. MS m/262 (M+1).

### b) 2-(4-Chloro-2-pyridinyl)-1-(4-methoxyphenyl)ethanone Oxime

To a solution of 2-(4-chloro-2-pyridinyl)-1-(4-methoxyphenyl)ethanone in methanol (200 mL) was added hydroxylamine hydrochloride (27.2 g, 392 mmol) followed by the addition of a sodium hydroxide solution (15.7 g, 392 mmol in 50 mL of water). The resulting suspension was heated at reflux for 1 hour and then allowed to cool to room temperature. The mixture was concentrated and water was added to the resulting slurry. A white precipitate formed, which was collected by filtration, washed with water and dried to give 2-(4-chloro-2-pyridinyl)-1-(4-methoxyphenyl)ethanone oxime (11.8 g) as a white solid. ¹H NMR (CDCl₃): δ 8.47 (d, 1H), 7.72 (d, 2H), 7.36 (d, 1H), 7.19 (dd, 1H), 6.91 (d, 2H), 4.43 (s, 2H), 3.84 (s, 3H); MS m/z 277 (M+1).

### c) 5-Chloro-2-(4-methoxyphenyl)pyrazolo[1,5-a]pyridine

To a solution of 2-(4-chloro-2-pyridinyl)-1-(4-methoxyphenyl)ethanone oxime (11.8 g, 42.6 mmol) in 1,2-dimethoxyethane (200 mL) at 0° C. was added trifluoroacetic anhydride (6.3 mL, 44.8 mmol), keeping the temperature below 10° C. during the addition. After the addition was complete, the reaction was warmed to 15° C. The solution was then cooled to 4° C. and a solution of triethylamine (12.5 mL 89.5 mmol) in 1,2-dimethoxyethane (15 mL) was added over a period of 0.5 hours. The mixture was allowed to warm to room temperature and was stirred at room temperature for 5 hours. To this mixture was added iron(II)chloride (0.11 g, 0.85 mmol) and the reaction was heated at 75° C. for 15 hours. The reaction mixture was poured into water (300 mL). The resulting suspension was extracted with ethyl acetate. The organic phase was dried (magnesium sulfate), filtered and concentrated to a solid. This solid was recrystallized from methanol to give 5-chloro-2-(4-methoxyphenyl)pyrazolo[1,5-a]pyridine (6.64 g, 600/%) as white needles. ¹H NMR (CDCl₃): δ 8.35 (d, 1H), 7.86 (d, 2H), 7.46 (d, 1H), 6.97 (d, 2H), 6.67 (d, 1H), 6.65 (s, 1H), 3.85 (s, 3H); MS m/z 259 (M+1).

### d) 5-Chloro-2-(4-methoxyphenyl)pyrazolo[1,5-a]pyridine-3-carbaldehyde

To N,N-dimethylformamide (20 mL) at 0° C. was added phosphorous oxychloride (0.54 mL, 7.8 mmol). After the addition was complete, the mixture was warmed to room temperature and stirred for 1 hour. To this was added 5-chloro-2-(4-methoxyphenyl)pyrazolo[1,5-a]pyridine (1.0 g, 3.86 mmol) and the resultant solution was stirred 2 hours. Water was added, followed by dichloromethane. The aqueous layer was extracted with dichloromethane. The combined organics were washed with brine, dried over magnesium sulfate, filtered and concentrated. A white crystalline compound, 5-chloro-2-(4-methoxyphenyl)pyrazolo[1,5-a]pyridine-3-carbaldehyde (0.9 g, 81%), was obtained. ¹H NMR (CDCl₃): δ 10.12 (s, 1H), 8.52 (d, 1H), 8.47 (d, 1H), 7.76 (d, 2H), 7.11-7.06 (m, 3H), 3.93(s, 3H); MS m/z 287 (M+1).

### e) 1-[5-Chloro-2-(4-methoxyphenyl)pyrazolo[1,5-a]pyridin-3-yl]-2-propyn-1-ol

To a cold (−78° C.) suspension of 5-chloro-2-(4-methoxyphenyl)pyrazolo[1,5-a]pyridine-3-carbaldehyde (0.90 g, 3.14 mmol) in tetrahydrofuran (50 mL) was added ethynylmagnesium bromide (7.5 mL, 0.5 M in tetrahydrofuran, 3.77 mmol) dropwise. The reaction mixture was stirred at −78° C. for 1 hour, then at room temperature for 4 hours. The resultant solution was poured into saturated aqueous sodium bicarbonate and extracted with ethyl acetate. The organic layer was washed with water and brine and the combined organics were dried over magnesium sulfate. Filtration and concentration provided 1-[5-chloro-2-(4-methoxyphenyl)pyrazolo[1,5-a]pyridin-3-yl]-2-propyn-1-ol (1.05 g, 100%) as a white solid. ¹H NMR (CDCl₃) δ 8.40 (d, 1H), 8.05 (s, 1H), 7.72 (d, 2H), 7.05 (d, 2H), 6.80 (dd, 1H), 5.78 (s, 1H), 3.91 (s, 3H), 2.74 (s, 1H), 2.53 (s, 1H); MS m/z 313 (M+1).

### f) 1-[5-Chloro-2-(4-methoxyphenyl)pyrazolo[1,5-a]pyridin-3-yl]-2-propyn-1-one

To a solution of 1-[5-chloro-2-(4-methoxyphenyl)pyrazolo[1,5-a]pyridin-3-yl]-2-propyn-1-ol (1.05 g, 3.14 mmol) in chloroform (100 mL) was added manganese dioxide (6.82 g, 78.5 mmol). The reaction mixture was stirred at, room temperature for 3.5 hours. The suspension was filtered through a pad of Celite and the filtrate was concentrated to give 1-[5-chloro-2-(4-methoxyphenyl)pyrazolo[1,5-a]pyridin-3-yl]-2-propyn-1-one (0.99 g, 100%) as a pale yellow solid. ¹H NMR (CDCl₃) 68.50 (d, 1H), 8.46 (d, 1H), 7.64 (d, 2H), 7.04 (dd, 1H), 6.98 (d, 2H), 3.87 (s, 3H), 2.99 (s, 1H); MS m/z 295 (M+1).

### g) 4-[5-Chloro-2-(4-methoxyphenyl)pyrazolo[1,5-a]pyridin-3-yl]-N-cyclopentyl-2-pyrimidinamine

Sodium ethylate (0.7 mL (2.09 mmol), 21% in ethanol) and cyclopentyl guanidine hydrochloride (0.47 g, 2.88 mmol) were added sequentially to ethanol (30 mL). The resulting solution was stirred at room temperature for 30 minutes. 1-[5-chloro-2-(4-methoxyphenyl)pyrazolo[1,5-a]pyridin-3-yl]-2-propyn-1-one (0.5 g, 1.61 mmol) was added, and the suspension was stirred at room temperature for 2 days. The reaction was quenched by the addition of water. The aqueous phase was extracted by ethyl acetate. The organics were combined, washed with brine and dried over magnesium sulfate. Filtration and concentration gave a solid. This solid was recrystallized from methanol to give 4-[5-chloro-2-(4-methoxyphenyl)pyrazolo[1,5-a]pyridin-3-yl]-N-cyclopentyl-2-pyrimidinamine (0.45 g, 66%) as a pale yellow solid. ¹H NMR (CDCl₃) δ 8.59 (broad s, 1H), 8.42 (d, 1H), 8.05 (d, 1H), 7.59 (d, 2H), 7.03 (d, 2H), 6.91 (dd, 1H), 6.39 (d, 1H), 5.34 (broad s, 1H), 4.42 (m, 1H), 3.92 (s, 3H), 2.17 (m, 2H), 1.86-1.60 (m, 6H); MS m/z 420 (M+1).

### EXAMPLES 39-49

Using the techniques described above for Examples 1-38, the following additional compounds are prepared.

| Example No. | Compound Name | Structure |
|---|---|---|
| 39 | 1-[3-({4-[2-(4-fluorophenyl)- pyrazolo[1,5-a]pyridin-3-yl]-2- pyridinyl}amino)propyl]-2- pyrrolidinone | 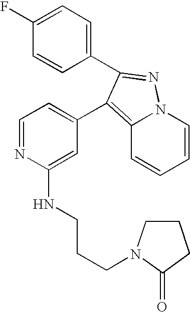 |
| 40 | 6-Fluoro-4-[2-(4-fluorophenyl)- pyrazolo[1,5-a]pyridin-3-yl]-N- methyl-2-pyridinamine | 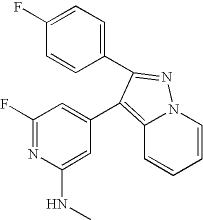 |
| 41 | 4-[4-Fluoro-2-(4-fluorophenyl)- pyrazolo[1,5-a]pyridin-3-yl]-N,N- dimethyl-2-pyridinamine | 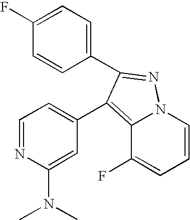 |
| 42 | N-Allyl-4-[2-(4-fluorophenyl)pyrazolo- [1,5-a]pyridin-3-yl]-2-pyridinamine | 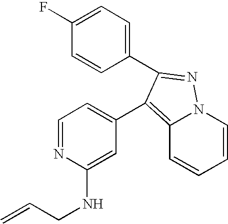 |
| 43 | 5-[6-Chloro-2-(4-fluorophenyl)- pyrazolo[1,5-a]pyridin-3-yl]-N- cyclopropyl-2-pyridinamine | 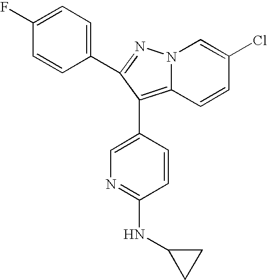 |
| 44 | 3-({5-Bromo-4-[2-(4-fluorophenyl)- pyrazolo[1,5-a]pyridin-3-yl]-2- pyridinyl}amino)-1-propanol | 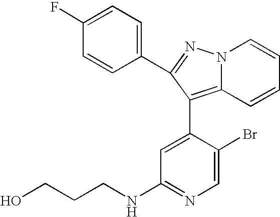 |
| 45 | Methyl 3-(2-{[3-(acetyloxy)propyl]- amino}-4-pyridinyl)-2-(4- fluorophenyl)pyrazolo[1,5-a]pyridine-6- carboxylate | 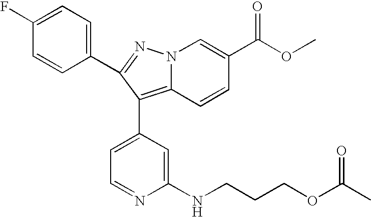 |
| 46 | 3-[2-(Cyclopropylamino)-4- pyrimidinyl]-2-(4- fluorophenyl)pyrazolo[1,5-a]pyridine-6- carboxylic acid | 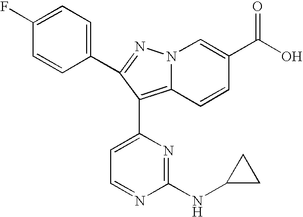 |
| 47 | 3-[2-(Cyclopropylamino)-4- pyrimidinyl]-2-(4-fluorophenyl)-N,N- dimethylpyrazolo[1,5-a]pyridine-6- carboxamide | 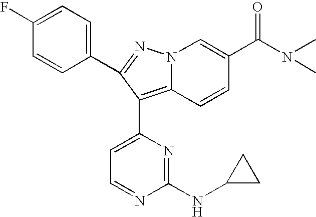 |
| 48 | N-Cyclopropyl-3-[2- (cyclopropylamino)-4-pyrimidinyl]-2- (4-fluorophenyl)pyrazolo[1,5- a]pyridine-6-carboxamide | 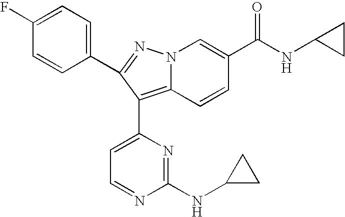 |
| 49 | N-Cyclopentyl-4-[2-(4- fluorophenyl)pyrazolo[1,5-a]pyridin-3- yl]-6-phenyl-2-pyrimidinamine | 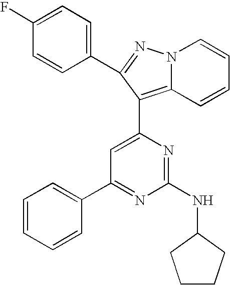 |

### EXAMPLE 50

### 2-(4-Fluorophenyl)-3-(4-pyrimidinyl)-pyrazolo[1,5-a]pyridine

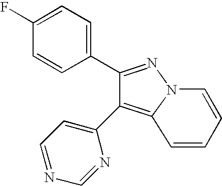

### a) 1-(4-Fluorophenyl)-2-(4-pyrimidinyl)-ethanone

To a stirred solution of 4-methylpyrimidine (20.64 g, 0.22 mol) and ethyl 4-fluorobenzoate (36.9 g, 0.22 mol) in dry tetrahydrofuran (100 mL) at 0° C. under nitrogen was added lithium bis(trimethylsilyl)amide (1M in tetrahydrofuran, 440 mL, 0.44 mol) over a 2 hour period. A white precipitate deposited during the addition and this suspension was stirred at room temperature overnight. The reaction was diluted with 100 mL of water and filtered. The filtrate was washed with water three times and dried. The solution was diluted with ethyl acetate (100 mL) and the organic phase separated. The aqueous phase was further extracted with ethyl acetate (100 mL). Organic phases were dried over magnesium sulfate and concentrated and combined with the filtrate to give a combined yield of 47 g (98%) of product. ¹H NMR (CDCl₃) exists as a 2:1 mixture of enol:keto tautomers: δ enol form: 5.95 (s, 1H), 6.92 (dd, J=1.2, 5.7 Hz, 1H), 7.06-7.14 (m, 2H), 7.83 (dd, J=5.4, 8.7 Hz, 2H), 8.40 (d, J=5.7 Hz, 1H), 8.8 (s, 1H); keto form: 4.42 (s, 2H), 7.12-7.18 (m, 2H), 7.34 (d, J=4.2 Hz, 1H), 8.06 (dd, J=5.3, 8.8 Hz, 2H), 8.67 (d, J=5.1 Hz, 1H), 9.16 (s, 1H); APESI−MS m/215 (M−1)⁻.

b) A solution of 1-(4-fluorophenyl)-2-(4-pyrimidinyl)-ethanone (21.6 g, 0.1 mol), 1-aminopyridium iodide (22.2 g, 0.1 mol) and potassium carbonate (41.4 g, 0.3 mol) in a mixture of water (300 mL) and isopropanol (300 mL) was heated and stirred at 100° C. for 16 hours. The isopropanol was removed under vacuum and the resulting aqueous phase extracted with dichloromethane (5×200 mL). The dichloromethane extracts were combined and the solvent evaporated under reduced pressure to leave a red solid which was purified by silica gel chromatography eluting with a hexane/ethyl acetate to give the title compound as a yellow solid, 9.16 g (32%). ¹H NMR (DMSO-d₆): δ 7.07 (d, J=5.4 Hz, 1H), 7.14 (t, J=6.8 Hz, 1H), 7.32 (t, J=8.7 Hz, 2H), 7.53 (t, J=7.8 Hz, 1H), 7.60 (dd, J=5.7, 8.7 Hz, 2H), 8.40 (d, J=8.9 Hz, 1H), 8.54 (d, J=5.3 Hz, 1H), 8.83 (d, J=7.1 Hz, 1H), 9.16 (s, 1H), APESI+MS m/z 291 (M+1).

### EXAMPLE 51

### 2-(4-Fluorophenyl)-3-(4-(2-methylthio)pyrimidinyl)-pyrazolo[1,5-a]pyridine

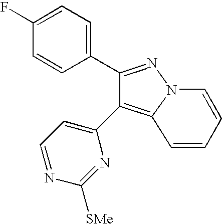

### a) 1-(4-Fluorophenyl)-2-(4-(2-methylthio) pyrimidinyl)ethanone

To a stirred solution of 2-methylthio-4-methylpyrimidine (66 g, 0.47 mol) and ethyl 4-fluorobenzoate (79 g, 0.47 mol) in dry tetrahydrofuran (400 mL) at 0° C. under nitrogen was added lithium bis(trimethylsilyl)amide (1N in tetrahydrofuran, 940 mL, 0.94 mol) over a 2 hour period. The solution was stirred at ice bath temperature for 18 hours. The solution was poured into 2 L of ice cold 0.5 N hydrochloric acid. A precipitate formed which was filtered off and air dried. Second and third crops of solids were obtained as the precipitate was washed with water. The combined precipitates were recrystalized from acetone and water to give product as a yellow solid: 117 g (95%). ¹H NMR (CDCl₃): 8 (all in enol form): 3.0 (s, 3H), 6.29 (s, 1H), 7.01 (d, J=5.7 Hz, 1H), 7.48 (t, J=8.7 Hz, 2H), 8.20 (dd, J=5.4, 8.8 Hz, 2H), 8.68 (d, J=5.7 Hz, 1H); APESI−MS m/z 261 (M−1)⁻.

b) A solution of 1-(4-fluorophenyl)-2-(4-(2-methylthio)pyrimidinyl)ethanone (13.0 g, 50 mmol) in isopropanol (300 mL) was warmed to reflux. A solution of 1-aminopyridinium iodide (14 g, 63 mmol) in water (300 mL) was treated with 2N sodium hydroxide (31.5 mL). This solution was added to the ketone over a period of two hours while the mixture was heated at reflux. After an additional seven hours, the isopropanol was partially evaporated under reduced pressure and the resulting solution was extracted with dichloromethane (2×300 mL). The dichloromethane extracts were combined, dried (magnesium sulfate), filtered and the solvent evaporated under reduced pressure to leave a red solid which was purified by silica gel chromatography with dichloromethane to give the title compound as a yellow solid, 4.5 g (26%). ¹H NMR (DMSO-d₆): δ 2.5 (s, 3H), 6.80 (d, J=5.3 Hz, 1H), 7.18 (t, J=6.9 Hz, 1H), 7.36 (t, J=8.8 Hz, 2H), 7.59 (t, J=7.9 Hz, 1H), 7.60 (dd, J=5.7, 8.7 Hz, 2H), 8.38 (d, J=9.1 Hz, 1H), 8.40 (d, J=5.3 Hz, 1H), 8.88 (d, J=7.0 Hz, 1H), APESI+MS m/z 337 (M+1).

### EXAMPLE 52

### 2-(4-Fluorophenyl)-3-(4-(2-methylsulfinyl)pyrimidinyl)-pyrazolo[1,5-a]pyridine

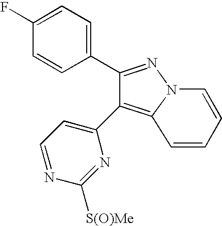

To a stirred solution of 2-(4-fluorophenyl)-3-(4-(2-methylthio)pyrimidinyl)-pyrazolo[1,5-a]pyridine (0.285 g, 0.85 mmol) in dichloromethane (10 mL) was added, dropwise, a solution of (0.257 g, 0.85-1.23 mmol) of 57-86% m-chloroperoxybenzoic acid in dichloromethane (5 mL). After 10 minutes, the solution was quenched by the addition of aqueous potassium carbonate (20 mL), and the organic phase was separated. The aqueous phase was further extracted with dichloromethane (2×20 mL) and the dichloromethane phases dried over magnesium sulfate filtered and concentrated to give a crude white solid. Chromatography on silica gel eluting with a hexane/Ethyl acetate gradient (0-100% Ethyl acetate) gave the title compound as a white solid, 0.213 g (60: ¹H NMR (CDCl₃): δ 3.05 (s, 3H), 7.07-7.11 (m, 2H), 7.25 (d, J=8.5 Hz, 2H), 7.55 (t, J=7.8 Hz, 1H), 7.64 (dd, J=5.5, 6.9 Hz, 2H), 8.52 (d, J=5.1 Hz, 1H), 8.59 (d, J=6.9 Hz, 1H), 8.84 (d, J=9.0 Hz, 1H); APESI+MS m/z 353 (M+1)⁻.

### EXAMPLE 53

### 2-(4-Fluorophenyl)-7-methyl-3-(4-pyrimidinyl)pyrazolo[1,5-a]pyridine

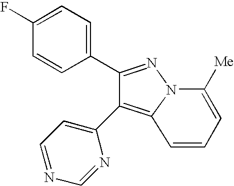

A solution of 2-(4-fluorophenyl)-3-(4-pyrimidinyl)-pyrazolo[1,5-a]pyridine (Example 50, 0.2 g, 0.69 mmol) in dry tetrahydrofuran (5 mL) was cooled to −78° C. under nitrogen and lithium diisopropylamide (0.45 mL of a 2M solution in heptane/tetrahydrofuran/ethylbenzene, 0.9 mmol) was added dropwise. The reaction mixture was stirred for about 10 minutes and methyl iodide (0.2 mL, 4 mmol) was added. The solution was allowed to warm to room temperature and stirred for a further 1.5 hours. The reaction mixture was diluted with diethyl ether (20 mL), water (20 mL) added, and the organic phase separated. The aqueous phase was further extracted with ether (20 mL) and the combined ether phases were dried over anhydrous magnesium sulfate, filtered and the solvents evaporated to give a yellow solid. Chromatography on silica gel eluting with 9:1 hexane/ethyl acetate gave the title compound, 0.080 g (380/%). ¹H NMR (DMSO-d₆): δ 2.72 (s, 3H), 7.05 (d, J=6.3 Hz, 2H), 7.32 (t, J=8.8 Hz, 2H), 7.46 (dd, J=7.0, 8.6 Hz, 1H), 7.61 (dd, J=5.5, 8.6 Hz, 2H), 8.32 (d, J=9.0 Hz, 1H), 8.52 (d, J=5.5 Hz, 1H), 9.15 (s, 1H); APESI+MS m/z 305 (M+1)⁻.

### EXAMPLE 54

### 2-(4-Fluorophenyl)-7-methylthio-3-(4-pyrimidinyl)pyrazolo[1,5-a]pyridine

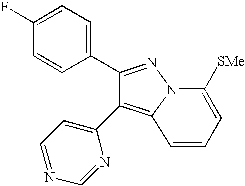

In a similar manner as described in Example 53, using dimethyl disulfide in place of methyl iodide, was obtained the title compound, (72%). ¹H NMR (DMSO-d₆): δ 2.46 (s, 3H), 7.01 (d, J=7.3 Hz, 1H), 7.06 (d, J=4.7 Hz, 1H), 7.33 (t, J=8.8 Hz, 2H), 7.53 (t, J=8.2 Hz, 1H), 7.61 (dd, J=5.5, 8.4 Hz, 2H), 8.22 (d, J=8.8 Hz, 1H), 8.53 (d, J=5.5 Hz, 1H), 9.15 (s, 1H); APCI+MS m/336 (M)⁻.

### EXAMPLE 55

### 2-(4-Fluorophenyl)-7-methylsulfinyl-3-(4-pyrimidinyl)pyrazolo[1,5-a]-pyridine

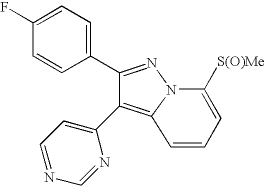

To a stirred solution of 2-(4-fluorophenyl)-7-methylthio-3-(4-pyrimidinyl)pyrazolo[1,5-a]pyridine (Example 54, 0.246 g, 0.73 mmol) in chloroform (20 mL) was added, dropwise, a solution of of m-chloroperbenzoic acid (57-860% o, 0.221 g, 0.73-1.1 mmol) in chloroform (10 mL). After 1 hour, the reaction was quenched by the addition of aqueous potassium carbonate (20 mL), and the organic phase was separated. The aqueous phase was further extracted with chloroform (2×20 mL) and the combined chloroform phases were dried over anhydrous magnesium sulfate. The drying agent was removed by filtration and the solvent was evaporated to give a light brown solid. Chromatography on silica gel eluting with a hexane/ethyl acetate gradient (0-30% ethyl acetate) gave the title compound as the major product, 0.170 g (66%). ¹H NMR (DMSO-d₆): δ 3.11 (s, 3H), 7.13 (d, J=5.4 Hz, 1H), 7.33 (t, J=8.8 Hz, 2H), 7.50 (d, J=7.0 Hz, 1H), 7.63 (dd, J=5.7, 8.6 Hz, 2H), 7.76 (dd, J=7.4, 8.1 Hz, 1H), 8.50 (d, J=8.8 Hz, 1H), 8.60 (d, J=5.5 Hz, 1H), 9.20 (s, 1H); APESI+MS m/z 353 (M+1)⁻.

### EXAMPLE 56

### 7-(2-Fluoroethoxy)-2-(4-fluorophenyl)-3-(4-(2-methylthio)pyrimidinyl)-pyrazolo[1,5-a]pyridine

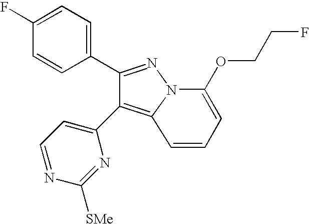

### a) 7-(2-Fluoroethoxy)-2-(4-fluorophenyl)-3-(4-(2-methylthio)pyrimidinyl)-pyrazolo[1,5-a]pyridine

To a stirred solution of 2-fluoroethanol (0.128 g, 2 mmol) in tetrahydrofuran (5 mL), under nitrogen, was added potassium tert-butoxide (1M in tert-BuOH. 2.0 mL, 2 mmol) and the resulting solution stirred for 5 minutes. A solution of 7-chloro-2-(4-fluorophenyl)-3-(4-(2-methylthio)pyrimidinyl)pyrazolo[1,5-a]pyridine (0.15 g, 0.4 mmol) in dichloromethane (0.5 mL) was then added dropwise and the reaction stirred for 16 hours. Dichloromethane (20 mL) and water (20 mL) were added and the aqueous phase was separated. The aqueous phase was further extracted with dichloromethane (2×20 mL) and the combined organic phases were dried over anhydrous magnesium sulfate and the solvents evaporated to give a brown solid. Purification on silica gel using 4:1 hexane/ethyl acetate as eluent gave the title compound, 0.111 g (70%). ¹H NMR (CDCl₃): δ 2.59 (s, 3H), 4.60 (t, J=4.1 Hz, 1H), 4.67 (t, J=4.1 Hz, 1H), 4.87 (t, J=4.1 Hz, 1H), 4.98 (t, J=4.1 Hz, 1H), 6.37 (d, J=7.3 Hz, 1H), 6.64 (d, J=5.3 Hz, 1H), 7.13 (t, J=8.6 Hz, 2H), 7.37 (t, J=8.2 Hz, 1H), 7.58 (dd, J=5.3, 8.6 Hz, 2H), 8.15 (d, J=8.8 Hz, 1H), 8.21 (d, J=5.5 Hz, 1H); APESI+MS m/z 399 (M+1)⁻.

### b) 7-Chloro-2-(4-fluorophenyl)-3-(4-(2-methylthio)pyrimidinyl)pyrazolo[1,5-a]pyridine

A solution of 2-(4-fluorophenyl)-3-(4-(2-methylthio)pyrimidinyl)-pyrazolo[1,5-a]pyridine (Example 51, 1.0 g, 3.0 mmol) in tetrahydrofuran (20 mL) was cooled to under nitrogen. lithium diisopropylamide (2M solution in heptane/tetrahydrofuran/ethylbenzene, 3.0 mL, 6.0 mmol) was added dropwise. The solution was stirred for 5 minutes then a solution of toluenesulfonyl chloride (1.2 g, 6.3 mmol) in tetrahydrofuran (5 mL) was added dropwise and the reaction mixture was stirred for 1 hour at −78° C. and then allowed to warm to room temperature. Ethyl acetate (30 mL) and water (20 mL) were added and the organic phase was separated. The aqueous phase was extracted with Ethyl acetate (3×20 mL) and the combined ethyl acetate phases were dried over magnesium sulfate filtered and the solvent was evaporated to give a brown oil. Purification on silica gel using 1:1 hexane/dichloromethane as eluent gave 7-chloro-2-(4-fluorophenyl)-3-(4-(2-methylthio)pyrimidinyl)pyrazolo[1,5-a]pyridine, 0.316 g (28%). ¹H NMR (CDCl₃): δ 2.65 (s, 3H), 6.73 (d, J=5.4 Hz, 1H), 7.12-7.25 (m, 3H), 7.39 (dd, J=7.4, 8.9 Hz, 1H), 7.62-7.69 (m, 2H), 8.30 (d, J=5.2 Hz, 1H), 8.50 (d, J=8.1 Hz, 1H); APEI+MS m/z 371/373 (M+1)⁻.

### EXAMPLE 57

### N-Butyl-4-[7-(2-fluoroethoxy)-2-(4-fluorophenyl)pyrazolo[1,5-a]pyridin-3-yl]-2-pyrimidinamine

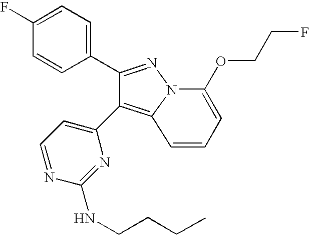

In a similar manner as described in Example 16, from 7-(2-fluoroethoxy)-2-(4-fluorophenyl)-3-(4-(2-methylsulfinyl)pyrimidinyl)pyrazolo[1,5-a]pyridine (Example 71) and n-butylamine was obtained the title compound, (68%). ¹H NMR (CDCl₃): δ 0.96 (t, J=7.3 Hz, 3H), 1.44 (sextet, J=7.5 Hz, 2H), 1.62 (quintet, J=7.5 Hz, 2H), 3.45 (q, J=6.5 Hz, 2H), 4.59 (t, J=4.1 Hz, 1H), 4.66 (t, J=4.1 Hz, 1H), 4.86 (t, J=4.1 Hz, 1H), 4.98 (t, J=4.1 Hz, 1H), 5.4 (broad s, 1H), 6.26 (d, J=5.3 Hz, 1H), 6.32 (d, J=7.3 Hz, 1H), 7.11 (t, J=8.7 Hz, 2H), 7.30 (t, J=7.8 Hz, 1H), 7.60 (dd, J=5.4, 8.6 Hz, 2H), 8.01 (d, J=5.1 Hz, 1H), 8.09 (d, J=9.0 Hz, 1H); APESI+MS m/z 424 (M+1)⁻.

### EXAMPLE 58

### N-Benzyl-4-[7-(2-fluoroethoxy)-2-(4-fluorophenyl)pyrazolo[1,5-a]pyridin-3-yl]-2-pyrimidinamine

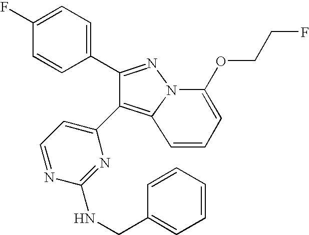

In a similar manner as described in Example 16, from 7-(2-fluoroethoxy)-2-(4-fluorophenyl)-3-(4-(2-methylsulfinyl)pyrimidinyl)pyrazolo[1,5-a]pyridine (Example 70) and benzylamine was obtained the title compound, (73%). ¹H NMR (CDCl₃): δ 4.56 (t, J=4.1 Hz, 1H), 4.64 (t, J=4.1 Hz, 1H), 4.72 (d, J=5.7 Hz, 2H), 4.85 (t, J=4.1 Hz, 1H), 4.96 (t, J=4.1 Hz, 1H), 5.7 (broad s, 1H), 6.28-6.31 (m, 2H), 7.08-7.16 (m, 3H), 7.26-7.30 (m, 1H), 7.34 (d, J=7.9 Hz, 2H), 7.39 (t, J=6.5 Hz, 2H), 7.60 (dd, J=5.5, 8.6 Hz, 2H), 7.75 (broad s, 1H), 8.01 (d, J=5.1 Hz, 1H); APESI+MS m/458 (M+1)⁻.

### EXAMPLE 59

### 2-(4-Fluorophenyl)-3-(4-(2-methylthio)pyrimidinyl)-7-(2,2,2-trifluoroethoxy)pyrazolo[1,5-a]pyridine

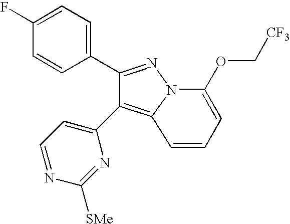

### a) 2-(4-Fluorophenyl)-3-(4-(2-methylthio)pyrimidinyl)-7-(2,2,2-trifluoroethoxy)pyrazolo[1,5-a]pyridine

A solution of 7-chloro-2-(4-fluorophenyl)-3-(4-(2-methylthio)-pyrimidinyl)pyrazolo[1,5-a]pyridine (1.6 g, 4.3 mmol) in dichloromethane (100 mL), was cooled in an ice bath. To this solution was added a solution of 2,2,2-trifluoroethanol (1.6 mL, 22 mmol) and potassium tert-butoxide (22 mL of a 1M solution in tert-butanol) in tetrahydrofuran (50 mL). The reaction mixture was subsequently warmed to 60° C. for 18 h, then poured into cold water and neutralized with 1 N HCl. The phases were separated, and the organics were washed with water (2×50 mL), dried (magnesium sulfate), filtered, and evaporated under reduced pressure. The residue was purified by silica gel chromatography with ethyl acetate:hexane (1:2) to give the title compound as a yellow solid, 1.6 g (86%): ¹H NMR (CDCl₃): δ 2.65 (s, 3H), 4.86 (q, J=8.0 Hz, 2H), 6.56 (d, J=7.4 Hz, 1H), 6.73 (d, J=5.4 Hz, 1H), 7.20 (t, J=8.6 Hz, 2H), 7.42 (t, J=8.3 Hz, 1H), 7.65 (dd, J=5.5 Hz, 8.8 Hz, 2H), 8.28 (d, J=8.9 Hz, 1H), 8.29 (d, J=5.2 Hz, 1H); APESI+MS m/z 435 (M+1).

### b) 7-Chloro-2-(4-fluorophenyl)-3-(4-(2-methylthio)pyrimidinyl)pyrazolo[1,5-a]-pyridine

2-(4-Fluorophenyl)-3-(4-(2-methylthio)pyrimidinyl)pyrazolo[1,5-a]pyridine (17 g, 50 mmol) (see Example 51) was dissolved in tetrahydrofuran and cooled to −78° C. in a dry ice/acetone bath. Lithium diisopropylamide (2M solution in tetrahydrofuran, 76 mL 0.152 mol) was added. After 20 min, carbon tetrachloride (88 mL, 910 mmol) was added. After 2 h, the solution was quenched with saturated brine (50 mL), and layers separated. The organics were washed with saturated brine (100 mL), dried (magnesium sulfate), filtered and concentrated. The residue was purified by silica gel chromatography with dichloromethane to give the title compound as a yellow solid, 159 (80%). ¹H NMR (CDCl₃): δ 2.67 (s, 3H), 4.86 (q, J=8.0 Hz, 2H), 6.57 (d, J=7.4 Hz, 1H), 6.75 (d, J=5.4 Hz, 1H), 7.21 (t, J=8.6 Hz, 2H), 7.45 (t, J=8.2 Hz, 1H), 7.65 (dd, J=5.4 Hz, 8.7 Hz, 2H), 8.28 (apparent d, J=8.1 Hz, 2H); APESI+MS m/z 371 (M+1).

### EXAMPLE 60

### N-Butyl-4-[2-(4-fluorophenyl)-7-(2,2,2-trifluoroethoxy)pyrazolo[1,5-a]pyridin-3-yl]-2-pyrimidinamine

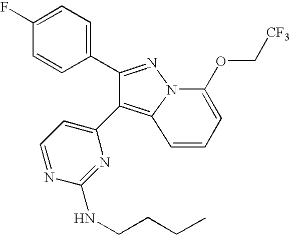

In a similar manner as descibed in Example 16, from 2-(4-fluorophenyl)-3-(4-(2-methylsulfinyl)pyrimidinyl)-7-(2,2,2-trifluoroethoxy)pyrazolo[1,5-a]pyridine (Example 72) and n-butylamine was obtained the title compound as a white solid, (31%). ¹H NMR (CDCl₃): δ 1.02 (t, J=7.3 Hz, 3H), 1.51 (sextet, J=7.5 Hz, 2H), 1.72 (quintet, J=7.5 Hz, 2H), 3.51 (q, J=7.0 Hz, 2H), 4.86 (q, J=8.1 Hz, 2H), 5.2 (broad s, 1H), 6.35 (d, J=5.3 Hz, 1H), 6.52 (d, J=7.2 Hz, 1H), 7.17 (t, J=8.7 Hz, 2H), 7.35 (dd, J=7.6, 8.8 Hz, 2H), 7.68 (dd, J=5.4, 8.6 Hz, 2H), 8.10 (d, J=5.3 Hz, 1H), 8.21 (d, J=8.9 Hz, 1H); APESI+MS m/z 460 (M+1)⁻.

### EXAMPLE 61

### N-Benzyl-4-[2-(4-fluorophenyl)-7-(2,2,2-trifluoroethoxy)pyrazolo[1,5-a]-pyridin-3-yl]-2-pyrimidinamine

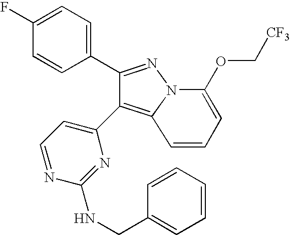

In a similar manner as described in Example 16, from 2-(4-fluorophenyl)-3-(4-(2-methylsulfinyl)pyrimidinyl)-7-(2,2,2-trifluoroethoxy)pyrazolo[1,5-a]pyridine (Example 72, 0.034 g, 0.076 mmol) and benzylamine was obtained the title compound, 0.03 g (80%). ¹H NMR (CDCl₃): δ 4.65 (d, J=5.8 Hz, 2H), 4.72 (dd, J=8.1, 16.3 Hz, 2H), 5.6 (broad s, 1H), 6.27 (d, J=5.3 Hz, 1H), 6.36 (d, J=7.3 Hz, 1H), 7.07 (t, J=8.6 Hz, 3H), 7.23-7.29 (m, 1H), 7.29-7.35 (m, 4H), 7.56 (dd, J=5.7, 8.5 Hz, 2H), 7.7 (broad s, 1H), 8.00 (d, J=5.3 Hz, 1H); APESI+MS m/z 494 (M+1)⁻.

### EXAMPLE 62

### N-Cyclopropyl-4-[2-(4-fluorophenyl)-7-(2,2,2-trifluoroethoxy)pyrazolo [1,5-a]pyridin-3-yl]-2-pyrimidinamine

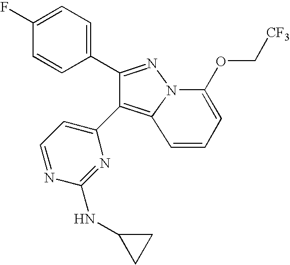

2-(4-Fluorophenyl)-3-(4-(2-methylsulfinyl)pyrimidinyl)-7-(2,2,2-trifluoroethoxy)-pyrazolo[1,5-a]pyridine (Example 72, 1.4 g, 3.1 mmol) was dissolved in dichloromethane (50 mL) and treated with cyclopropylamine (10 mL, 61 mmol). The solution was heated at reflux for six days, cooled to room temperature and then diluted with dichloromethane. The solution was washed with saturated sodium bicarbonate (25 mL) and water (25 mL), dried (magnesium sulfate), filtered and the solvent evaporated under reduced pressure. The residue was purified by silica gel chromatography with ethyl acetate:hexane (1:1) as eluent to give the title compound as a white solid, 1.1 g (80%): ¹H NMR (acetone-d₆): δ 0.47 (br. s, 2H), 0.66 (br. s, 2H), 2.70 (m, 1H), 5.02 (q, J=8.2 Hz, 2H), 6.22 (d, J=5.2 Hz, 1H), 6.41 (br. s, 1H), 6.62 (d, J=7.2 Hz, 1H), 7.15 (t, J=8.5 Hz, 2H), 7.33 (t, J=8.1 Hz, 1H), 7.60 (m, 2H), 7.94 (d, J=5.1 Hz, 1H), 8.34 (br. s, 1H); APESI+MS m/z 444 (M+1).

### EXAMPLE 63

### N-Cyclopentyl-4-[2-(4-fluorophenyl)-7-(2,2,2-trifluoroethoxy)pyrazolo[1,5-a]pyridin-3-yl]-2-pyrimidinamine

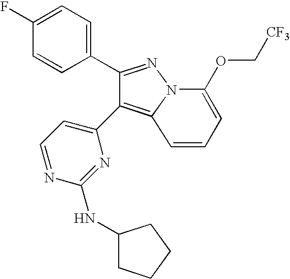

2-(4-Fluorophenyl)-3-(4-(2-methylsulfinyl)pyrimidinyl)-7-(2,2,2-trifluoroethoxy)-pyrazolo[1,5-a]pyridine (Example 72, 0.05 g, 0.11 mmol) was dissolved in cyclopentylamine (1 mL) and heated at 60° C. for 18 hours. The reaction mixture was diluted with ethyl acetate (40 mL) and extracted with water (2×10 mL). The organic layer was dried (magnesium sulfate), filtered and the solvent was evaporated under reduced pressure. The residue was purified on a silica gel preparative chromatography plate (2 mm) with ethyl acetate:hexanes (1:2) as eluent to give the title compound, 0.008 g (15%). ¹H NMR (acetone-d₆): δ 1.60 (m, 4H), 1.75 (m, 2H), 2.04 (m, 2H), 4.32 (m, 1H), 5.13 (q, J=8.4 Hz, 2H), 6.23 (br. s, 1H), 6.30 (d, J=4.7 Hz, 1H), 6.73 (d, J=7.5 Hz, 1H), 7.25 (t, J=8.7 Hz, 2H), 7.43 (t, J=8.1 Hz, 1H), 7.70 (dd, J=5.5 Hz, 8.8 Hz, 2H), 8.04 (d, J=5.1 Hz, 1H), 8.24 (d, J=9 Hz, 1H); APESI+MS m/472 (M+1).

### EXAMPLE 64

### N-Cyclohexyl-4-[2-(4-fluorophenyl)-7-(2,2,2-trifluoroethoxy)pyrazolo[1,5-a]pyridin-3-yl]-2-pyrimidinamine

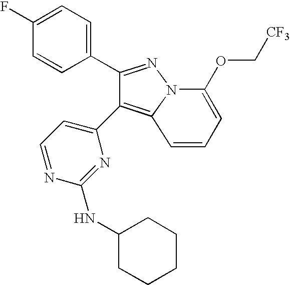

In a similar manner as described in Example 63 from 2-(4-fluorophenyl)-7-(2,2,2-trifluoroethoxy)-3-(4-(2-methylsulfinyl)pyrimidinyl)pyrazolo[1,5-a]pyridine (Example 72) was obtained the title compound, (49%). ¹H NMR (acetone-d₆): 81.3 (m, 5H), 1.60 (m, 1H), 1.80 (m, 2H), 2.00 (m, 2H), 3.80 (m, 1H), 5.13 (q, J=8.4 Hz, 2H), 6.12 (s, 1H), 6.30 (s, 1H), 6.73 (d, J=7.5 Hz, 1H), 7.25 (t, J=8.7 Hz, 2H), 7.43 (t, J=8.1 Hz, 1H), 7.69 (dd, J=5.5 Hz, 8.6 Hz, 2H), 8.04 (d, J=5.1 Hz, 1H), 8.22 (d, J=8.9 Hz, 1H); APESI+MS m/z 485 (M+1).

### EXAMPLE 65

### 3-(4-[2-(4-Fluorophenyl)-7-(2,2,2-trifluoroethoxy)pyrazolo[1,5-a]pyridin-3-yl]-2-pyrimidinylamino)-1-propanol

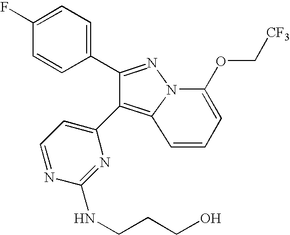

In a similar manner as described in Example 63. From 2-(4-fluorophenyl)-7-(2,2,2-trifluoroethoxy)-3-(4-(2-methylsulfinyl)pyrimidinyl)pyrazolo[1,5-a]pyridine (Example 72) was obtained the title compound, (38%). ¹H NMR (acetone-d₆): δ 1.69 (m, 2H), 3.44 (apparent q, J=6.4 Hz, 2H), 3.53 (s, 2H), 3.75 (br. s, 1H), 5.01 (q, J=8.4 Hz, 2H), 6.19 (d, J=5.2 Hz, 1H), 6.32 (br. s, 1H), 6.62 (d, J=7.5 Hz, 1H), 7.14 (t, J=8.9 Hz, 2H), 7.32 (t, J=8.2 Hz, 1H), 7.59 (dd, J=5.6 Hz, 8.6 Hz, 2H), 7.93 (d, J=5.2 Hz, 1H), 8.14 (d, J=8.9 Hz, 1H); APESI+MS m/z 462 (M+1).

### EXAMPLE 66

### 2-(4-Fluorophenyl)-3-(4-(2-methyloxy)pyrimidinyl)-7-(2,2,2-trifluoro ethoxy)pyrazolo[1,5-a]pyridine

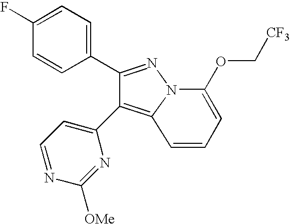

2-(4-Fluorophenyl)-7-(2,2,2-trifluoroethoxy)-3-(4-(2-methylsulfinyl)pyrimidinyl) pyrazolo[1,5-a]pyridine (Example 72) (0.05 g, 0.11 mmol) was dissolved in 2 N ammonia in methanol (20 mL) and the mixture was heated at 80° C. for 18 hours. The reaction mixture was diluted with ethyl acetate (40 mL) and extracted with water (2×10 mL). The organic layer was dried (magnesium sulfate), filtered and evaporated under reduced pressure. The residue was purified on a silica gel preparative chromatography plate (2 mm) with ethyl acetate:hexane (1:2) as eluent to give the title compound, 0.034 g (73%). ¹H NMR (CDCl₃) δ 4.12 (s, 3H), 4.86 (q, J=8.0 Hz, 2H), 6.56 (d, J=7.4 Hz, 1H), 6.71 (d, J=5.2 Hz, 1H), 7.21 (t, J=8.5 Hz, 2H), 7.42 (t, J=8.2 Hz, 1H), 7.65 (dd, J=5.4 Hz, 7.7 Hz, 2H), 8.30 (d, J=5.2 Hz, 1H), 8.35 (d, J=8.9 Hz, 1H); APESI+MS m/z 419 (M+1).

### EXAMPLE 67

### 2-(4-Fluorophenyl)-3-(4-(2-phenyloxy)pyrimidinyl)-7-(2,2,2-trifluoro ethoxy)pyrazolo[1,5-a]pyridine

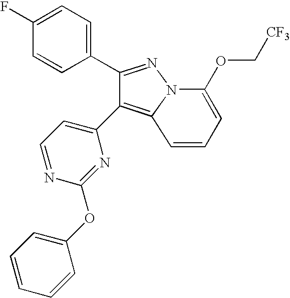

2-(4-Fluorophenyl)-7-(2,2,2-trifluoroethoxy)-3-(4-(2-methylsulfinyl)pyrimidinyl) pyrazolo[1,5-a]pyridine (Example 72) (0.05 g, 0.11 mmol) was dissolved in dichloromethane (3 mL) and treated with a solution of phenol (0.1 mL, 1.1 mmol) and potassium tert-butoxide (1.2 mL of a 1 N in tert-butyl alcohol) in tetrahydrofuran (3 mL). After 30 min at ambient temperature, the reaction was quenched with water and diluted with ethyl acetate (40 mL) and extracted with water (2×10 mL). The organic layer was dried (magnesium sulfate), filtered and evaporated under reduced pressure. The residue was purified on a silica gel preparative chromatography plate (2 mm) with ethyl acetate:hexane (1:2) as eluent to give the title compound, 0.032 g (61%). ¹H NMR (CDCl₃): δ 4.81 (q, J=8.0 Hz, 2H), 6.49 (d, J=7.4 Hz, 1H), 6.74 (d, J=5.4 Hz, 1H), 7.17 (t, J=7.7 Hz, 1H), 7.23 (t, J=8.6 Hz, 2H), 7.32 (d, J=7.7 Hz, 2H), 7.36 (t, J=7.4 Hz, 1H), 7.53 (t, J=7.7 Hz, 2H), 7.63 (dd, J=5.4 Hz, 8.6 Hz, 2H), 7.76 (d, J=8.8 Hz, 1H), 8.31 (d, J=5.4 Hz, 1H); APESI+MS m/z 481 (M+1).

### EXAMPLE 68

### 2-(4-Fluorophenyl)-3-(4-(2-(2,2,2-trifluoroethoxy))pyrimidinyl)-7-(2,2,2-trifluoroethoxy)pyrazolo[1,5-a]pyridine

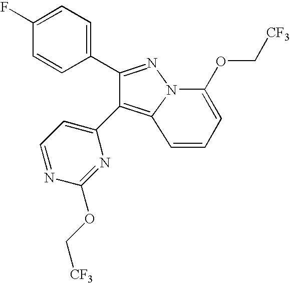

In a similar manner as described in Example 56, from 2-(4-fluorophenyl)-3-(4-(2-methylsulfinyl)pyrimidinyl)-7-(ethylsulfinyl)pyrazolo[1,5-a]pyridine (Example 69) and 2,2,2-trifluoroethanol was obtained the title compound, (10%). ¹H NMR (CDCl₃): δ 4.78-4.85 (m, 4H), 6.52 (d, J=7.3 Hz, 1H), 6.75 (d, J=5.3 Hz, 1H), 7.16 (t, J=8.6 Hz, 2H), 7.41 (t, J=8.2 Hz, 1H), 7.58 (dd, J=5.3, 8.6 Hz, 2H), 8.22 (d, J=8.8 Hz, 1H), 8.25 (d, J=5.3 Hz, 1H); APESI+MS m/z 487 (M+1)⁻.

### EXAMPLE 69

### 2-(4-Fluorophenyl)-3-(4-(2-methylsulfinyl)pyrimidinyl)-7-(ethylsulfinyl)-pyrazolo[1,5-a]pyridine

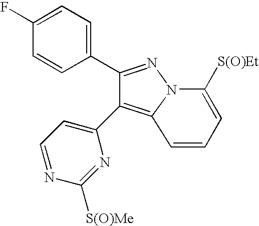

In a similar manner as described in Example 55, from 2-(4-fluorophenyl)-3-(4-(2-methylthio)pyrimidinyl)-7-(ethylthio)pyrazolo[1,5-a]pyridine was obtained the title compound. ¹H NMR (CDCl₃): δ 1.25 (t, J=7.5 Hz, 3H), 3.00 (s, 3H), 3.33-3.45 (m, 2H), 7.05 (d, J=5.5 Hz, 1H), 7.20 (t, J=8.6 Hz, 2H), 7.54-7.59 (m, 3H), 7.67 (dd, J=7.3, 8.8 Hz, 1H), 8.52 (d, J=5.5 Hz, 1H), 8.25 (t, J=8.4 Hz, 1H); APESI+MS m/z 429 (M+1)⁻.

### EXAMPLE 70

### 2-(4-Fluorophenyl)-3-(4-(2-methylthio)pyrimidinyl)-7-(ethylthio)-pyrazolo[1,5-a]pyridine

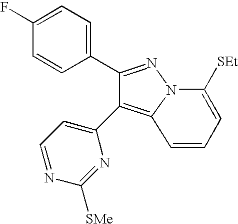

In a similar manner as described in Example 56, from 2-(4-fluorophenyl)-3-(4-(2-methylthio)pyrimidinyl)-pyrazolo[1,5-a]pyridine and diethyl disulfide in place of toluenesulfonyl chloride was obtained 2-(4-fluorophenyl)-3-(4-(2-methylthio)pyrimidinyl)-7-(ethylthio)pyrazolo[1,5-a]pyridine. ¹H NMR (CDCl₃): δ 1.46 (t, J=7.4 Hz, 3H), 2.60 (s, 3H), 3.16 (q, J=7.4 Hz, 2H), 6.67 (d, J=5.5 Hz, 1H), 6.81 (d, J=7.3 Hz, 1H), 7.13 (t, J=8.6 Hz, 2H), 7.35 (t, J=8.1 Hz, 1H), 7.59 (dd, J=5.5, 8.6 Hz, 2H), 8.22 (d, J=5.5 Hz, 1H), 8.28 (d, J=9.0 Hz, 1H).

### EXAMPLE 71

### 7-(2-Fluoroethoxy)-2-(4-fluorophenyl)-3-(4-(2-methylsulfinyl)-pyrimidinyl)-pyrazolo[1,5-a]pyridine

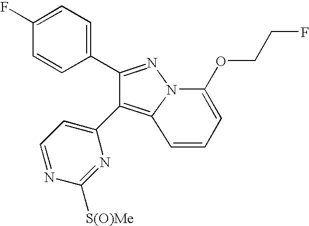

In a similar manner as described in Example 55, from 7-(2-fluoroethoxy)-2-(4-fluorophenyl)-3-(4-(2-methylthio)pyrimidinyl)pyrazolo[1,5-a]pyridine was obtained 7-(2-fluoroethoxy)-2-(4-fluorophenyl)-3-(4-(2-methylsulfinyl)pyrimidinyl)pyrazolo[1,5-a]pyridine. ¹H NMR (CDCl₃): δ 3.04 (s, 3H), 4.65 (t, J=4.0 Hz, 1H), 4.75 (t, J=4.0 Hz, 1H), 4.91 (t, J=4.1 Hz, 1H), 5.06 (t, J=4.1 Hz, 1H), 6.50 (d, J=7.5 Hz, 1H), 7.02 (d, J=5.5 Hz, 1H), 7.24 (t, J=8.6 Hz, 2H), 7.54 (t, J=8.3 Hz, 1H), 7.62-7.67 (m, 2H), 8.48-8.53 (m, 2H).

### EXAMPLE 72

### 2-(4-Fluorophenyl)-3-(4-(2-methylsulfinyl)pyrimidinyl)-7-(2,2,2-trifluoroethoxy)-pyrazolo[1,5-a]pyridine

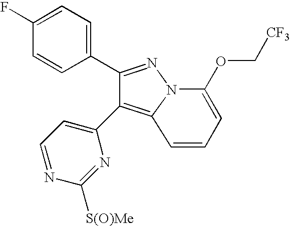

In a similar manner as described in Example 55, from 2-(4-fluorophenyl)-3-(4-(2-methylthio)pyrimidinyl)-7-(2,2,2-trifluoroethoxy)pyrazolo[1,5-a]pyrid ine and m-chloroperbenzoic acid was obtained 2-(4-fluorophenyl)-3-(4-(2-methylsulfinyl)-pyrimidinyl)-7-(2,2,2-trifluoroethoxy)pyrazolo[1,5-a]pyridine. ¹H NMR (CDCl₃): δ 3.03 (s, 3H), 4.85 (q, J=8.0 Hz, 2H), 6.60 (d, J=7.3 Hz, 1H), 7.05 (d, J=5.3 Hz, 1H), 7.24 (t, J=7.6 Hz, 2H), 7.52 (t, J=8.2 Hz, 2H), 7.60-7.68 (m, 2H), 8.51 (d, J=5.5 Hz, 1H), 8.57 (d, J=8.8 Hz, 1H).

### EXAMPLE 73

### 2-(4-Fluorophenyl)-3-(4-pyridyl)-pyrazolo[1,5-a]pyridine

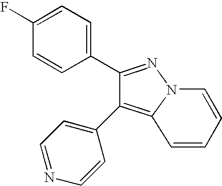

### a) 2-(4-Fluorophenyl)-3-(4-pyridyl)-pyrazolo[1,5-a]pyridine

To a solution of 2-(4-fluorophenyl)-3-bromopyrazolo[1,5-a]pyridine (0.2 g, 0.68 mmol) and 4-(tributylstannyl)pyridine (0.38 g, 1 mmol) in dry toluene (10 mL) was added tetrakis(triphenylphosphine)palladium (0) (0.03 g, 0.03 mmol) and the mixture was heated at reflux temperature under a nitrogen atmosphere for about 48 hours. The mixture was cooled to room temperature and diluted with diethyl ether (40 mL). The mixture was poured into a 10% aqueous solution of potassium fluoride (20 mL) and the mixture was stirred for 1 hour. The biphasic mixture was filtered through a pad (1 cm) of diatomaceous earth and the organic phase was separated. The aqueous phase was extracted with diethyl ether (10 mL) and the combined organic phases are washed with brine, dried over anhydrous magnesium sulfate, filtered and the solvent evaporated under reduced pressure. The residue was purified using silica gel chromatography with 20% ethyl acetate in hexanes, followed by 50% ethyl acetate in hexanes, as eluent to give the title compound as an off white solid, 0.16 g (80%). ¹H NMR (CDCl₃) δ 8.58 (s, 2H), 8.50 (d, 1H, J=7.2 Hz), 7.63 (d, 1H, 9 Hz), 7.52 (m, 2H), 7.27-7.20 (m, 3H), 7.06 (t, 2H, 8.7 Hz), 6.86 dt, 1H, J=7, 1 Hz). MS (+ve ion electrospray) 290 (100), (MH⁺).

### b) 2-(4-Fluorophenyl)-3-bromopyrazolo[1,5-a]pyridine

To a solution of 2-(4-fluorophenyl)-pyrazolo[1,5-a]pyridine-3-carboxylic acid (0.96 g, 3.75 mmol) in dry N,N-dimethylformamide (10 mL) was added sodium bicarbonate (0.95 g, 11.3 mmol) followed by N-bromosuccinimide (0.667 g, 3.75 mmol) and the mixture was stirred at room temperature under a nitrogen atmosphere for about 90 minutes. The mixture was poured into water (300 mL) and the resulting solid was collected by filtration and washed with water. The solid was dissolved in 10:1 chloroform:methanol (10 mL) and filtered through a pad (0.5 cm) of silica gel using 10:1 chloroform:methanol as eluent. The filtrate was evaporated to leave the title compound as a tan solid, 0.87 g (80%). ¹H NMR (d6 DMSO) δ 8.7 (d, 1H, J=6.9 Hz), 8.02 (dd, 2H, J=8.7, 5.7 Hz), 7.61 (d, 1H, J=8.4 Hz), 7.40 (t, 1H, J=6 Hz), 7.38 (t, 2H, J=9 Hz), 7.04 (t, 1H, J=6.9 Hz). MS (+ve ion electrospray) 293 (100), (MH⁺).

### c) 2-(4-Fluorophenyl)-pyrazolo[1,5-a]pyridine-3-carboxylic Acid

A solution of methyl 2-(4-fluorophenyl)-pyrazolo[1,5-a]pyridine-3-carboxylate (5.0 g, 18.5 mmol) in 2N aqueous sodium hydroxide (50 ml) and methanol (30 mL) was heated at reflux for about 3 hours. The mixture was filtered and the filtrate was washed with diethyl ether (20 mL) and then concentrated under reduced pressure to about half the original volume. Concentrated hydrochloric acid was added to adjust the pH to about 2 and the resulting solid was collected by filtration and washed with water and dried under vacuum to give the title compound as a white solid, 4.8 g (ca. 100%). ¹H NMR (d6 DMSO) δ 12.43 (s, 1 h), 8.84 (d, 1H, J=6.9 Hz), 8.14 (d, 1H, J=9 Hz), 7.82 (m, 2H), 7.57 (t, 1H, J=8.1 Hz), 7.28 (t, 2H, J=9 Hz), 7.15 (td, 1H, J=6.9, 1.2 Hz). MS (+ve ion electrospray) 257 (100), (MH⁺).

### d) Methyl 2-(4-fluorophenyl)-pyrazolo[1,5-a]pyridine-3-carboxylate

A stirred solution of methyl 3-(4-fluorophenyl)propiolate (8.02 g, 45 mmol) and 1-aminopyridinium iodide (10 g, 45 mmol) in dry acetonitrile (150 mL) was cooled to about 0° C. A solution of 1,8-diazabicycloundec-7-ene (13.7 g, 90 mmol) in dry acetonitrile (50 mL) was added dropwise over 1 hour. The mixture was allowed to stir at room temperature for about 18 h. The reaction mixture was cooled in an ice bath for about 30 minutes and the precipitate was collected by filtration and washed with cold acetonitrile (10 mL). The solid was dried under reduced pressure to give the title compound as a white solid, 8.48 g (70%). ¹H NMR (CDCl₃) δ 8.50 (d, 1H, J=8.4 Hz), 8.18 (d, 1H, J=8.8 Hz), 7.78 (m, 2H), 7.42 (t, 1H, J=8.4 Hz), 7.13 (t, 2H, J=8.8 Hz), 6.97 (td, 1H, J=6.8, 1 Hz). MS (+ve ion electrospray) 271 (100), (MH⁺).

### e) Methyl 3-(4-fluorophenyl)propiolate

A solution of 1-(4-fluorophenyl)-2-trimethylsilylacetylene (64 g, 0.33 mol) in dry diethyl ether (400 mL) was cooled to 0° C. under a nitrogen atmosphere. To this solution was added, dropwise over 45 minutes, a solution of tetrabutylammonium fluoride (1M in tetrahydrofuran, 330 mL, 0.33 mol) via a dropping funnel maintaining the internal temperature below 2° C. The mixture was allowed to warm to room temperature over about 1 hour. Diethyl ether (300 mL) was added to the mixture and the organic solution was washed with water, saturated brine and then dried over anhydrous magnesium sulfate. The magnesium sulfate was removed by filtration and the filtrate was cooled to about −78° C. n-Butyl lithium (1.6M in hexanes, 450 mL, 0.72 mol) was added dropwise via a dropping funnel over about 1 hour while the temperature was maintained below −66° C. After complete addition the mixture was stirred at −78° C. for about 1 hour and then a precooled solution of methyl chloroformate (110 mL, 1.4 mol) in dry diethyl ether (200 mL) was added in a continuous stream as fast as possible. The mixture was allowed to cool to −78° C. and the allowed to warm to room temperature over 1.5 h. The organic reaction mixture was washed with water and saturated brine and then dried over anhydrous magnesium sulfate. The solvents are remove under reduced pressure and the residue dried under reduced pressure to give the title compound as a brown solid, 36.5 g (61%). ¹H NMR (CDCl3) δ 7.58 (dd, 2H, J=9, 5.4 Hz), 7.07 (t, 2H, J=8.5 Hz), 3.84 (s, 3H). MS (+ve ion electrospray) 178 (30), (M⁺).

### f) 1-(4-Fluorophenyl)-2-trimethylsilylacetylene

4-Fluoroiodobenzene (112 mL, 0.97 mol) and triethylamine (176 mL, 1.26 mol) are dissolved in dry tetrahydrofuran (1.2 L) and nitrogen gas was bubbled through the solution for about 20 min. Copper (I) iodide (1.08 g, 5.7 mmol) and bis(triphenyphosphine)palladium dichloride (2.15 g, 3 mmol) are added and then trimethylsilylacetylene (178 mL, 1.3 mol) was added dropwise over about 40 min with the temperature being maintained at about 23° C. A large amount of precipitate forms (presumably Et₃NHCl) which necessitates mechanical stirring. Following complete addition of the trimethylsilylacetylene the mixture was allowed to stir at room temperature for about 18 hours. The mixture was filtered and the solid washed with cyclohexane. The combined filtrates are concentrated under reduce pressure to give a brown oil. Application of this oil to a pad of silica gel followed by elution with cyclohexane gave a yellow solution. Removal of the solvent gave the title compound as a yellow oil; 182.8 g (95%).

### EXAMPLE 74

### 2-(4-Fluorophenyl)-7-methyl-3-(4-pyridinyl)pyrazolo[1,5-a]pyridine

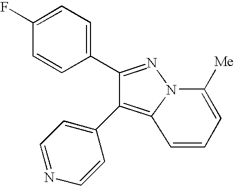

### a) 2-(4-Fluorophenyl)-7-methyl-3-(4-pyridinyl)pyrazolo[1,5-a]pyridine

In a similar manner as described in Example 73, from 2-(4-fluorophenyl)-3-bromo-7-methylpyrazolo[1,5-a]pyridine (0.1 g, 0.33 mmol) and 4-(tri-n-butyl)stannylpyridine (0.17 g, 0.46 mmol) was obtained the title compound as a white solid, 0.016 g (140/%). This material was dissolved in diethyl ether and treated with HCl in diethyl ether to afford the corresponding hydrochloride salt. ¹H NMR (DMSO-d6) δ 8.74 (d, 2H, J=6.6 Hz), 7.91 (d, 1H, J=8.9 Hz), 7.81 (d, 2H, J=6.6 Hz), 7.61 (m, 2H), 7.56 (t, 1H, J=15.9 Hz), 7.34 (t, 2H, J=17.6 Hz), 7.15 (d, 1H, J=6.9 Hz), 2.79 (s, 3H). MS (+ve electrospray) 303 (100), (M+).

### b) 2-(4-Fluorophenyl)-3-bromo-7-methyl-pyrazolo[1,5-a]pyridine

Following the procedure outlined in Example 73, from 2-(4-fluorophenyl)-7-methyl-pyrazolo[1,5-a]pyridine-3-carboxylic acid was obtained the title compound, ¹H NMR (CDCl₃) δ 8.00 (m, 2H), 7.38 (d, 1H, J=8.8 Hz), 7.11 (m, 3H), 6.62 (d, 1H, J=6.9 Hz), 2.71 (s, 3H). MS (+ve electrospray) 306 (25), (MH⁺).

### c) 2-(4-Fluorophenyl)-7-methyl-pyrazolo[1,5-a]pyridine-3-carboxylic Acid

In a similar manner as described in Example 73, from methyl 2-(4-fluorophenyl)-7-methyl-pyrazolo[1,5-a]pyridine-3-carboxylate was obtained the title compound as a white solid, ¹H NMR (DMSO-d6) δ 8.08 (d, 1H, J=8.8 Hz), 7.84 (m, 2H), 7.76 (m, 1H), 7.53 (m, 1H), 7.30 (t, 2H, J=17.8 Hz), 7.09 (d, 1H, J=6.8 Hz), 2.75 (s, 3H). MS (+ve electrospray) 270 (100), (M+).

### d) Methyl 2-(4-fluorophenyl)-7-methyl-pyrazolo[1,5-a]pyridine-3-carboxylate

To a stirred solution of methyl 3-(4-fluorophenyl)propiolate (3.47 g, 19.5 mmol) and 1-amino-2-methylpyridinium 2,4,6-trimethylbenzenesulfonate (6.0 g, 19.5 mmol) in dry acetonitrile (75 mL) was added, dropwise over 10 min a solution of 1,8-diazabicycloundec-7-ene (5.82 mL, 39 mmol) in dry acetonitrile (25 mL). The mixture was allowed to stir at room temperature for about 18 hours. The solvent was evaporated under reduced pressure and the residue was partitioned between water (500 mL) and ethyl acetate (250 mL) and the organic phase separated. The aqueous was extracted with ethyl acetate and the combined organic extracts are dried over anhydrous magnesium sulfate, and the solvent removed under vacuum. The residue was purified by chromatography on silica gel using 10:1 hexanes:ethyl acetate as eluent to give the title compound as a white solid, 4.65 g (86%). ¹H NMR (CDCl₃) δ 8.15 (d, 1H, J=8.8 Hz), 7.86 (m, 2H), 7.41 (t, ₁H, J=8.9 Hz), 7.19 (t, 2H, J=17.6 Hz), 6.87 (d, 1H, J=7.0 Hz), 3.89 (s, 3H), 2.85 (s, 3H). MS (+ve ion electrospray) 285 (100), (MH⁺).

### e) 1-Amino-2-methylpyridinium 2,4,6-trimethylbenzenesulfonate

To cold (0° C.) trifluoroacetic acid (50 mL) was added N-tert-butoxycarbonyl-O-(mesitylsulfonyl)hydroxylamine (16.09 g, 51 mmol) in portions over about 15 minutes. The solution was then stirred for about 15 minutes at room temperature. The solution was poured into ice water (250 mL) and the resulting white precipitate was collected by filtration and air-dried for 5 minutes. The solid was dissolved in chloroform (100 mL) and this solution was dried over anhydrous magnesium sulfate. The magnesium sulfate was removed by filtration and the filtrate was added to a solution of 2-picoline (5.0 g, 54 mmol) in chloroform (5 mL). The mixture was stirred for 45 min and then filtered. To the filtrate was added diethyl ether (225 mL) and the product allowed to precipitate. The solid was collected by filtration, washed with diethyl ether (50 mL) and dried to give the title compound as a white solid, 12.9 g (820/%). ¹H NMR (CDCl₃) δ 9.45 (d, 1H), 8.4 (s, 2H), 7.84 (t, 1H), 7.55 (t, 1H), 7.50 (d, 1H), 6.80 (s, 2H), 2.81 (s, 3H), 2.62 (s, 6H), 2.25 (s, 3H). MS (+ve electrospray) 109 (100), (M⁺).

### EXAMPLE 75

### 2-(4-Fluorophenyl)-7-methoxy-3-(4-pyridinyl)pyrazolo[1,5-a]-pyridine

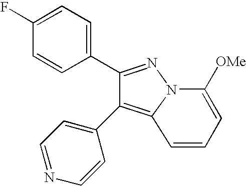

In a similar manner as described in Examples 73 and 74, from 2-methoxypyridine was obtained the title compound. ¹H NMR (400 MHz, DMSO-d₆) δ 4.12 (s, 3H), 6.52 (d, 1H, J=7.2 Hz), 7.24 (m, 4H), 7.35 (m, 2H), 7.51 (dd, 2H, J=5.6 Hz, 8.8 Hz), 8.53 (d, 2H, J=6.0 Hz). MS (ES+) m/320 (M⁺+H).

Alternatively, 2-(4-fluorophenyl)-7-methoxy-3-(4-pyridinyl)pyrazolo[1,5-a]-pyridine can be prepared from 7-chloro-2-(4-fluorophenyl)-3-(4-pyridinyl)pyrazolo[1,5-a]pyridine (see Example 76) by the following procedure: 7-chloro-2-(4-fluorophenyl)-3-(4-pyridinyl)pyrazolo[1,5-a]pyridine (0.05 g, 0.15 mmol) was added to a solution of sodium methoxide (0.75 mmol) in dry methanol (5 mL) and the mixture was heated at reflux for about 24 hours. Water was added and the mixture was extracted with ethyl acetate. The combined organic extracts are washed with brine and dried over anhydrous magnesium sulfate. The solution was filtered through a short pad of silica gel and the solvent was evaporated under vacuum. The residue was purified by silica gel chromatography using 1:10 MeOH:Ethyl acetate to give the title compound, 0.039 g (80%). ¹H NMR and MS are identical to those described above.

### EXAMPLE 76

### 7-Chloro-2-(4-fluorophenyl)-3-(4-pyridinyl)pyrazolo[1,5-a]-pyridine

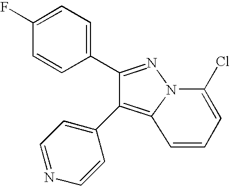

A stirred solution of 2-(4-fluorophenyl)-3-(4-pyridinyl)-pyrazolo[1,5-a]pyridine (from Example 73, 100 mg, 0.346 mmol) in dry tetrahydrofuran (4 mL) was cooled to about −78° C. under N₂ and n-butyllithium in hexanes (2.5 M in hexanes, 0.27 mL, 0.7 mmol) was added dropwise. The mixture was stirred at −78° C. for about 30 min and a solution of p-toluenesulfonyl chloride (0.15 g, 0.76 mmol) in dry tetrahydrofuran (1 mL) was added. The mixture was allowed to warm to room temperature over 30 min and was stirred at room temperature for 1 hour. Water was added and the mixture was poured into a separatory funnel. The organic layer was separated, and the aqueous layer was extracted with ethyl acetate. The combined organic layers were dried using anhydrous magnesium sulfate, filtered and evaporated. Purification by silica gel chromatography yielded the title compound, 0.087 g (78.60/%). ¹H NMR (CDCl₃): δ 8.65 (d, 2H, J=5.8 Hz), 7.55-7.69 (m, 3H), 7.30 (d, 2H, J=5.8 Hz), 7.11-7.21 (m, 1H), 7.04-7.13 (m, 3H). MS (ES+ve): 326 (25, M+3), 323 (50, M⁺), 290 (100).

### EXAMPLE 77

### 2-(4-Fluorophenyl)-3-(2-fluoro-4-pyridinyl)-7-methoxypyrazolo[1,5-a]pyridine

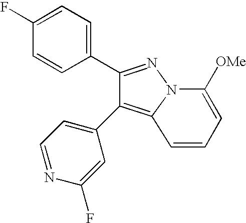

A solution of 3-bromo-2-(4-fluorophenyl)-7-methoxypyrazolo[1,5-a]pyridine (from Example 74, 180 mg, 0.560 mmol), 2-fluoropyridin-4-ylboronic acid (from Example 80, 112 mg, 0.800 mmol) and dichlorobis(triphenylphosphine)palladium (40.0 mg, 0.056 mmol) in N,N-dimethylformamide (6.00 mL) was placed in a pre-heated oil bath at 110° C. To the reaction was added, in a dropwise manner, 2M sodium carbonate (0.840 mL, 1.68 mmol). The reaction was allowed to stir for three hours before cooling to room temperature and filtering through a Celite 545 pad. The Celite filter was washed with ethyl acetate and the filtrate was concentrated to dryness at 50° C. under vacuum. The residue was dissolved in methylene chloride and dried over anhydrous magnesium sulfate. The drying agent was removed by filtration and the filtrate was concentrated and purified by silica gel chromatography to yield the title compound (110 mg, 0.326 mmol, 580/%). ¹H NMR (CDCl₃): δ 8.14(d, 1H, J=5.5 Hz), 7.53(dd, 2H, J=6.0, 8.0 Hz), 7.24-7.32(m, 2H), 7.00-7.10(m, 3H), 6.89(s, 1H), 6.23(dd, 1H. J=2.0, 6.0 Hz), 4.2(s, 3H). MS (ES+ve): 338.1 (40, M⁺), 323.1 (100).

### EXAMPLE 78

### N-Butyl-4-[2-(4-fluorophenyl)-7-methoxypyrazolo[1,5-a]pyridin-3-yl]-2-pyridinamine

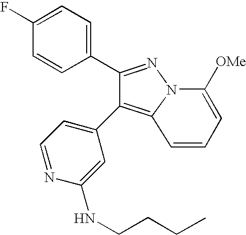

In a sealed-tube was combined 2-(4-Fluorophenyl)-3-(2-fluoro-4-pyridinyl)-7-methoxypyrazolo[1,5-a]pyridine (from Example 77, 20 mg, 0.06 mmol) and n-butylamine (2.0 mL, 1.5 g, 20 mmol), and the reaction was placed in a pre-heated oil bath at 130° C. The reaction was stirred at 130° C. until consumption of starting material was indicated by TLC analysis (50% ethyl acetate in hexanes). The contents of the sealed-tube was transferred to a flask and concentrated to dryness at 50° C. under high vacuum. The residue was purified by silica gel chromatography to yield the title compound, 2.0 mg (0.005 mmol, 8%). ¹H NMR (d₆-acetone): δ 8.04(d, 1H, J=5.1 Hz), 7.74(dd, 2H, J=5.7, 9.0 Hz), 7.33-7.38(m, 2H), 7.22(t, 2H, J=9.0 Hz), 6.45-6.54(m, 3H), 4.25(s, 3H), 3.30-3.40(m, 2H), 1.60(quint, 2H, J=7.2 Hz), 1.45(sext, 2H, J=7.2 Hz), 0.9(t, 3H, J=7.2 Hz). MS (ES+ve): 391.1 (100, M⁺), 376.3 (100).

### EXAMPLE 79

### N-{4-[5-Chloro-7-(ethylsulfanyl)-2-(4-fluorophenyl)pyrazolo[1,5-a]pyridin-3-yl]-2-pyrimidinyl}-N-cyclopentylamine

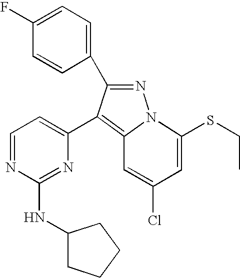

### a) 2-(4-Chloro-2-pyridinyl)-1-(4-fluorophenyl)ethanone

To a cold (0° C.) solution of 4-chloro-2-picoline (5.0 g, 39 mmol) and ethyl 4-fluorobenzoate (6.6 g, 39 mmol) in tetrahydrofuran (100 mL) was added lithium bis(trimethylsilyl)amide (80 mL, 1.0 M in tetrahydrofuran, 80 mmol) dropwise via a pressure equalizing funnel over 30 minutes. Upon complete addition, the cold bath was removed and the resulting solution was stirred at room temperature for 15 hours. The reaction mixture was concentrated under reduced pressure and methanol was added to the reaction, resulting in the formation of a white precipitate. The precipitate was collected by filtration and dried to give 2-(4-chloro-2-pyridinyl)-1-(4-fluorophenyl)ethanone (9.6 g, 99%) as a white solid. ¹H-NMR (DMSO-d₆): δ 7.90 (m, 3H), 7.11 (t, 2H), 6.56 (s, 1H), 5.67 (s, 1H), 4.14 (m, 2H); ¹⁹F-NMR (DMSO-d₆): δ 115.67; MS m/z 250 (M+1).

### b) 2-(4-Chloro-2-pyridinyl)-1-(4-fluorophenyl)ethanone oxime

To a solution of 2-(4-chloro-2-pyridinyl)-1-(4-fluorophenyl)ethanone (9.6 g, 38 mmol) in methanol (200 mL) was added hydroxylamine hydrochloride (13.5 g, 190 mmol) followed by the addition of a sodium hydroxide solution (7.8 g, 190 mmol in 50 mL of water). The resulting suspension was heated at reflux for 2 hours and then allowed to cool to room temperature. The mixture was concentrated and water was added to the resulting slurry. A white precipitate formed, which was collected by filtration, washed with water and dried (magnesium sulfate) to give 2-(4-chloro-2-pyridinyl)-1-(4-fluorophenyl)ethanone oxime (8.45 g, 840/%) as a white solid. ¹H-NMR (DMSO-d₆): δ 11.56 (s, 1H), 8.44 (d, 1H), 7.80 (m, 2H), 7.40 (m, 2H), 7.22 (m, 2H), 4.29 (s, 2H); ¹⁹F-NMR (DMSO-d₆): 8113.44; MS m/z 265 (M+1).

### c) 5-Chloro-2-(4-fluorophenyl)pyrazolo[1,5-a]pyridine

To a solution of 2-(4-chloro-2-pyridinyl)-1-(4-fluorophenyl)ethanone oxime (8.0 g, 30 mmol) in 1,2-dimethoxyethane (50 mL) at 0° C. was added trifluoroacetic anhydride (6.3 g, 30 mmol), keeping the temperature below 10° C. during the addition. After the addition was complete, the reaction was warmed to room temperature. The solution was then cooled to 4° C. and a solution of triethylamine (8.4 mL, 60 mmol) in 1,2-dimethoxyethane (20 mL) was added over a period of 0.5 hours. The mixture was allowed to warm to room temperature and was stirred for 1.5 hours. To this mixture was added iron(II) chloride (40 mg) and the reaction was heated at 75° C. for 15 hours. The reaction mixture was poured into water (300 mL). The resulting suspension was extracted with ethyl acetate. The combined organics were dried (magnesium sulfate), filtered and concentrated to a solid residue. This residue was purified by flash chromatography (1:1 ethyl acetate-hexane) to give 5-chloro-2-(4-fluorophenyl)pyrazolo[1,5-a]pyridine (4.2 g, 57%) as a white solid. ¹H-NMR (CDCl₃): δ 8.36 (d, 1H), 7.93 (q, 2H), 7.49 (d, 1H), 7.15 (t, 2H), 6.70 (dd, 1H), 6.69 (s, 1H); ¹⁹F-NMR (CDCl₃): δ 113.30; MS m/z 247 (M+1).

### d) 5-Chloro-2-(4-fluorophenyl)pyrazolo[1,5-a]pyridine-3-carbaldehyde

Phosphorous oxychloride (0.6 mL, 6.4 mmol) was added to N,N-dimethylformamide (10 mL) and the resulting mixture stirred at room temperature for 10 minutes. 5-Chloro-2-(4-fluorophenyl)pyrazolo[1,5-a]pyridine (1.0 g, 4.1 mmol) was added and the reaction mixture was stirred at room temperature for 12 hours. The reaction mixture was poured into ice-water and neutralized to pH 7 with aquous ammonium hydroxide. The resulting slurry was extracted with dichloromethane (3×40 mL). The combined organics were washed with brine, dried (magnesium sulfate), filtered and concentrated to give, after recrystallization from acetonitrile, 5-chloro-2-(4-fluorophenyl)pyrazolo [1,5-a]pyridine-3-carbaldehyde (0.95 g, 85%) as a white solid. ¹H-NMR (CDCl₃): δ10.07 (s, 1H), 8.49 (d, 1H), 8.44 (d, 1H), 7.78 (q, 2H), 7.22 (t, 2H), 7.07 (dd, 1H); MS m/z 275 (M+1).

### e) 1-[5-Chloro-2-(4-fluorophenyl)pyrazolo[1,5-a]pyridin-3-yl]-2-butyn-1-one

To a solution of 5-chloro-2-(4-fluorophenyl)pyrazolo[1,5-a]pyridine-3-carbaldehyde (0.93 g, 3.4 mmol) in tetrahydrofuran (20 mL) at −78° C. was added ethynylmagnesium bromide (16 mL, 0.5 M in tetrahydrofuran, 8.0 mmol). The mixture was allowed to warm to room temperature and stirred for 1 hour. Water was added to the reaction and the resulting mixture was extracted with ethyl acetate. The ethyl acetate phase was dried (magnesium sulfate), filtered and concentrated to a solid residue. This residue was dissolved in dichloromethane (50 mL) and manganese dioxide (5 g) was added. This slurry was stirred at room temperature for 2 hours. The manganese dioxide was removed by filtration and the filtrate was concentrated to a solid. This solid was purified by flash chromatography (dichloromethane) to give 1-[5-chloro-2-(4-fluorophenyl)pyrazolo[1,5-a]pyridin-3-yl]-2-butyn-1-one (0.63 g, 62% for two steps) as a white solid. ¹H-NMR (CDCl₃): δ 8.52 (d, 1H), 8.47 (d, 1H), 7.69 (q, 2H), 7.18 (t, 2H), 7.07 (dd, 1H), 3.00 (s, 1H); ¹⁹F-NMR (CDCl₃): δ 111.69; MS m/z 299 (M+1).

### f) 4-[5-Chloro-2-(4-fluorophenyl)pyrazolo[1,5-a]pyridin-3-yl]-N-cyclopentyl-2-pyrimidinamine

To a solution of 1-[5-chloro-2-(4-fluorophenyl) pyrazolo[1,5-a]pyridin-3-yl]-2-butyn-1-one (0.61 g, 2.0 mmol) in N,N-dimethylformamide was added cyclopentyl guanidine hydrochloride (0.67 g, 4.1 mmol) followed by anhydrous potassium carbonate (0.57 g, 4.1 mmol). The resulting mixture was heated at 80° C. for 12 hours. Upon cooling to room temperature, water was added. The mixture was extracted with ethyl acetate. The ethyl acetate phase was washed with brine, dried (magnesium sulfate), filtered and concentrated in vacuo. The resulting residue was purified by flash chromatography (1:1 ethyl acetate-hexane) to give, after recrystallization from acetonitrile, 4-[5-chloro-2-(4-fluorophenyl)pyrazolo[1,5-a]pyridin-3-yl]-N-cyclopentyl-2-pyrimidinamine (0.6 g, 74%) as a white solid. ¹H-NMR (CDCl₃): 68.54 (broad s, 1H), 8.40 (d, 1H), 8.04 (d, 1H), 7.60 (q, 2H), 7.16 (t, 2H), 6.88 (dd, 1H), 6.28 (d, 1H), 5.22 (d, 1H), 4.40 (m, 1H), 1.4-2.2 (m, 8H); ¹⁹F-NMR (CDCl₃): δ 112.5; MS m/z 408 (M+1).

### g) N-{4-[5-Chloro-7-(ethylsulfanyl)-2-(4-fluorophenyl)pyrazolo[1,5-a]pyridin-3-yl]-2-pyrimidinyl}-N-cyclopentylamine

To a solution of 4-[5-chloro-2-(4-fluorophenyl)pyrazolo[1,5-a]pyridin-3-yl]-N-cyclopentyl-2-pyrimidinamine (150 mg, 0.37 mmol) in anhydrous tetrahydrofuran (5 mL) at −78° C. was added n-butyllithium (0.7 mL, 1.1 mmol of 1.6 M solution in hexane). The resulting solution was stirred for 10 minutes at −78° C., followed by addition of diethyldisulfide (0.14 mL, 1.1 mmol). The reaction was stirred at −78° C. for 20 minutes and then allowed to warm to room temperature. Water and ethyl acetate were added to the reaction mixture. The phases were separated, the aqueous phase washed with ethyl acetate and the combined organic phase dried (magnesium sulfate), filtered and concentrated in vacuo. The resulting solid was purified by flash chromatography (1:1 ethyl acetate-hexane) to give, after recrystallization from ethyl acetate N-{4-[5-chloro-7-(ethylsulfanyl)-2-(4-fluorophenyl)pyrazolo[1,5-a]pyridin-3-yl]-2-pyrimidinyl}-N-cyclopentylamine (90 mg, 52%) as a solid. ¹H-NMR (CDCl₃): δ 8.42(broad s, 1H), 8.08 (d, 1H), 7.66 (q, 2H), 7.17 (t, 2H), 6.73 (d, 1H), 6.31 (d, 1H), 5.18 (d, 1H), 4.20 (m, 1H), 3.22 (q, 2H), 2.0-2.1 (m, 2H), 1.4-1.9 (m, 9H); ¹⁹F-NMR (CDCl₃): δ 112.8; MS m/z 468 (M+1).

### EXAMPLE 80

### 2-Fluoropyridin-4-ylboronic Acid

To a stirred solution of n-butyl lithium (3.2 mL, 2.5M, 8.0 mmol) in dry diethyl ether (20 mL) at −78° C. was added a solution of 2-fluoro-4-iodopyridine (1.5 g, 6.7 mmol) in dry ether (10 mL) and the reaction mixture was stirred at −78° C. for 10 minutes. Tributyl borate (2.4 mL, 2.01 g, 8.7 mmol) was added and the reaction mixture was allowed to stir to room temperature over 2 hours. Water (5 mL) was added followed by 2N aqueous sodium hydroxide solution (10 mL) to sissolve the solids. The organic phase was separated. The aqueous phase was acidified to pH3 using 6N HCl and the resulting white solid was collected by filtration and dried under vacuum to give the title compound, 0.74 g (78%). ¹H NMR (DMSO-d6) δ 8.65 (s, 2H), 8.21 (d, 1H, J=4.8 Hz), 7.59 (t, 1H, J=4.8 Hz), 7.37 (d, 1H, J=1.8 Hz).

### EXAMPLE 81

### Ethyl 3-[2-(cyclopentylamino)-4-pyrimidinyl]-2-(4-fluorophenyl)-7-methylpyrazolo[1,5-a]pyridine-6-carboxylate

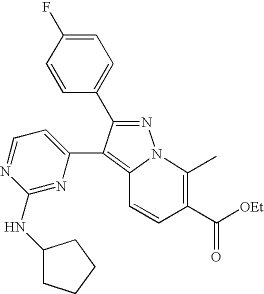

### a) 1-(4-Fluorophenyl)-2-(2-(5-trifluoromethyl)pyridyl)ethanone

To a solution of 4-fluoroacetophenone (13.8 g, 0.100 mol) and 2-chloro-5-trifluoromethylpyridine (20.0 g, 0.110 mol) in tetrahydrofuran (400 mL) was added sodium hydride (95%, 5.56 g, 0.220 mol) in several portions. The reaction was stirred at room temperature for 72 hours then carefully quenched by the addition of water (300 mL) and diethyl ether (200 mL). The organic layer was separated and extracted with 6N HCl (2×300 mL). The aqueous extracts were cooled to 0° C. and 6N NaOH was used to adjust the solution to pH12. The mixture was then extracted with diethyl ether and the combined organic extracts were dried over magnesium sulfate. The drying agent was removed by filtration and the filtrate was evaporated to dryness to afford the title compound as a tautomeric mixture, 20.9 g (73%). ¹H NMR (CDCl₃): δ 8.87(s), 8.63(s), 8.14(dd, J=5.1, 8.4 Hz), 8.00-7.83(m), 7.51(d, J=8.4 Hz), 7.22-7.12(m), 6.13(s), 4.60(s). MS (ES): 284 (M+1).

### b) 1-(4-Fluorophenyl)-2-(2-(5-trifluoromethyl)pyridyl)ethanone Oxime

To a solution of 1-(4-fluorophenyl)-2-(2-(5-trifluoromethyl)pyridyl)ethanone (80.0 g, 0.282 mol) in methanol (1 L) at room temperature was added 10% aqueous sodium hydroxide (436 mL, 1.09 mol). The resulting solution was stirred vigorously as solid hydroxylamine hydrochloride (98.0 g, 1.40 mol) was added. The mixture was heated to reflux for 2 hours, treated with decolorizing charcoal while hot, then filtered through Celite while hot. The filtrate was concentrated to one-half its original volume and then cooled to 0° C. with stirring for one hour. The resulting solids were collected by filtration, washed with water, and dried under vacuum at 50° C. overnight to provide the title compound as a light yellow powder, 73.9 g (88%). ¹H NMR (DMSO-d6): δ 11.60(s, 1H), 8.86(s, 1H), 8.14(dd, 1H, J=2.1, 8.1 Hz), 7.78(dd, 2H, J=5.7, 9.0 Hz), 7.53(d, 1H, J=8.4 Hz), 7.23(t, 2H, J=9.0 Hz), 4.40(s, 2H). MS (ES): 299 (M+1).

### c) 3-(4-Fluorophenyl)-2-(2-(5-trifluoromethyl)pyridyl)-2H-azirine

To a solution of 1-(4-fluorophenyl)-2-(2-(5-trifluoromethyl)pyridyl)ethanone oxime (25.0 g, 0.084 mol) in methylene chloride (400 mL) was added triethylamine (46.7 mL, 0.335 mol). The solution was cooled to 0° C. under a nitrogen atmosphere, and trifluoroacetic anhydride (14.1 mL, 0.100 mol) was added dropwise. The reaction was stirred for 0.5 hours then quenched with water. The organic layer was separated and dried over anhydrous magnesium sulfate. The drying agent was removed by filtration and the solvent was evaporated from the filtrate to leave an oil. The residue was loaded onto a silica gel column and eluted with 15% ethyl acetate in hexanes to give the title compound as an oil which solidified on standing, 19.4 g (82%). ¹H NMR (CDCl₃): δ 8.76(s, 1H), 7.93(dd, 2H, J=5.4, 8.7 Hz), 7.83(dd, 1H. J=2.1, 8.4 Hz), 7.27(t, 2H, J=8.7 Hz), 7.21(d, 1H, J=8.1 Hz), 3.54(s, 1H). MS (ES): 281 (M+1).

### d) 2-(4-Fluorophenyl)-6-(trifluoromethyl)pyrazolo[1,5-a]pyridine

3-(4-Fluorophenyl)-2-(2-(5-trifluoromethyl)pyridyl)-2H-azirine (40.0 g, 0.143 mol) was dissolved in 1,2,4-trichlorobenzene (400 mL) and the mixture was heated to 200° C. for 10 hours. The reaction mixture was then cooled to room temperature and poured onto a silica gel column. The column was eluted with hexanes to remove the 1,2,4-trichlorobenzene, and then with 20% diethyl ether in hexanes to elute the product. The desired fractions were combined and the solvent was evaporated under reduced pressure to leave the title compound, 28.7 g (71%). ¹H NMR (CDCl₃): δ 8.84(s, 1H), 7.98(dd, 2H, J=5.4, 8.7 Hz), 7.65(d, 1H, J=9.3 Hz), 7.28(d, 1H, J=9.3 Hz), 7.20(t, 2H, J=8.7 Hz), 6.88(s, 1H). MS (ES): 281 (M+1).

### e) 2-(4-Fluorophenyl)-6-(trifluoromethyl)pyrazolo[1,5-a]pyridine-3-carbaldehyde

To a cold (0° C.) solution of phosphorus oxychloride (8.0 mL, 86 mmol) in N,N-dimethylformamide (160 mL) was added 2-(4-fluorophenyl)-6-(trifluoromethyl)pyrazolo-[1,5-a]pyridine (11.0 g, 39.3 mmol). The reaction mixture was stirred at room temperature for 72 hours, then quenched with ice water. The solid precipitate was collected on a filter to provide 2-(4-fluorophenyl)-6-(trifluoromethyl)pyrazolo[1,5-a]pyridine-3-carbaldehyde (11.4 g, 94%) as a white solid. R_{f} 0.45 (4:1 hexanes:ethyl acetate); ¹H NMR (400 MHz, CDCl₃) δ 10.15 (s, 1H), 8.92 (s, 1H), 8.53 (d, 1H), 7.80 (m, 2H), 7.70 (d, 1H), 7.27 (t, 2H); ¹⁹F NMR (CDCl₃) δ −62.62, −110.62; MS m/z 307 (M−1).

### f) 1-[2-(4-Fluorophenyl)-6-(trifluoromethyl)pyrazolo[1,5-a]pyridin-3-yl]-2-propyn-1-ol

To a cold (−78° C.) suspension of 2-(4-fluorophenyl)-6-(trifluoromethyl)pyrazolo[1,5-a]pyridine-3-carbaldehyde (11.4 g, 37.0 mmol) in tetrahydrofuran (100 mL) was added ethynylmagnesium bromide (111 mL, 0.5 M in tetrahydrofuran, 56 mmol). The reaction mixture was warmed to room temperature and stirred for 14 hours. The reaction mixture was poured into water and adjusted to neutral pH with 1N aqueous hydrochloric acid. The aqueous mixture was extracted with ethyl acetate. The combined extracts were washed with water and brine. The organic layer was dried over magnesium sulfate. Filtration and concentration provided 1-[2-(4-fluorophenyl) 6-(trifluoromethyl)pyrazolo[1,5-a]pyridin-3-yl]-2-propyn-1-ol (11.9 g, 96%) as a tan solid. R_{f} 0.18 (4:1 hexanes:ethyl acetate); ¹H NMR (300 MHz, CDCl₃) δ 8.81 (s, 1H), 8.15 (d, 1H), 7.75 (m, 2H), 7.35 (d, 1H), 7.19 (t, 2H), 5.76 (s, 1H), 2.71 (d, 1H), 2.60 (d, 1H); MS m/z 335 (M+1).

### g) 1-[2-(4-Fluorophenyl)-6-(trifluoromethyl)pyrazolo[1,5-a]pyridin-3-yl]-2-propyn-1-one

To a cold (0° C.) solution of 1-[2-(4-fluorophenyl)-6-(trifluoromethyl)pyrazolo[1,5-a]pyridin-3-yl]-2-propyn-1-ol (5.00 g, 15.0 mmol) in chloroform (400 mL) was added manganese dioxide (130 g, 1.50 mol). The reaction mixture was stirred at 0° C. for 1.5 hours. The reaction mixture was filtered through a pad of Celite. The filtrate was concentrated in vacuo to provide 1-[2-(4-fluorophenyl)-6-(trifluoromethyl)pyrazolo-[1,5-a]pyridin-3-yl]-2-propyn-1-one (3.44 g, 69%) as a clear oil. R_{f} 0.39 (4:1 hexanes:ethyl acetate); ¹H NMR (400 MHz, CDCl₃) δ 8.90 (s, 1H), 8.61 (d, 1H), 7.72-7.69 (m, 3H), 7.17 (m, 2H), 3.06 (s, 1H); MS m/z 333 (M+1).

### h) N-Cyclopentyl-4-[2-(4-fluorophenyl)-6-(trifluoromethyl)pyrazolo[1,5-a]pyridin-3-yl]-2-pyrimidinamine

To a suspension of N-cyclopentylguanidine hydrochloride (2.20 g, 13.5 mmol) in ethanol (70 mL) was added sodium ethoxide (4.5 mL, 3 M in ethanol, 14 mmol). The mixture was stirred at room temperature for 30 minutes, then cooled to 0° C. To this mixture was added 1-[2-(4-fluorophenyl)-6-(trifluoromethyl)pyrazolo[1,5-a]pyridin-3-yl]-2-propyn-1-one (3.44 g, 10.4 mmol) portionwise. The reaction mixture was stirred at 0° C. for 30 minutes, followed by room temperature for 15 hours. The reaction mixture was diluted with water (400 mL). The solid precipitate was collected on a filter to provide N-cyclopentyl-4-[2-(4-fluorophenyl)-6-(trifluoromethyl)-pyrazolo[1,5-a]pyridin-3-yl]-2-pyrimidinamine (4.48 g, 98%) as an orange solid. ¹H NMR (400 MHz, CDCl₃) δ 8.84 (s, 1H), 8.51 (d, 1H), 8.11 (d, 1H), 7.64 (dd, 2H), 7.44 (dd, 1H), 7.17 (t, 2H), 6.33 (d, 1H), 5.17 (d, 1H), 4.34 (m, 1H), 2.15-2.06 (m, 2H), 1.84-1.52 (m, 6H); ¹⁹F NMR (CDCl₃): δ −62.70, −112.25 MS m/z 442 (M+1); mp 155-156° C.

Alternatively, N-cyclopentyl-4-[2-(4-fluorophenyl)-6-(trifluoromethyl)pyrazolo[1,5-a]pyridin-3-yl]-2-pyrimidinamine from 2-(4-Fluorophenyl)-6-(trifluoromethyl)pyrazolo[1,5-a]pyridine may be synthisized through the following steps.

### aa) 1-[2-(4-Fluorophenyl)-6-(trifluoromethyl)pyrazolo[1,5-a]pyridin-3-yl]ethanone

To a mixture of 2-(4-fluorophenyl)-6-(trifluoromethyl)pyrazolo[1,5-a]pyridine (10.30 g, 36.76 mmol) and acetic anhydride (100 mL) was added conc. sulfuric acid (10 drops) and the mixture was stirred and heated at reflux for 1 hour. The reaction mixture was cooled to room temperature and poured into ice water (300 mL). 2N Aqueous sodium hydroxide solution was added to raise the pH of the solution to about 10 and the resulting orange precipitate was collected by filtration. The solid was washed with water, air-dried, and then dried under vacuum to afford the title compound as an orange solid, 11.87 g (quant.). ¹H NMR (DMSO-d₆): δ 9.58 (s, 1H), 8.41 (d, 1H, J=9.3 Hz), 7.89 (d, 1H, J=9.5 Hz), 7.74 (m, 2H), 7.39 (m, 2H), 2.22 (s, 3H). MS (ES) 323 (M+1).

### bb) (2E)-3-(Dimethylamino)-1-[2-(4-fluorophenyl)-6-(trifluoromethyl)pyrazolo-[1,5-a]pyridin-3-yl]-2-propen-1-one

A mixture of 1-[2-(4-Fluorophenyl)-6-(trifluoromethyl)pyrazolo[1,5-a]pyridin-3-yl]ethanone (11.85 g), 36.77 mmol) and N,N-dimethylformamide dimethyl acetal (100 mL) was stirred at reflux for 17 hours. The mixture was cooled to room temperature and then to 0° C. The resulting orange precipitate was collected by filtration, washed with cold hexanes, and dried under vacuum to afford the title compound as an orange solid, 10.17 g (730/%). ¹H NMR (DMSO-d₆): δ 9.44 (s, 1H), 8.22 (d, 1H, J=9.4 Hz), 7.75 (m, 2H), 7.65 (d, 1H, J=9.5 Hz), 7.56 (d, 1H, J=12.4 Hz), 7.35 (m, 2H), 5.05 (d, 1H, J=12.3 Hz), 3.04 (s, 3H), 2.56 (s, 3H). MS (+ve ion electrospray) 377 (80), (M⁺).

### cc) N-Cyclopentyl-4-[2-(4-fluorophenyl)-6-(trifluoromethyl)pyrazolo[1,5-a]pyridin-3-yl]-2-pyrimidinamine

To a solution of (2E)-3-(dimethylamino)-1-[2-(4-fluorophenyl)-6-(trifluoromethyl)-pyrazolo[1,5-a]pyridin-3-yl]-2-propen-1-one (314 mg, 0.83 mmol)) in 1-methyl-2-pyrrolidinone (3 mL) was added N-cyclopentylguanidine hydrochloride (271 mg, 1.66 mmol) and potassium carbonate (229 mg, 1.66 mmol). The mixture was heated at 140° C. for 8 hours. Upon cooling to room temperature, ether was added followed by water. The organics were washed with brine, and the aqueous layer was extracted with ether. The combined organics were dried over magnesium sulfate, filtered and concentrated in vacuo. The residue was purified by flash chromatography on silica (4:1 hexanes-ethyl acetate) to give N-cyclopentyl-4-[2-(4-fluorophenyl)-6-(trifluoromethyl)pyrazolo[1,5-a]pyridin-3-yl]-2-pyrimidinamine (204 mg, 56%) as a white solid. ¹H NMR (CDCl₃): δ 8.84 (s, 1H), 8.51 (d, 1H), 8.11 (d, 1H), 7.64 (dd, 2H), 7.44 (dd, 1H), 7.17 (t, 2H), 6.33 (d, 1H), 5.17 (d, 1H), 4.34 (m, 1H), 2.15-2.06 (m, 2H), 1.84-1.52 (m, 6H); ¹⁹F NMR (CDCl₃): δ −62.70, −112.25; MS m/z 442 (M+1); mp 155-156° C.

### i) N-Cyclopentyl-4-[2-(4-fluorophenyl)-6-(triethoxymethyl)pyrazolo[1,5-a]pyridin-3-yl]-2-pyrimidinamine

To a dry round bottom flask was added sodium metal (1.9 g, 83 mmol). Ethanol (110 mL) was added and allowed to react with sodium at room temperature until completely dissolved. N-Cyclopentyl-4-[2-(4-fluorophenyl)-6-(trifluoromethyl)-pyrazolo[1,5-a]pyridin-3-yl]-2-pyrimidinamine (4.48 g, 10.1 mmol) was added and the reaction mixture was stirred at 60° C. for 18 hours. The reaction mixture was cooled and concentrated in vacuo to approximately one-fourth of the original volume. The resulting mixture was diluted with water and extracted with ethyl acetate. The organic layer was washed with water and brine, then dried over magnesium sulfate. Filtration and concentration provided N-cyclopentyl-4-[2-(4-fluorophenyl)-6-(triethoxymethyl)-pyrazolo[1,5-a]pyridin-3-yl]-2-pyrimidinamine (4.86 g, 92%) as an off-white solid. R_{f} 0.15 (4:1 hexanes:ethyl acetate); ¹H NMR (300 MHz, CDCl₃) δ 8.81 (s, 1H), 8.39 (d, 1H), 8.06 (d, 1H), 7.62 (m, 2H), 7.47 (d, 1H), 7.14 (t, 2H), 6.32 (d, 1H), 5.12 (d, 1H), 4.35 (m, 1H), 3.43 (q, 6H), 2.08 (m, 2H), 1.80-1.51 (m, 6H), 1.21 (t, 9H); MS m/z 520 (M+1).

### j) 4-[7-Chloro-2-(4-fluorophenyl)-6-(triethoxymethyl)pyrazolo[1,5-a]pyridin-3-yl]-N-cyclopentyl-2-pyrimidinamine

To a cold (0° C.) solution of diisopropylamine (4.1 mL, 29 mmol) in tetrahydrofuran (25 mL) was added butyllithium (17 mL, 1.6 M in hexanes, 28 mmol) dropwise. The resultant solution was stirred at 0° C. for 10 minutes then cooled to −78° C. The reaction mixture was transferred via syringe to a cold (−78° C.) solution of N-cyclopentyl-4-[2-(4-fluorophenyl)-6-(triethoxymethyl)pyrazolo[1,5-a]pyridin-3-yl]-2-pyrimidinamine (4.86 g, 9.35 mmol) in tetrahydrofuran (25 mL). The reaction mixture was stirred at −78° C. for 30 minutes. Carbon tetrachloride (3.6 mL 37 mmol) was added and the resulting mixture was warmed to room temperature and stirred for 2 hours. The reaction mixture was poured onto ice. After the ice had melted, the aqueous mixture was extracted with ethyl acetate. The organic layer was washed with water and brine, then dried over sodium sulfate. Filtration and concentration followed by flash chromatography (4:1 hexanes:ethyl acetate) provided 4-[7-chloro-2-(4-fluorophenyl)-6-(triethoxymethyl)pyrazolo[1,5-a]pyridin-3-yl]-N-cyclopentyl-2-pyrimidinamine (2.37 g, 46%) as a yellow solid. R_{f} 0.36 (4:1 hexanes:ethyl acetate); ¹H NMR (400 MHz, CDCl₃) δ 8.36 (d, 1H), 8.08 (d, 1H), 7.85 (d, 1H), 7.67 (m, 2H), 7.15 (t, 2H), 6.33 (d, 1H), 5.15 (d, 1H), 4.36 (m, 1H), 3.46 (q, 6H), 2.10 (m, 2H), 1.81-1.53 (m, 6H), 1.26 (t, 9H); MS m/554 (M+1).

### k) Ethyl 7-chloro-3-[2-(cyclopentylamino)-4-pyrimidinyl]-2-(4-fluorophenyl)-pyrazolo[1,5-a]pyridine-6-carboxylate

To a solution of 4-[7-chloro-2-(4-fluorophenyl)-6-(triethoxymethyl)pyrazolo[1,5-a]pyridin-3-yl]-N-cyclopentyl-2-pyrimidinamine (375 mg, 0.677 mmol) in acetone (8 mL) and water (2 mL) was added p-toluenesulfonic acid monohydrate (321 mg, 1.69 mmol). The reaction mixture was stirred at room temperature for 2 hours, then quenched with ice water. The reaction mixture was neutralized with saturated aqueous sodium bicarbonate solution, then concentrated in vacuo to remove the majority of the acetone. The resultant mixture was extracted with ethyl acetate. The organic layer was washed with water and brine, then dried over magnesium sulfate. Filtration and concentration, followed by flash chromatography (29:1 dichloromethane:methanol) provided ethyl 7-chloro-3-[2-(cyclopentylamino)-4-pyrimidinyl]-2-(4-fluorophenyl)pyrazolo[1,5-a]pyridine-6-carboxylate (175 mg, 540/%) as a brown solid. R_{f} 0.08 (29:1 dichloromethane:methanol); ¹H NMR (400 MHz, CDCl₃) δ 8.36 (d, 1H), 8.09 (d, 1H), 7.82 (d, 1H), 7.65 (m, 2H), 7.14 (t, 2H), 6.30 (d, 1H), 5.19 (d, 1H), 4.46 (q, 2H), 4.32 (m, 1H), 2.06 (m, 2H), 1.77-1.21 (m, 9H); MS m/z 480 (M+1).

### l) Ethyl 3-[2-(cyclopentylamino)-4-pyrimidinyl]-2-(4-fluorophenyl)-7-methylpyrazolo[1,5-a]pyridine-6-carboxylate

To a solution of ethyl 7-chloro-3-[2-(cyclopentylamino)-4-pyrimidinyl]-2-(4-fluorophenyl)pyrazolo[1,5-a]pyridine-6-carboxylate (90 mg, 0.19 mmol) in tetrahydrofuran (1 mL) was added dimethylzinc (281 μL, 2.0 M in toluene, 0.56 mmol) and tetrakis(triphenylphosphine)palladium (21 mg, 0.018 mmol). The reaction mixture was stirred at 60° C. for 16 hours. The reaction mixture was quenched with ice water then extracted with ethyl acetate. The organic layer was washed with water and brine, then dried over magnesium sulfate. Filtration and concentration, followed by flash chromatography (49:1 dichloromethane:methanol) provided ethyl 3-[2-(cyclopentylamino)-4-pyrimidinyl]-2-(4-fluorophenyl)-7-methylpyrazolo[1,5-a]pyridine-6-carboxylate (40 mg, 45%). ¹H NMR (300 MHz, CDCl₃) δ 8.34 (d, 1H), 8.11 (d, 1H), 7.91 (d, 1H), 7.70 (m, 2H), 7.19 (t, 2H), 6.36 (d, 1H), 5.33 (br, 1H), 4.47 (q, 2H), 4.38 (m, 1H), 3.26 (s, 3H), 2.12 (m, 2H), 1.83-1.43 (m, 9H); MS m/z 460 (M+1).

### EXAMPLE 82

### 3-[2-(Cyclopentylamino)-4-pyrimidinyl]-2-(4-fluorophenyl)-7-methylpyrazolo[1,5-a]pyridine-6-carboxylic Acid

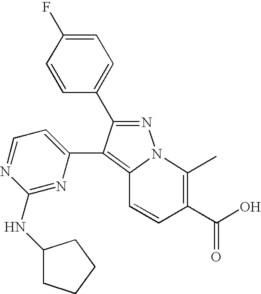

To a solution of ethyl 3-[2-(cyclopentylamino)-4-pyrimidinyl]-2-(4-fluorophenyl)-7-methylpyrazolo[1,5-a]pyridine-6-carboxylate (40 mg, 0.087 mmol) in dioxane (600 μL) was added lithium hydroxide (300 μL, 1M aqueous, 0.30 mmol). The reaction mixture was stirred at room temperature 16 hours. The reaction mixture was concentrated in vacuo to remove dioxane, then diluted with water. The aqueous mixture was acidified with 1 N aqueous hydrochloric acid. Upon standing for 72 hours, a solid precipitate had formed which was collected by filtration to provide 3-[2-(cyclopentylamino)-4-pyrimidinyl]-2-(4-fluorophenyl)-7-methylpyrazolo[1,5-a]pyridine-6-carboxylic acid (31 mg, 82%). R_{f} 0.10 (19:1 dichloromethane:methanol); MS m/z 432 (M+1).

### EXAMPLE 83

### 3-[2-(Cyclopentylamino)-4-pyrimidinyl]-N-cyclopropyl-2-(4-fluorophenyl)-7-methylpyrazolo[1,5-a]pyridine-6-carboxamide

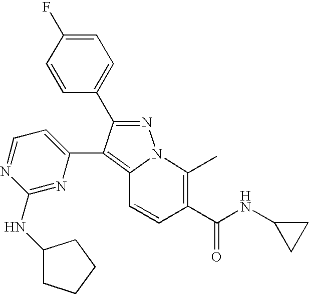

Thionyl chloride (200 μL, 2.7 mmol) was added to 3-[2-(cyclopentylamino)-4-pyrimidinyl]-2-(4-fluorophenyl)-7-methylpyrazolo[1,5-a]pyridine-6-carboxylic acid (31 mg, 0.072 mmol) which had been pre-cooled to 0° C. The reaction mixture was stirred at room temperature for 1 hour. The excess thionyl chloride was removed in vacuo. To a solution of the residue in dichloromethane (300 μL) was added cyclopropylamine (50 uL, 0.72 mmol). The reaction mixture was stirred at room temperature for 30 minutes. The resultant mixture was quenched with water and diluted with ethyl acetate. Saturated aqueous sodium bicarbonate solution was added to the biphasic mixture. The organic layer was washed with water and brine, then dried over magnesium sulfate. Filtration and concentration, followed by flash chromatography (3:2 hexanes:ethyl acetate to 2:3 hexanes:ethyl acetate) provided 3-[2-(cyclopentylamino)-4-pyrimidinyl]-N-cyclopropyl-2-(4-fluorophenyl)-7-methylpyrazolo[1,5-a]pyridine-6-carboxamide (15 mg, 44%) as a pale yellow solid. ¹H NMR (400 MHz, CDCl₃) δ 8.28 (d, 1H), 8.05 (d, 1H), 7.62 (m, 2H), 7.30 (d, 1H), 7.13 (t, 2H), 6.29 (d, 1H), 5.10 (d, 1H), 4.30 (m, 1H), 2.96 (s 3H), 2.94 (m, 1H), 2.05 (m, 2H), 1.76-1.50 (m, 6H), 0.92 (m, 2H), 0.66 (m, 2H); MS m/z 471 (M+1).

### EXAMPLE 84

### N-Butyl-4-[7-butyl-2-(4-fluorophenyl)pyrazolo[1,5-a]pyridin-3-yl]-2-pyrimidinamine

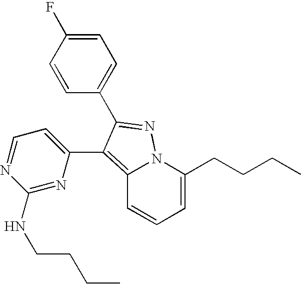

### a) 2-(6-Chloro-2-pyridinyl)-1-(4-fluorophenyl)ethanone

To a cold (0° C.) solution of 6-chloro-2-picoline (21.4 mL, 196.0 mmol) and ethyl 4-fluorobenzoate (57.5 mL, 391.2 mmol) in tetrahydrofuran (311 mL) was added lithium bis(trimethylsilyl)amide (391 mL, 1.0 M in tetrahydrofuran, 391.0 mmol) dropwise via a pressure equalizing funnel over 1 hour. Upon complete addition, the cold bath was removed and the resultant solution was heated to 45° C. for 15 hours. The mixture was cooled to room temperature and quenched by the addition of water. Ether was added and the organic layer was washed with brine. The aqueous layer was extracted with ether and the combined organics were dried over magnesium sulfate. Filtration and concentration gave a solid residue which was purified by recrystallization from ethyl acetate-hexanes to provide 2-(6-chloro-2-pyridinyl)-1-(4-fluorophenyl)ethanone (32.2 g, 66%) as a tinted off-white solid existing as a keto-enol tautomeric mixture. ¹H NMR (CDCl₃): for the keto tautomer δ 8.11 (m, 2H), 7.66 (t, 1H), 7.30-7.25 (m 2H), 7.17 (t, 2H), 4.48 (s 2H), ¹F NMR (CDCl₃) δ −104.72 (keto), −111.64 (enol); MS m/z 250 (M+1).

### b) 2-(6-Chloro-2-pyridinyl)-1-(4-fluorophenyl)ethanone Oxime

To a solution of 2-(6-chloro-2-pyridinyl)-1-(4-fluorophenyl)ethanone (74.9 g, 299.8 mmol) in methanol (900 mL) was added hydroxylamine hydrochloride (104 g, 1.49 mol) followed by sodium hydroxide (600 mL, 10% aqueous, 1.5 mol). The resultant suspension was heated to reflux for 2 hours and then cooled to room temperature. The mixture was concentrated in vacuo and the residue taken up in ether and water. The organic layer was washed with brine. The aqueous layer was extracted with ether and the combined organics were dried over magnesium sulfate. Filtration and concentration gave a solid residue which was purified by recrystallization from ethyl acetate-hexa nes to provide 2-(6-chloro-2-pyridinyl)-1-(4-fluorophenyl)ethanone oxime (67.9 g, 86%) as a white solid. ¹H NMR (CDCl₃): δ 8.69 (s, 1H), 7.71 (dd, 2H), 7.53 (t, 1H), 7.18-7.16 (m, 2H), 7.03 (t, 2H), 4.37 (s, 2H); ¹⁹F NMR (CDCl₃) δ −111.77; MS m/z 265 (M+1).

### c) 7-Chloro-2-(4-fluorophenyl)pyrazolo[1,5-a]pyridine

To a solution of 2-(6-chloro-2-pyridinyl)-1-(4-fluorophenyl)ethanone oxime (109.2 g, 414 mmol) in 1,2-dimethoxyethane. (500 mL) at 0° C. was added trifluoroacetic anhydride (59 mL, 414 mmol), keeping the temperature below 10° C. After the addition was complete, the reaction was warmed to 15° C. The solution was then cooled to 4° C. and a solution of triethylamine (116 mL, 828 mmol) in 1,2-dimethoxyethane (60 mL) was added over 0.5 hours. After warming to room temperature, the mixture was stirred for 1.5 hours. To this was added iron(II) chloride (0.52 g, 4.1 mmol) and the reaction was heated to reflux for 3 hours. The reaction was concentrated and the resulting solid was recrystallized from ethyl acetate-hexanes to give 7-chloro-2-(4-fluorophenyl)pyrazolo[1,5-a]pyridine (69.7 g, 68%) as off-white needles. ¹H NMR (CDCl₃): δ 8.03 (m, 2H), 7.54 (d, 1H), 7.16 (m, 3H), 6.93 (d, 1H), 6.91 (s, 1H); MS m/z 247 (M+1); mp 156-157° C.

### d) 7-Chloro-2-(4-fluorophenyl)pyrazolo[1,5-a]pyridine-3-carbaldehyde

N,N-Dimethylformamide (100 mL) was cooled to 0° C. and treated with phosphorous oxychloride (5.7 mL, 60.8 mmol). After the addition was complete, the mixture was warmed to room temperature and stirred for 1 hour. To this was added 7-chloro-2-(4-fluorophenyl)pyrazolo[1,5-a]pyridine (10.0 g, 40.5 mmol) and the resultant solution was stirred overnight. Water was added, followed by dichloromethane. The aqueous layer was extracted with dichloromethane. The combined organics were washed with brine, dried over magnesium sulfate, filtered and concentrated. The residue was recrystallized from diethyl ether and hexanes to give 7-chloro-2-(4-fluorophenyl)pyrazolo[1,5-a]pyridine-3-carbaldehyde (10.6 g, 95%) as a fluffy white solid. ¹H NMR (CDCl₃): δ 10.07 (s, 1H), 8.37 (d, 1H), 7.78 (m, 2H), 7.48 (t, 1H), 7.20 (m, 3H); ¹⁹F NMR (CDCl₃) 8-111.25; MS m/z 275 (M+1); Anal. Calcd for C₁₄H₈ClFN₂O: C, 61.22; H, 2.94; N, 10.20. Found: C, 61.34; H, 2.90; N, 10.15. mp 212-213° C. (decomp.).

### e) 1-[7-Chloro-2-(4-fluorophenyl)pyrazolo[1,5-a]pyridin-3-yl]-2-propyn-1-ol

In a similar manner as described in Example 81 from 7-chloro-2-(4-fluorophenyl)pyrazolo[1,5-a]pyridine-3-carbaldehyde (5.49 g, 20.0 mmol) and ethynylmagnesium bromide (100 mL 0.5 M in tetrahydrofuran, 50.0 mmol) at 0° C., recrystallized from dichloromethane, was obtained 1-[7-chloro-2-(4-fluorophenyl)pyrazolo[1,5-a]pyridin-3-yl]-2-propyn-1-ol (5.3 g, 88%) as a pale yellow crystalline solid. ¹H NMR (CDCl₃): δ 8.04 (d, 1H), 7.79 (m, 2H), 7.20 (m, 3H), 7.01 (d, 1H), 5.77 (m, 1H), 2.69 (d, 1H), 2.32 (d, 1H); MS m/z 301 (M+1).

### f) 1-[7-Chloro-2-(4-fluorophenyl)pyrazolo[1,5-a]pyridin-3-yl]-2-propyn-1-one

In a similar manner as described in Example 81, from 1-[7-chloro-2-(4-fluorophenyl)pyrazolo[1,5-a]pyridin-3-yl]-2-propyn-1-ol (5.30 g, 17.6 mmol) was obtained 1-[7-chloro-2-(4-fluorophenyl)pyrazolo[1,5-a]pyridin-3-yl]-2-propyn-1-one (4.04 g, 77%) as a yellow solid. ¹H NMR (CDCl₃): δ 8.45 (d, 1H), 7.67 (m, 2H), 7.50 (t, 1H), 7.19 (d, 1H), 7.12 (t, 2H), 2.93 (s, 1H); MS m/z 299 (M+1).

### g) N-Butyl-4-[7-chloro-2-(4-fluorophenyl)pyrazolo[1,5-a]pyridin-3-yl]-2-pyrimidinamine

In a similar manner as described in Example 81 from 1-[7-chloro-2-(4-fluorophenyl)pyrazolo[1,5-a]pyridin-3-yl]-2-propyn-1-one (0.50 g, 1.7 mmol), N-butylguanidine sulfate and sodium ethoxide (0.81 mL, 21 wt % in ethanol, 2.2 mmol) at room temperature was obtained N-butyl-4-[7-chloro-2-(4-fluorophenyl)-pyrazolo[1,5-a]pyridin-3-yl]-2-pyrimidinamine (0.39 g, 590/%) as a fluffy pale yellow solid. ¹H NMR (CDCl₃): δ 8.40 (d, 1H), 8.07 (d, 1H), 7.65 (m, 2H), 7.29 (m, 1H), 7.15 (t, 2H), 7.06 (d, 1H), 6.32 (d, 1H), 5.16 (broad s, 1H), 3.49 (q, 2H), 1.71-1.41 (m, 4H), 0.99 (t, 3H); ¹⁹F NMR (CDCl₃) δ −112.77, MS m/z 396 (M+1).

### h) N-Butyl-4-[7-butyl-2-(4-fluorophenyl)pyrazolo[1,5-a]pyridin-3-yl]-2-pyrimidinamine

To a cold (−78° C.) solution of 9-methoxy-9-borabicyclo[3.3.1]nonane (1.1 mL, 1.0 M in hexane, 1.1 mmol) in tetrahydrofuran was added n-butyllithium (696 μL, 1.6 M in hexane, 1.1 mmol) dropwise. The resultant mixture was warmed to room temperature, then potassium phosphate (371 μL 3.0 M aqueous, 1.1 mmol) was added. A solution of N-butyl-4-[7-chloro-2-(4-fluorophenyl)pyrazolo[1,5-a]pyridin-3-yl]-2-pyrimidinamine (44 mg, 0.11 mmol) and [1,1′-bis(diphenylphosphino)-ferrocene]dichloropalladium(II) (9 mg, complex with dichloromethane, 0.011 mmol) in N,N-dimethylformamide (1.5 mL) was added to the stirring borane solution. The reaction mixture was stirred 16 hours at room temperature. The resultant mixture was diluted with ethyl acetate, washed with water and brine, then dried over magnesium sulfate. Filtration and concentration, followed by flash chromatography (4:1 hexanes:ethylacetate) provided a crude residue. To a solution of the crude residue in dioxane (10 mL) was added saturated aqueous sodium acetate solution (1 mL) and 30% aqueous hygrogen peroxide (1 mL). After stirring at room temperature for 2 hours, the mixture was diluted with ethyl acetate, washed with water and brine, then dried over magnesium sulfate. Filtration and concentration, followed by flash chromatography (59:1 dichloromethane:methanol) provided N-butyl-4-[7-butyl-2-(4-fluorophenyl)pyrazolo[1,5-a]pyridin-3-yl]-2-pyrimidinamine (7 mg, 16%). ¹H NMR (400 MHz, CDCl₃) δ 8.29 (d, 1H), 8.05 (d, 1H), 7.66 (m, 2H), 7.29 (m, 1H), 7.14 (t, 2H), 6.77 (d, 1H), 6.33 (d, 1H), 5.17 (br, 1H), 3.49 (m, 2H), 3.22 (t, 2H), 1.87 (m, 2H), 1.69-1.42 (m, 6H), 1.02-0.97 (m, 6H); MS m/z 418 (M+1). To a solution of the product in ether was added 1 M HCl in ether. The precipitated solid was isolated to give the corresponding hydrochloride salt as a pale yellow solid.

### EXAMPLE 85

### N-Butyl-4-[2-(4-fluorophenyl)-7-methylpyrazolo[1,5-a]pyridin-3-yl]-2-pyrimidinamine

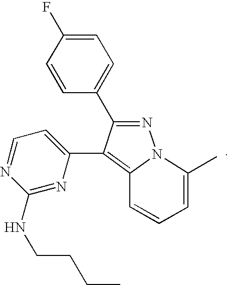

To a solution of N-butyl-4-[7-chloro-2-(4-fluorophenyl)pyrazolo[1,5-a]pyridin-3-yl]-2-pyrimidinamine (80 mg, 0.20 mmol) in tetrahydrofuran (1 mL) was added dimethylzinc (304 μL, 2.0 M in toluene, 0.60 mmol) and tetrakis(triphenylphosphine)palladium(0) (23 mg, 0.02 mmol). The reaction mixture was heated at 60° C. for 16 hours. The reaction mixture was cooled, then quenched with ice water. The resultant mixture was extracted with ethyl acetate. The organic layer was washed with water and brine, then dried over magnesium sulfate. Filtration and concentration, followed by flash chromatography (3:1 hexanes:ethyl acetate) provided N-butyl-4-[2-(4-fluorophenyl)-7-methylpyrazolo[1,5-a]pyridin-3-yl]-2-pyrimidinamine (24 mg, 32%) as a yellow solid. R_{f} 0.33 (2:1 hexanes:ethyl acetate); ¹H NMR (400 MHz, CDCl₃) δ 8.32 (d, 1H), 8.04 (d, 1H), 7.64 (m, 2H), 7.27 (m, 1H), 7.13 (t, 2H), 6.77 (d, 1H), 6.31 (d, 1H), 5.17 (br, 1H), 3.48 (m, 2H), 2.80 (s, 3H), 1.65 (m, 2H), 1.45 (m, 2H), 0.97 (t, 3H); MS m/z 376 (M+1).

### EXAMPLE 86

### N-Butyl-4-[2-(4-fluorophenyl)-7-octylpyrazolo[1,5-a]pyridin-3-yl]-2-pyrimidinamine

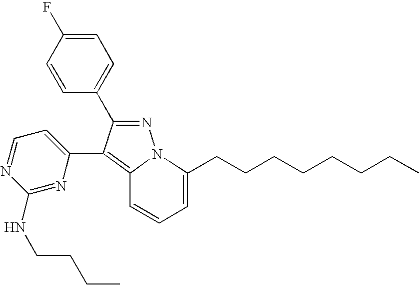

A mixture of 9-borabicyclo[3.3.1]nonane dimer (32 mg, 0.13 mmol) and tetrahydrofuran was stirred at room temperature for 2 hours. To the resultant solution was added 1-octene (38 μL 0.24 mmol) and the reaction mixture was stirred 4 hours at room temperature. Potassium phosphate (169 μL, 3 M aqueous, 0.507 mmol) was added and the reaction was stirred for 15 minutes. A solution of N-butyl-4-[7-chloro-2-(4-fluorophenyl)pyrazolo[1,5-a]pyridin-3-yl]-2-pyrimidinamine (80 mg, 0.20 mmol) and [1,1′-bis(diphenylphosphino)-ferrocene]dichloropalladium(II) (8 mg, 0.01 mmol) in N,N-dimethylformamide was added to the borane solution and stirred 18 hours. In a separate flask, 9-borabicyclo[3.3.1]nonane dimer (32 mg, 0.13 mmol) was stirred with tetrahydrofuran for 2 hours, to which 1-octene (38 μL, 0.24 mmol) was added and stirred 4 hours. Potassium phosphate (169 μL, 3 M aqueous, 0.507 mmol) was added and the solution was stirred for 15 minutes. This fresh borane solution was added to the original reaction mixture. Additional [1,1′-bis(diphenylphosphino)-ferrocene]dichloropalladium(II) (8 mg, 0.01 mmol) was added and the reaction mixture was stirred 24 hours at room temperature. The reaction mixture was diluted with ethyl acetate. The organic layer was washed with water and brine, then dried over magnesium sulfate. Filtration and concentration, followed by flash chromatography (39:1 dichloromethane:methanol) provided N-butyl-4-[2-(4-fluorophenyl)-7-octylpyrazolo[1,5-a]pyridin-3-yl]-2-pyrimidinamine (8 mg, 8%). ¹H NMR (300 MHz, CD₃OD) δ 8.80-8.55 (br, 1H), 7.84 (br, 1H), 7.71-7.65 (m, 3H), 7.35 (t, 2H), 7.17 (d, 1H), 6.55 (br, 1H), 3.32 (m, 2H), 3.26 (t, 2H), 1.90 (m, 2H), 1.75 (m, 2H), 1.54-1.25 (m, 12H), 1.03 (t, 3H), 0.89 (t, 3H); MS m/z 474 (M+1).

### EXAMPLE 87

### N-Cyclopropyl-4-[7-ethyl-2-(4-fluorophenyl)pyrazolo[1,5-a]pyridin-3-yl]-2-pyrimidinamine

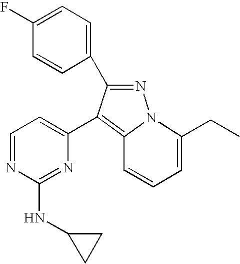

### a) 4-[7-Chloro-2-(4-fluorophenyl) pyrazolo[1,5-a]pyridin-3-yl]-N-cyclopropyl-2-pyrimidinamine

In a similar manner as described in Example 84, from 1-[7-chloro-2-(4-fluorophenyl)pyrazolo[1,5-a]pyridin-3-yl]-2-propyn-1-one (2.65 g, 8.9 mmol) and N-cyclopropylguanidine sulfate (2.27 g, 11.5 mmol) was prepared 4-[7-chloro-2-(4-fluorophenyl)pyrazolo[1,5-a]pyridin-3-yl]-N-cyclopropyl-2-pyrimidinamine (1.59 g, 47%) as a yellow solid. ¹H NMR (CDCl₃): δ 8.66 (m, 1H), 8.03 (m, 1H), 7.66 (m, 2H), 7.35 (t, 1H), 7.18 (m, 3H), 6.40 (d, 1H), 6.06 (broad, 1H), 2.90 (m, 1H), 0.91 (m, 2H), 0.70 (m, 2H); ¹⁹F NMR (CDCl₃) δ −112.22; MS m/z 380 (M+1).

### b) N-Cyclopropyl-4-[7-ethyl-2-(4-fluorophenyl)pyrazolo[1,5-a]pyridin-3-yl]-2-pyrimidinamine

In a similar manner as described in Example 85, from 4-[7-chloro-2-(4-fluorophenyl)pyrazolo[1,5-a]pyridin-3-yl]-N-cyclopropyl-2-pyrimidinamine. (100 mg, 0.26 mmol) and diethylzinc was prepared N-cyclopropyl-4-[7-ethyl-2-(4-fluorophenyl)pyrazolo[1,5-a]pyridin-3-yl]-2-pyrimidinamine (51.6 mg, 52%) as an off-white solid. ¹H NMR (CDCl₃): δ 8.51 (m, 1H), 7.99 (m, 1H), 7.63 (m, 2H), 7.35 (m, 1H), 7.16 (t, 2H), 6.82 (d, 1H), 6.37 (d, 1H), 3.25 (q, 2H), 2.87 (m, 1H), 1.45 (t, 3H), 0.88 (m, 2H), 0.67 (m, 2H); ¹⁹F NMR (CDCl₃) δ −113.32; MS m/z 374 (M+1).

### EXAMPLE 88

### Dimethyl 2-(4-fluorophenyl)-3-(4-(2-cyclopropylamino)pyrimidinyl)-7-pyrazolo[1,5-a]pyridinylcarboxamide

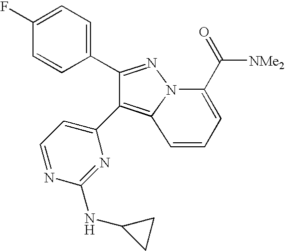

To a stirred solution of 2-(4-fluorophenyl)-7-pyrazolo[1,5-a]pyridine (5.38 g, 25 mmol) in dry tetrahydrofuran (100 mL) at −78° C. was added n-butyl lithium (2.5 M in hexanes. 12.2 mL, 30 mmol) and the mixture was stirred for 20 min. Dimethyl carbamoyl chloride (7.0 mL, 76 mmol) was added in one portion and the mixture was allowed to warm to room temperature. Diethyl ether was added followed by saturated aqueous sodium bicarbonate solution. The organic phase was separated and dried using anhydrous sodium sulfate. The drying agent was removed by filtration and the solvent was evaporated under reduced pressure. The residue was purified by silica gel chromatography to give dimethyl 2-(4-fluorophenyl)-7-pyrazolo[1,5-a]pyridinylcarboxamide as a light green solid, 6.07 g (85%). In a similar manner as described in Example 81 from dimethyl 2-(4-fluorophenyl)-7-pyrazolo[1,5-a]pyridinylcarboxamide was obtained the title compound. ¹H NMR (DMSO-d₆): δ 8.71 (d, 1H, J=8.1 Hz), 8.11 (d, 1H, J=5.1 Hz), 7.65 (m, 2H), 7.57 (dd, 1H, J=7.2, 8.7 Hz), 7.44 (d, 1H, J=2.7 Hz), 7.37 (m, 2H), 7.18 (d, 1H, J=6.3 Hz), 6.26 (d, 1H, J=5.1 Hz), 3.11 (s, 3H), 2.84 (s, 3H), 2.65 (m, 1H), 0.74 (m, 2H), 0.55 (m, 2H). MS (ES+ve): 417 (87%, M⁺).

### EXAMPLE 89

### N-Cyclopentyl-4-[2-(4-fluorophenyl)-5-morpholin-4-ylpyrazolo[1,5-a]pyridin-3-yl]pyrimidin-2-amine

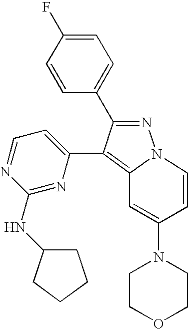

In a similar manner as described for above examples the title compound was prepared as a solid. ¹H NMR (CDCl₃): δ 8.32 (d, 1H), 8.00 (d, 1H), 7.73 (s, 1H), 7.63 (q, 2H), 7.15 (t, 2H), 6.66 (dd, 1H), 6.25 (d, 1H), 5.35 (m, 1H), 4.45 (m, 1H), 3.90 (m, 4H), 3.30 (m, 4H), 2.1-2.0 (m, 2H), 1.9-1.5 (m, 6H); ¹⁹F NMR (CDCl₃): δ −113.27; MS m/z 460 (M+1).

### EXAMPLE 90

### N¹-{4-[2-(4-Fluorophenyl)-6-trifluoromethylpyrazolo[1,5-a]pyridin-3-yl]pyrimidin-2-yl}-N³,N³-dimethylpropane-1,3-diamine

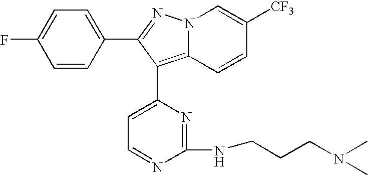

### a) 1-(4-Fluorophenyl)-2-(2-(5-trifluoromethyl)pyridyl)ethanone

To a solution of 4-fluoroacetophenone (13.8 g, 0.100 mol) and 2-chloro-5-trifluoromethylpyridine (20.0 g, 0.110 mol) in tetrahydrofuran (400 mL) was added sodium hydride (95%, 5.56 g, 0.220 mol) in several portions. The reaction was stirred at room temperature for 72 h then carefully quenched by the addition of water (300 mL) and diethyl ether (200 mL). The organic layer was separated and extracted with 6N hydrochloric acid (2×300 mL). The aqueous extracts were cooled to 0° C. and 6N sodium hydroxide was used to adjust the solution to pH12. The mixture was then extracted with diethyl ether and the combined organic extracts were dried over magnesium sulfate. The drying agent was removed by filtration and the filtrate was evaporated to dryness to afford the title compound as a tautomeric mixture, 20.9 g (73%). ¹H NMR (CDCl₃): δ 8.87 (s), 8.63 (s), 8.14 (dd, J=5.1, 8.4 Hz), 8.00-7.83 (m), 7.51 (d, J=8.4 Hz), 7.22-7.12 (m), 6.13 (s), 4.60 (s). MS (ES+ve): 284 (M+1).

### b) 1-(4-Fluorophenyl)-2-(2-(5-trifluoromethyl)pyridyl)ethanone Oxime

To a solution of 1-(4-fluorophenyl)-2-(2-(5-trifluoromethyl)pyridyl)ethanone (80.0 g, 0.282 mol) in methanol (1 L) at room temperature was added 10% aqueous sodium hydroxide (436 mL, 1.09 mol). The resulting solution was stirred vigorously as solid hydroxylamine hydrochloride (98.0 g; 1.40 mol) was added. The mixture was heated to reflux for 2 h, treated with decolorizing charcoal while hot, then filtered through Celite while hot. The filtrate was concentrated to one-half its original volume and then cooled to 0° C. with stirring for one hour. The resulting solids were collected by filtration, washed with water, and dried under vacuum at 50° C. overnight to provide the title compound as a light yellow powder, 73.9 g (88%). ¹H NMR (DMSO-d₆): δ 11.60 (s, 1H), 8.86 (s, 1H), 8.14 (dd, 1H, J=2.1, 8.1 Hz), 7.78 (dd, 2H, J=5.7, 9.0 Hz), 7.53 (d, 1H, J=8.4 Hz), 7.23 (t, 2H, J=9.0 Hz), 4.40 (s, 2H). MS (ES+ve): 299 (M+1).

### c) 3-(4-Fluorophenyl)-2-(2-(5-trifluoromethyl) pyridyl)-2H-azirine

To a solution of 1-(4-fluorophenyl)-2-(2-(5-trifluoromethyl)pyridyl)ethanone oxime (25.0 g, 0.084 mol) in methylene chloride (400 mL) was added triethylamine (46.7 mL, 0.335 mol). The solution was cooled to 0° C. under a nitrogen atmosphere, and trifluoroacetic anhydride (14.1 mL, 0.100 mol) was added dropwise. The reaction was stirred for 0.5 h then quenched with water. The organic layer was separated and dried over anhydrous magnesium sulfate. The drying agent was removed by filtration and the solvent was evaporated from the filtrate to leave an oil. The residue was loaded onto a silica gel column and eluted with 15% ethyl acetate in hexanes to give the title compound as an oil which solidified on standing, 19.4 g (82%). ¹H NMR (CDCl₃): δ 8.76 (s, 1H), 7.93 (dd, 2H, J=5.4, 8.7 Hz), 7.83 (dd, 1H, J=2.1, 8.4 Hz), 7.27 (t, 2H, J=8.7 Hz), 7.21 (d, 1H. J=8.1 Hz), 3.54 (s, 1H). MS (ES+ve): 281 (M+1).

### d) 2-(4-Fluorophenyl)-6-trifluoromethylpyrazolo[1,5-a]pyridine

3-(4-Fluorophenyl)-2-(2-(5-trifluoromethyl)pyridyl)-2H-azirine (40.0 g, 0.143 mol) was dissolved in 1,2,4-trichlorobenzene (400 mL) and the mixture was heated to 200° C. for 10 h. The reaction mixture was then cooled to room temperature and poured onto a silica gel column. The column was eluted with hexanes to remove the 1,2,4-trichlorobenzene, and then with 20% diethyl ether in hexanes to elute the product. The desired fractions were combined and the solvent was evaporated under reduced pressure to leave the title compound, 28.7 g (71%). ¹H NMR (CDCl₃): δ 8.84 (s, 1H), 7.98 (dd, 2H, J=5.4, 8.7 Hz), 7.65 (d, 1H, J=9.3 Hz), 7.28 (d, 1H, J=9.3 Hz), 7.20 (t, 2H, J=8.7 Hz), 6.88 (s, 1H). MS (ES+ve): 281 (M+1).

### e) 2-(4-Fluorophenyl)-3-acetyl-6-trifluoromethylpyrazolo[1,5-a]pyridine

To a mixture of 2-(4-fluorophenyl)-6-trifluoromethylpyrazolo[1,5-a]pyridine (10.30 g, 36.76 mmol) and acetic anhydride (100 mL) was added conc. sulfuric acid (10 drops) and the mixture was stirred and heated at reflux for 1 h. The reaction mixture was cooled to room temperature and poured into ice water (300 mL). 2N Aqueous sodium hydroxide solution was added to raise the pH of the solution to about 10 and the resulting orange precipitate was collected by filtration. The solid was washed with water, air-dried, and then dried under vacuum to afford the title compound as an orange solid, 11.87 g (quant.). ¹H NMR (DMSO-d6) δ 9.58 (s, 1H), 8.41 (d, 1H, J=9.3 Hz), 7.89 (d, 1H, J=9.5 Hz), 7.74 (m, 2H), 7.39 (m, 2H), 2.22 (s, 3H). MS 323 (M+1).

### f) 2-(4-Fluorophenyl)-3-(3-(dimethylamino)-2-propenoyl)-6-trifluoromethylpyrazolo[1,5-a]pyridine

A mixture of 2-(4-fluorophenyl)-3-acetyl-6-trifluoromethylpyrazolo[1,5-a]pyridine (11.85 g, 36.77 mmol) and N,N-dimethylformamide dimethyl acetal (100 mL) was stirred at reflux for 17 h. The mixture was cooled to room temperature and then to 0° C. The resulting orange precipitate was collected by filtration, washed with cold hexanes, and dried under vacuum to afford the title compound as an orange solid, 10.17 g (73%). ¹H NMR (DMSO-d6) δ 9.44 (s, 1H), 8.22 (d, 1H, J=9.4 Hz), 7.75 (m, 2H), 7.65 (d, 1H, J=9.5 Hz), 7.56 (d, 1H, J=12.4 Hz), 7.35 (m, 2H), 5.05 (d, 1H, J=12.3 Hz), 3.04 (s, 3H), 2.56 (s, 3H). MS 377 (M+1).

### g) N-[3-(dimethylamino)propyl]-N-[4-{2-(4-fluorophenyl)-6-(trifluoromethyl)-pyrazolo[1,5-a]pyridin-3-yl}pyrimidin-2-yl]amine

To a mixture of 2-(4-fluorophenyl)-3-(3-(dimethylamino)-2-propenoyl)-6-trifluoromethylpyrazolo[1,5-a]pyridine (2.52 g, 6.68 mmol) and N-(3-dimethylaminopropyl)guanidine (3.23 g, 2.0 equiv, 13.4 mmol) in anhydrous tetrahydrofuran (50 mL) under nitrogen was added a solution of potassium t-butoxide in t-butanol (26.7 mL, 4.0 equiv, 26.7 mmol). The mixture was stirred and heated at reflux for about 17 h and then was allowed to cool to room temperature. Water (50 mL) and diethyl ether (100 mL) were added and the organic phase was seperated. The aqueous phase was extracted with 25% tetrahydrofuran/ether. The combined organic phases were dried over anhydrous sodium sulfate and activated carbon. The drying agents were removed by filtration and the filtrate was concentrated to give the title compound as a light yellow solid 2.9 g, (95%). ¹H NMR (400 MHz, CDCl₃) δ 1.89 (m, 2H), 2.37, (s, 6H), 2.58 (br, 2H), 3.55 (dd, 2H, J=6.4, 12.4 Hz), 5.87 (br, 1H), 6.30 (d, 1H, J=5.2 Hz), 7.12 (t, 2H, J=8.4 Hz), 7.40 (d, 1H, J=9.2 Hz), 7.58 (dd, 2H, J=5.6, 8.8 Hz), 8.06 (d, 1H, J=5.2 Hz), 8.46 (d, 1H, J=9.6 Hz), 8.79 (s, 1H). MS m/z 459.50 (M+1).

### EXAMPLE 91

### 3-(2-Butoxypyridin-4-yl)-2-(4-fluorophenyl)pyrazolo[1,5-a]pyridine

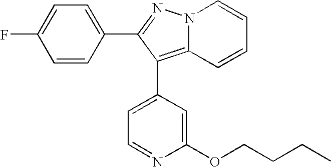

In a similar manner as described previously, using 1-butanol in place of an amine, was obtained the title compound. ¹H NMR (acetone-d₆): δ 8.70 (d, 1H, J=7.2 Hz), 8.16 (d, 1H, J=5.4 Hz), 7.76 (d, 1H, J=9.0 Hz), 7.68 (m, 2H), 7.40 (dd, 1H, J=6.9, 8.7 Hz), 7.23 (m, 2H), 7.06 (dt, 1H, J=6.9, 1.2 Hz), 6.80 (dd, 1H, J=5.4, 1.5 Hz), 6.77 (s, 1H), 4.36(t, 2H, J=6.6 Hz), 1.77(quint, 2H, J=3.9 Hz), 1.5 (sext, 2H, J=7.5 Hz), 1.0 (t, 3H, J=7.5 Hz). MS m/z: 362 (M+1).

### EXAMPLE 92

### N-Cyclopentyl-4-[2-(2,4-dimethoxyphenyl)pyrazolo[1,5-a]pyridin-3-yl]pyrimidin-2-amine

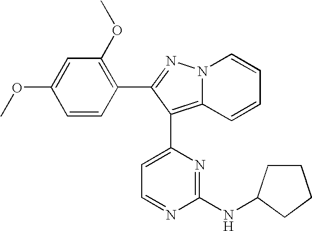

In a similar manner as described above N-cyclopentyl-4-[2-(2,4-dimethoxyphenyl)pyrazolo[1,5-a]pyridin-3-yl]pyrimidin-2-amine was obtained: ¹H NMR (CDCl3): δ 8.57 (d, 1H), 8.49 (d, 1H), 7.97 (d, 1H), 7.37 (d, 1H), 7.26 (m, 1H), 6.86 (m, 1H), 6.59 (d, 1H), 6.54 (s, 1H), 6.25 (d, 1H), 5.05 (d, 1H), 4.35 (m, 1H), 3.87 (s, 3H), 3.60 (s, 3H), 2.1-1.5 (m, 8H); MS m/z:416 (M+1).

### EXAMPLE 93

### 5-Bromo-4-[5-chloro-2-(4-fluorophenyl) pyrazolo[1,5-a]pyridin-3-yl]-N-cyclopentyl pyrimidin-2-amine

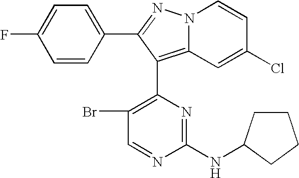

Treatment of 4-[5-chloro-2-(4-fluorophenyl)pyrazolo[1,5-a]pyridin-3-yl]-N-cyclopentylpyrimidin-2-amine (100 mg, 0.25 mmol) with N-bromosuccinimide (44 mg, 0.25 mmol) in dichloromethane (5 mL) gave after workup 110 mg (90%) of the title compound as a solid: ¹H NMR (CDCl₃): δ 8.46 (d, 1H), 8.39 (s, 1H), 7.63 (m, 3H), 7.11 (t, 2H), 6.87 (dd, 1H), 5.25 (d, 1H), 4.25 (m, 1H), 2.1-1.5 (m, 8H); ¹⁹F NMR (CDCl₃): 6-113.0; MS m/z: 487 (M+1).

### EXAMPLE 94

### N-Cyclopentyl-6-[2-(4-fluorophenyl)pyrazolo[1,5-a]pyridin-3-yl]pyrimidin-4-amine

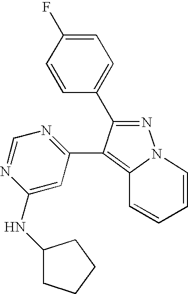

The title compound was synthesized as described previously to give: R_{f} 0.22 (2:1 hexanes:ethyl acetate); ¹H NMR (CDCl₃) δ 8.58 (s, 1H), 8.47 (d, 1H), 8.44 (d, 1H), 7.63 (m, 2H), 7.28 (t, 1H), 7.14 (t, 2H), 6.88 (t, 1H), 6.08 (s, 1H), 5.21 (br, 1H), 3.52 (m, 1H), 1.77-1.53 (m, 6H), 1.35-1.29 (m, 2H); MS m/z 374 (M+1).

### EXAMPLE 95

### N-Cyclopropyl-4-[2-(4-methoxyphenyl)-6-(trifluoromethyl)pyrazolo[1,5-a]pyridin-3-yl]pyrimidin-2-amine

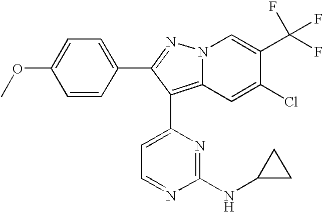

The title compound was synthesized as described previously to give: ¹H NMR: δ 8.85 (s, 1H), 8.73 (d, 1H), 8.09 (d, 1H), 7.58 (d, 2H), 7.45 (d, 1H), 7.02 (d, 2H), 6.49 (d, 1H), 5.68 (broad, 1H), 3.90 (s, 3H), 2.89 (m, 1H), 0.92 (m, 2H), 0.69 (m, 2H); ¹⁹F NMR: 6-62.66; MS m/z 425 (M+1).

### EXAMPLE 96

### N-Cyclopropyl-4-[2-(4-methoxyphenyl)-6-(triethoxymethyl)pyrazolo[1,5-a]pyridin-3-yl]pyrimidin-2-amine

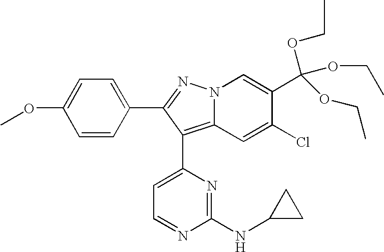

The title compound was synthesized as described previously to give: ¹H NMR: δ 8.86 (s, 1H), 8.64 (d, 1H), 8.09 (d, 1H), 7.61 (d, 2H), 7.50 (d, 1H), 7.03 (d, 2H), 6.50 (d, 1H), 5.64 (broad, 1H), 3.92 (s, 3H), 3.46 (q, 6H), 2.93 (m, 1H), 1.25 (t, 9H), 0.95 (m, 2H), 0.71 (m, 2H); MS m/z 504 (M+1).

### EXAMPLE 97

### Ethyl 3-[2-(cyclopropylamino)pyrimidin-4-yl]-2-(4-methoxyphenyl)-pyrazolo[1,5-a]pyridine-6-carboxylate

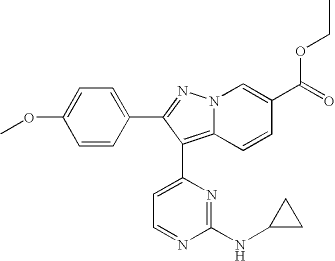

The title compound was synthesized as described previously to give: ¹H NMR: δ 9.21 (s, 1H), 8.58 (broad, 1H), 8.09 (d, 1H), 7.83 (d, 2H), 7.58 (d, 2H), 6.99 (d, 2H), 6.46 (d, 1H), 5.49 (broad, 1H), 4.43 (q, 2H), 3.88 (s, 3H), 2.87 (m, 1H), 1.42 (t, 3H), 0.89 (m, 2H), 0.65 (m, 2H); MS m/z 430 (M+1).

### EXAMPLE 98

### 3-[2-(Cyclopropylamino)pyrimidin-4-yl]-N-(2-methoxyethyl)-2-(4-methoxyphenyl)pyrazolo[1,5-a]pyridine-6-carboxamide

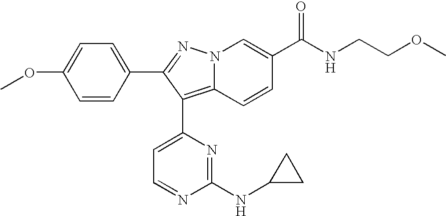

The title compound was synthesized as described previously to give: ¹H NMR: δ 9.03 (s, 1H), 8.60 (broad, 1H), 8.09 (d, 1H), 7.61-7.55 (m, 3H), 6.99 (d, 2H), 6.51 (broad, 1H), 6.46 (d, 1H), 5.42 (broad, 1H), 3.88 (s, 3H), 3.69 (q, 2H), 3.59 (q, 2H), 3.41 (s, 3H), 2.86 (m, 1H), 0.88 (m, 2H), 0.65 (m, 2H); MS m/z 459 (M+1).

### EXAMPLE 99

### 4-{5-Chloro-2-[4-(cyclopropylmethoxy)phenyl]pyrazolo[1,5-a]pyridin-3-yl}-N-cyclopropyl-2-pyrimidinamine

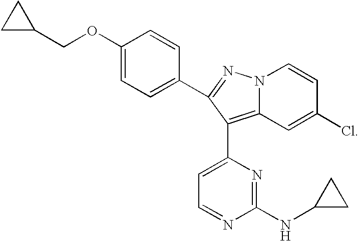

The title compound was synthesized as described previously to give: ¹H NMR CDCl₃ δ 8.80 (broad s, 1H), 8.39 (d, 1H), 8.05 (d, 1H), 7.53 (d, 2H), 6.98 (d, 2H), 6.85 (dd, 1H), 6.42 (d, 1H), 5.41 (s, 1H), 3.87 (d, 2H), 2.87 (m, 1H), 1.30 (m, 1H), 0.93 (m, 2H), 0.85 (m, 2H), 0.67 (m, 2H), 0.39 (m, 2H). MS m/432 (M+1).

### EXAMPLES 100-227

Using the techniques described above for Examples 1-99, the following additional compounds are prepared.

| Example No. | Structure |
|---|---|
| 100 | 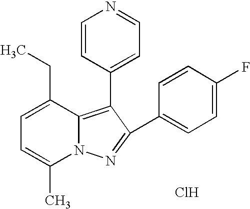 |
| 101 | 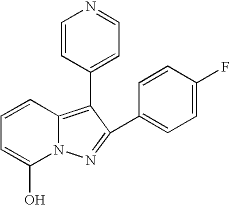 |
| 102 | 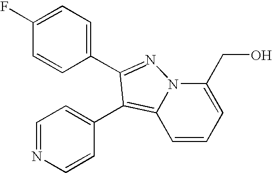 |
| 103 | 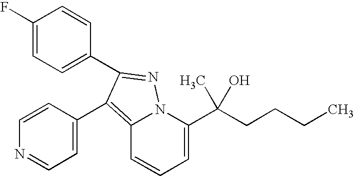 |
| 104 | 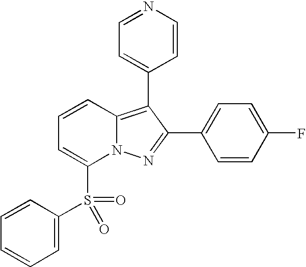 |
| 105 | 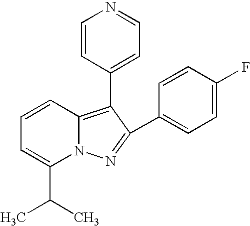 |
| 106 | 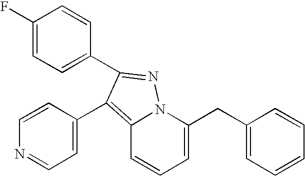 |
| 107 | 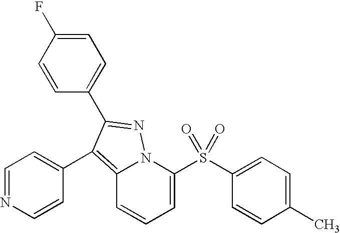 |
| 108 | 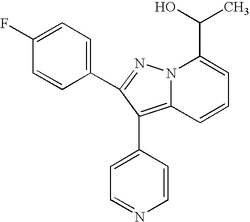 |
| 109 | 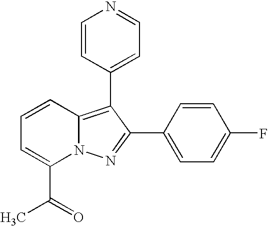 |
| 110 | 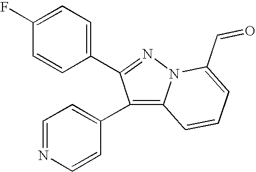 |
| 111 | 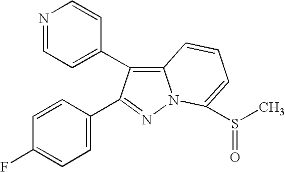 |
| 112 | 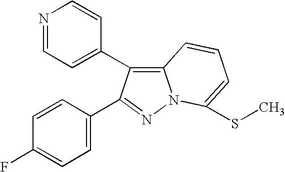 |
| 113 | 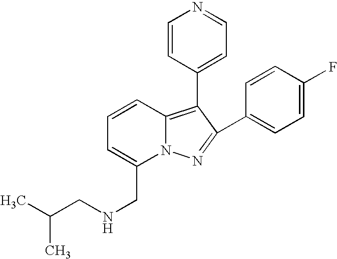 |
| 114 | 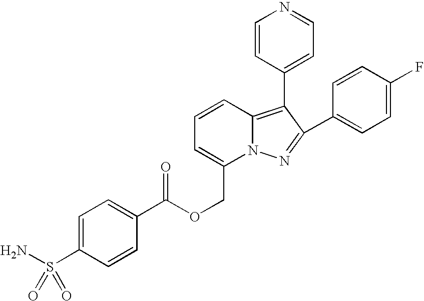 |
| 115 | 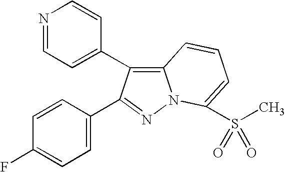 |
| 116 | 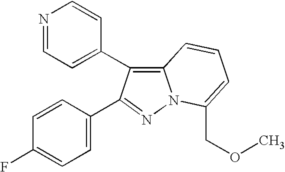 |
| 117 | 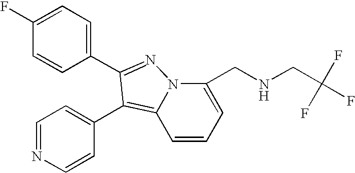 |
| 118 | 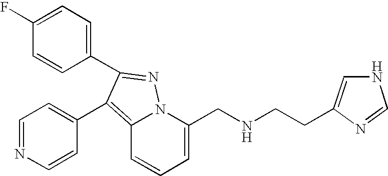 |
| 119 | 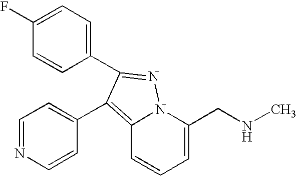 |
| 120 | 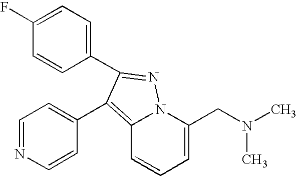 |
| 121 | 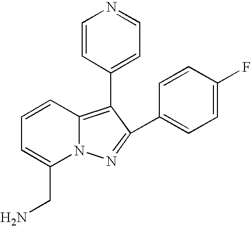 |
| 122 | 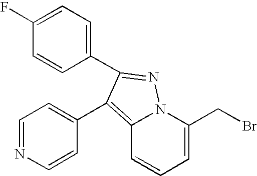 |
| 123 | 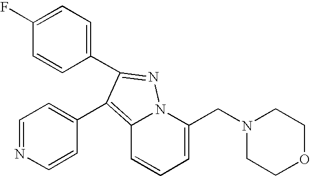 |
| 124 | 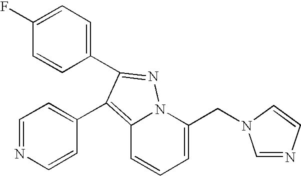 |
| 125 | 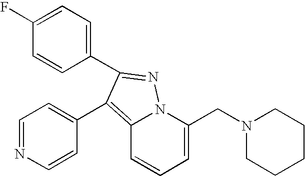 |
| 126 | 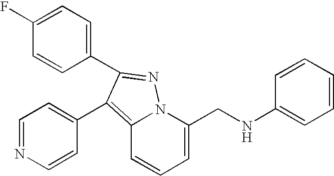 |
| 127 | 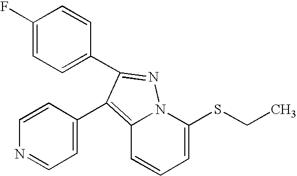 |
| 128 | 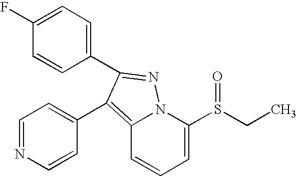 |
| 129 | 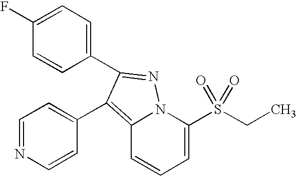 |
| 130 | 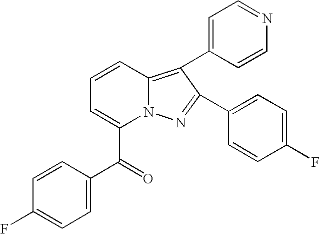 |
| 131 | 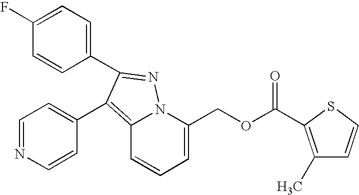 |
| 132 | 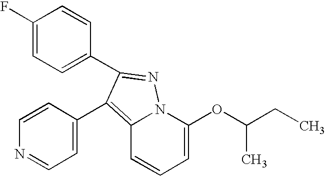 |
| 133 | 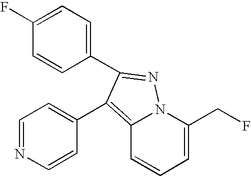 |
| 134 | 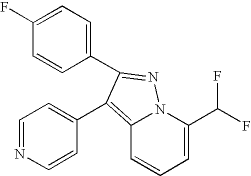 |
| 135 | 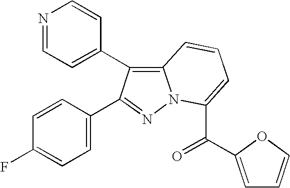 |
| 136 | 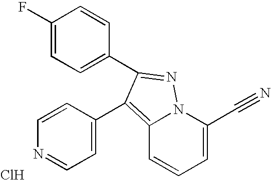 |
| 137 | 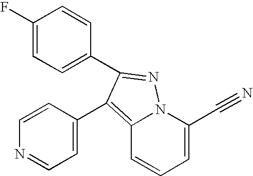 |
| 138 | 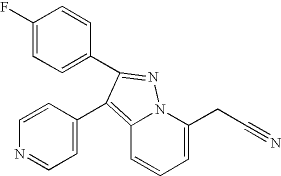 |
| 139 | 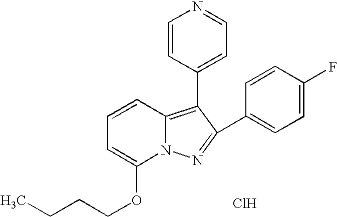 |
| 140 | 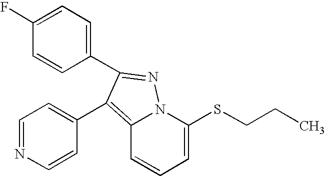 |
| 141 | 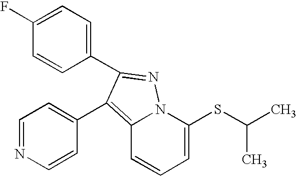 |
| 142 | 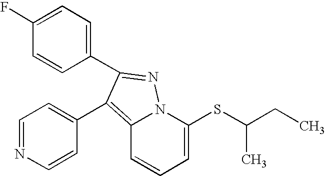 |
| 143 | 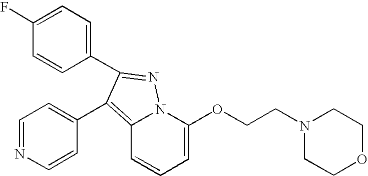 |
| 144 | 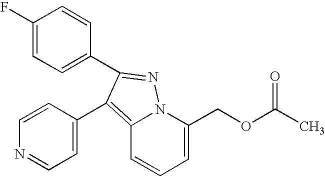 |
| 145 | 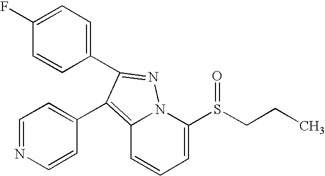 |
| 146 | 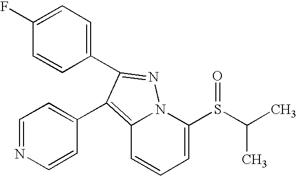 |
| 147 | 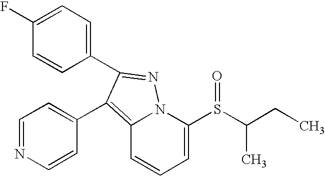 |
| 148 | 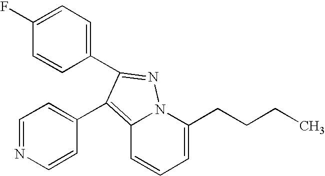 |
| 149 | 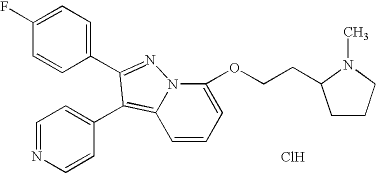 |
| 150 | 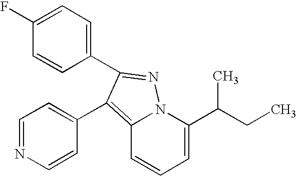 |
| 151 | 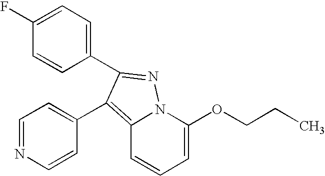 |
| 152 | 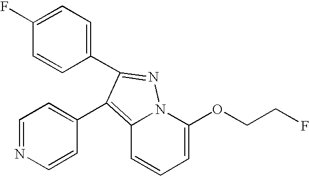 |
| 153 | 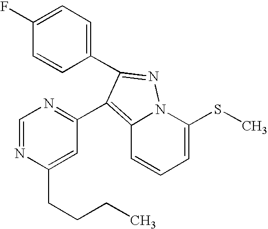 |
| 154 | 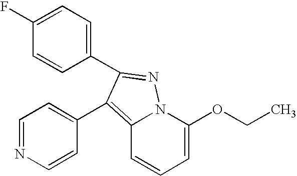 |
| 155 | 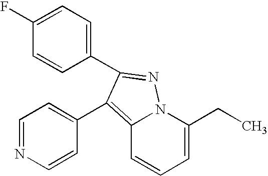 |
| 156 | 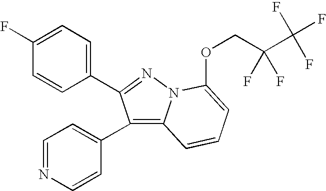 |
| 157 | 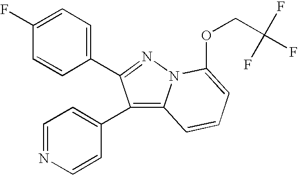 |
| 158 | 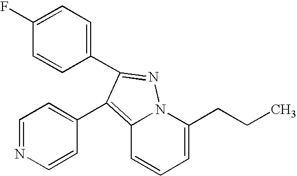 |
| 159 | 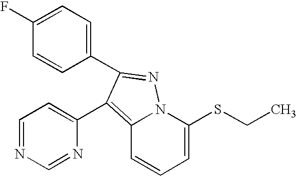 |
| 160 | 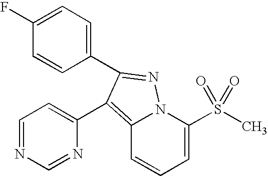 |
| 161 | 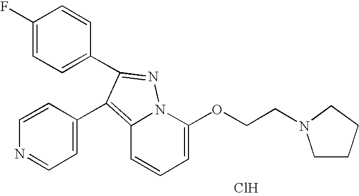 |
| 162 | 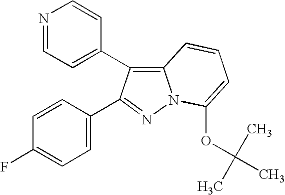 |
| 163 | 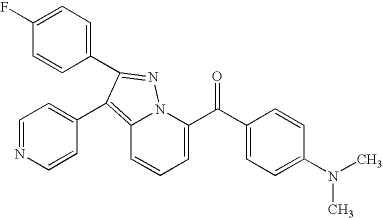 |
| 164 | 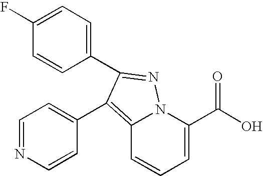 |
| 165 | 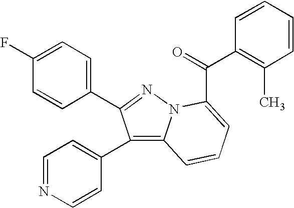 |
| 166 | 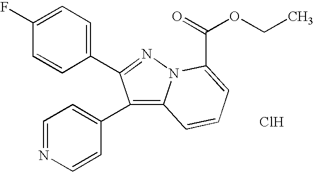 |
| 167 | 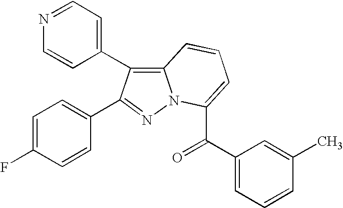 |
| 168 | 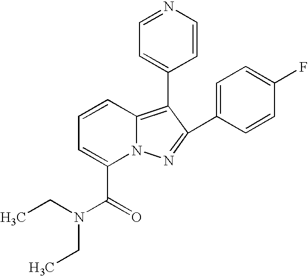 |
| 169 | 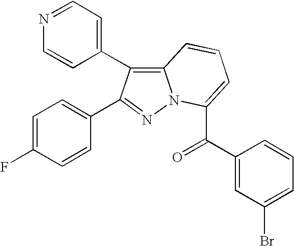 |
| 170 | 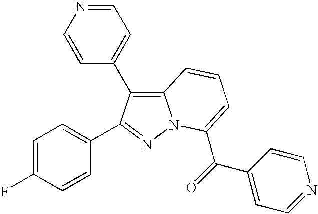 |
| 171 | 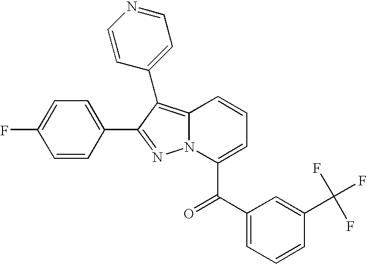 |
| 172 | 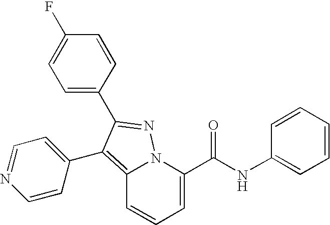 |
| 173 | 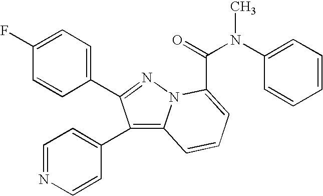 |
| 174 | 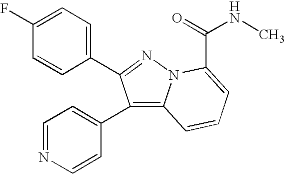 |
| 175 | 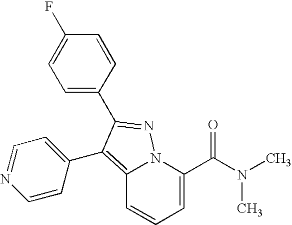 |
| 176 | 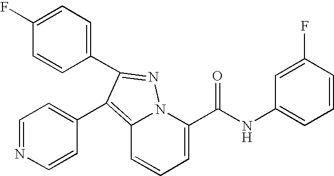 |
| 177 | 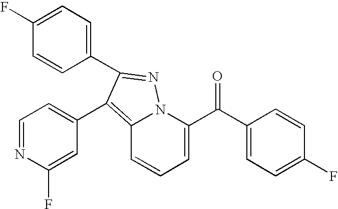 |
| 178 | 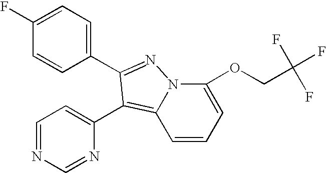 |
| 179 | 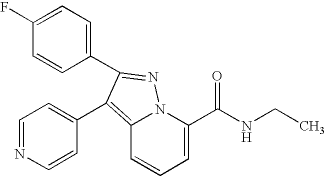 |
| 180 | 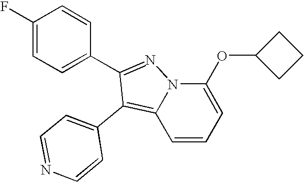 |
| 181 | 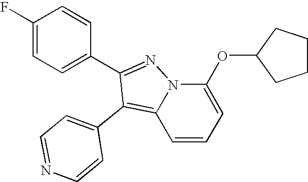 |
| 182 | 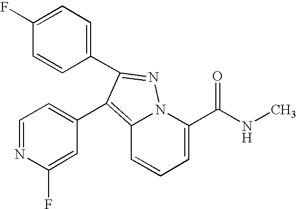 |
| 183 | 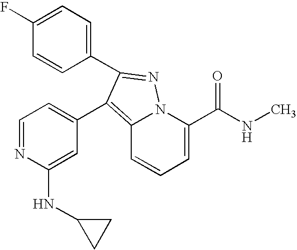 |
| 184 | 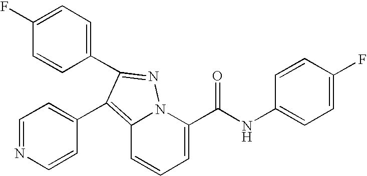 |
| 185 | 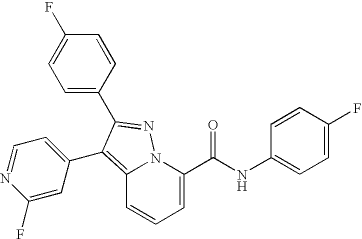 |
| 186 | 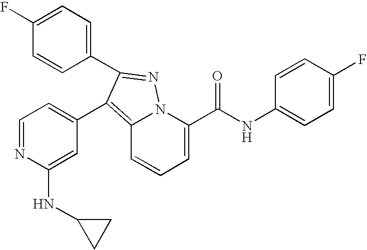 |
| 187 | 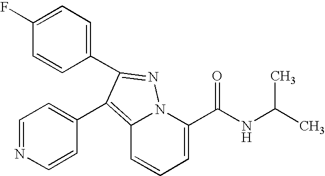 |
| 188 | 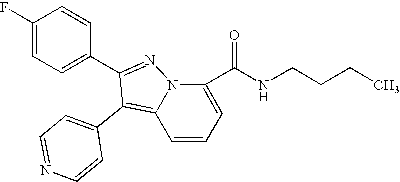 |
| 189 | 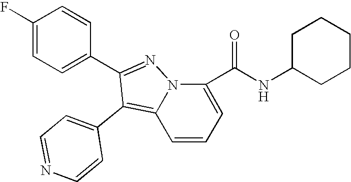 |
| 190 | 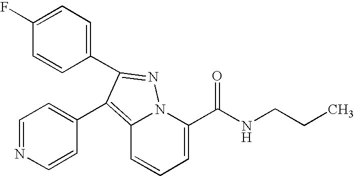 |
| 191 | 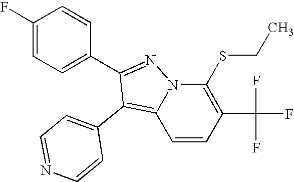 |
| 192 | 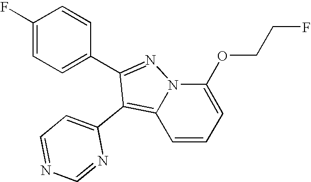 |
| 193 | 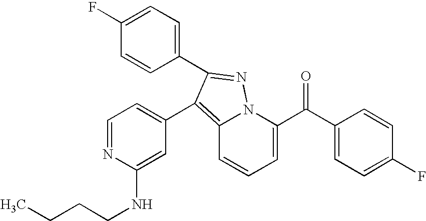 |
| 194 | 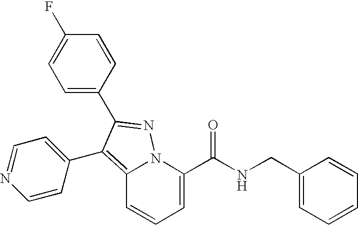 |
| 195 | 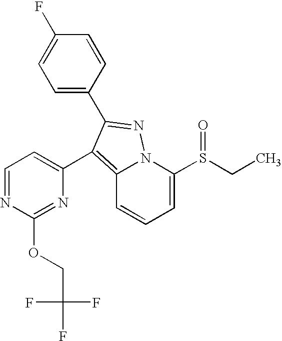 |
| 196 | 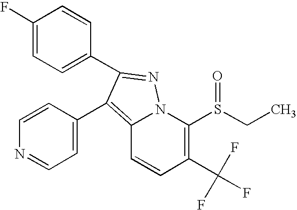 |
| 197 | 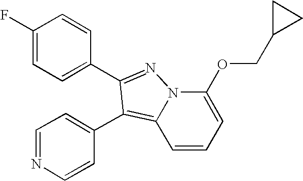 |
| 198 | 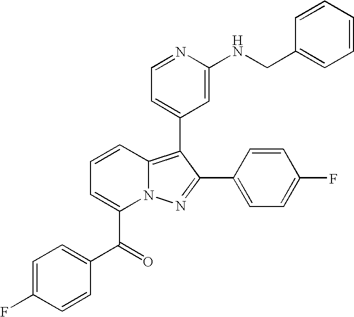 |
| 199 | 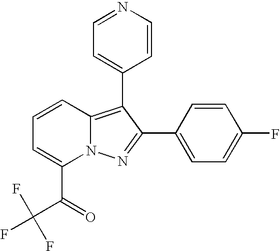 |
| 200 | 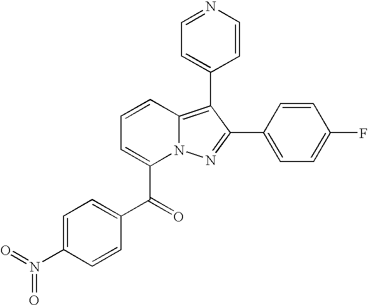 |
| 201 | 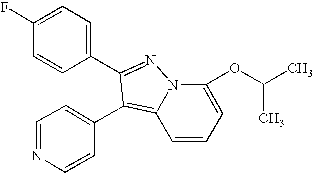 |
| 202 | 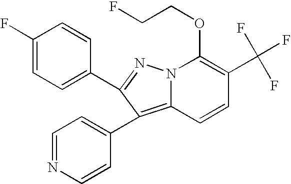 |
| 203 | 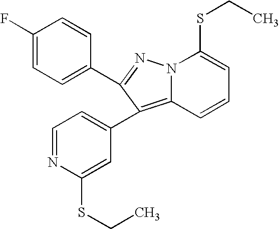 |
| 204 | 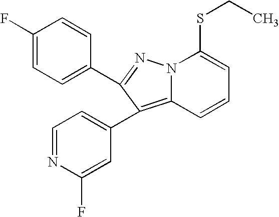 |
| 205 | 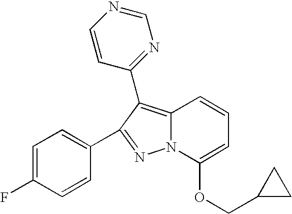 |
| 206 | 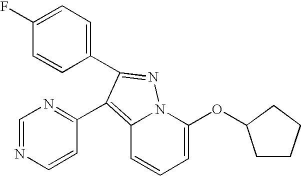 |
| 207 | 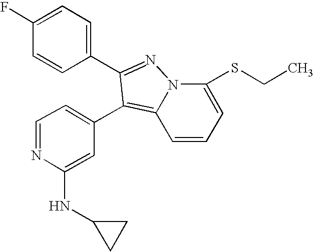 |
| 208 | 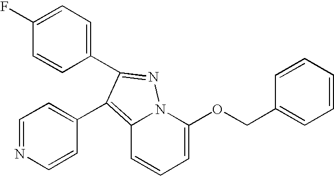 |
| 209 | 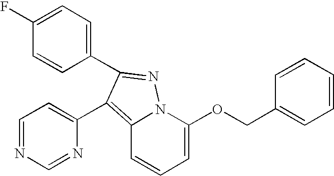 |
| 210 | 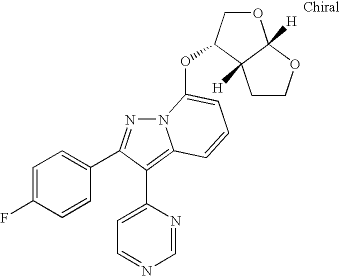 |
| 211 | 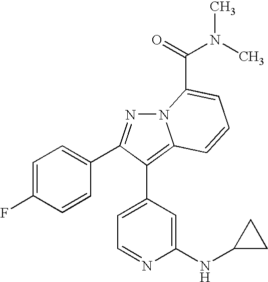 |
| 212 | 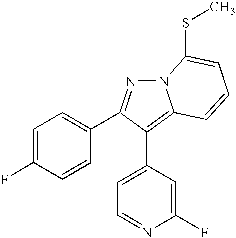 |
| 213 | 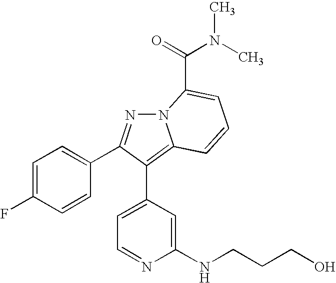 |
| 214 | 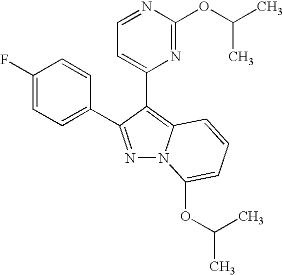 |
| 215 | 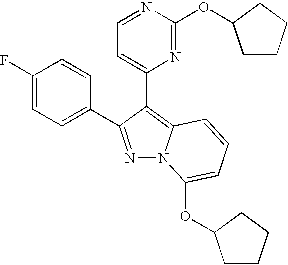 |
| 216 | 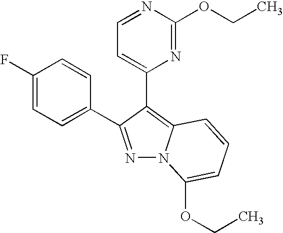 |
| 217 | 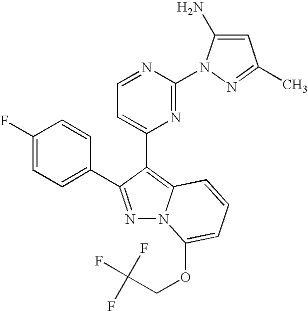 |
| 218 | 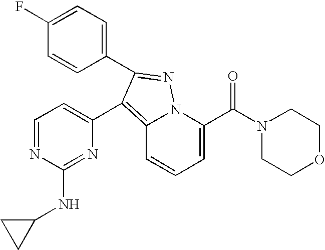 |
| 219 | 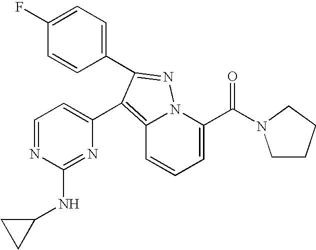 |
| 220 | 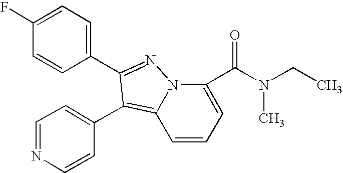 |
| 221 | 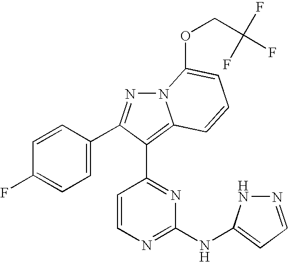 |
| 222 | 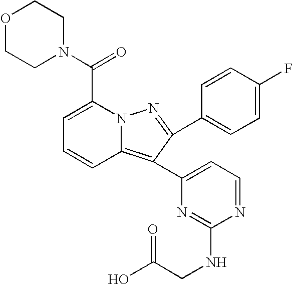 |
| 223 | 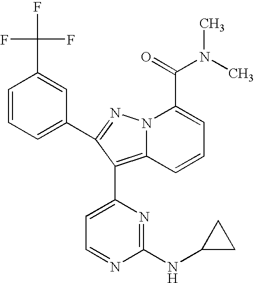 |
| 224 | 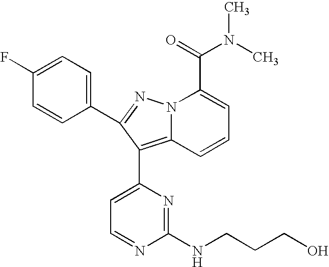 |
| 225 | 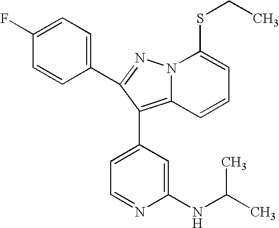 |
| 226 | 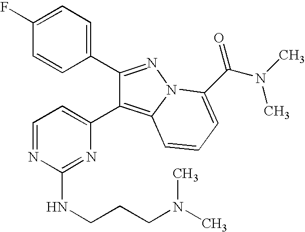 |
| 227 | 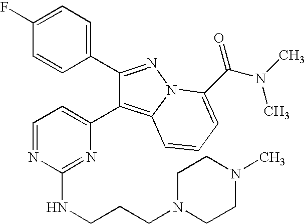 |

### EXAMPLE 228

### 4-[7-Butoxy-2-(4-methoxyphenyl)pyrazolo[1,5-a]pyridin-3-yl]-N-cyclopentyl-2-pyrimidinamine

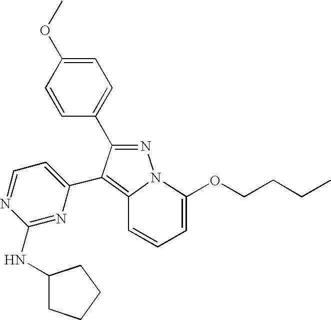

The title compound was prepared in a similar manner as described in above examples to give a yellow solid. ¹H NMR (CDCl₃): δ 8.08 (d, 1H), 8.02 (d, 1H), 7.59 (d, 2H), 7.30 (m, 1H), 6.97 (d, 2H), 6.34 (d, 1H), 6.23 (d, 1H), 5.13 (d, 1H), 4.41-4.34 (m, 3H), 3.87 (s, 3H), 2.10 (m, 2H), 1.99 (m, 2H), 1.80-1.54 (m, 8H), 1.02 (t, 3H); MS m/z 458 (M+1).

### EXAMPLE 229

### 4-[5-Chloro-2-(3-chlorophenyl)-7-(methylsulfanyl)pyrazolo[1,5-a]pyridin-3-yl]-N-cyclopentyl-2-pyrimidinamine

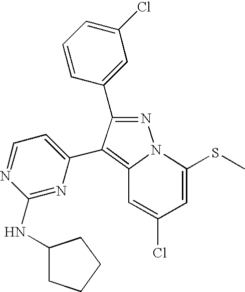

The title compound was prepared in a similar manner as described in above examples to give a yellow solid. R_{f} 0.23 (4:1 hexanes:ethyl acetate); ¹H NMR (CDCl₃) δ 8.37 (s, 1H), 8.02 (d, 1H), 7.70 (s, 1H), 7.50 (d, 1H), 7.45-7.33 (m, 2H), 6.61 (s, 1H), 6.29 (d, 1H), 5.20 (d, 1H), 4.36 (m, 1H), 2.65 (s, 3H), 2.15 (m, 2H), 1.84-1.52 (m, 6H); MS m/z 470 (M+1).

### EXAMPLE 230

### N-cyclopentyl-6-[2-(4-fluorophenyl)-7-(methylthio)pyrazolo[1,5-a]pyridin-3-yl]pyrimidin-4-amine

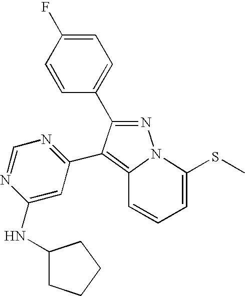

The title compound was prepared in a similar manner as described in above examples to give a peach colored solid. ¹H NMR (CDCl₃) δ 8.60 (s, 1H), 8.26 (d, 1H), 7.86 (m, 2H), 7.32 (t, 1H), 7.15 (t, 2H), 6.70 (d, 1H), 6.08 (s, 1H), 4.95 (br, 1H), 3.58 (br, 1H), 2.65 (s, 3H), 1.85-1.50 (m, 6H), 1.38-1.22 (m, 2H); MS m/z 420 (M+1).

### EXAMPLE 231

### N-cyclopentyl-4-[2-(4-fluorophenyl)-7-(methylthio)-5-morpholin-4-ylpyrazolo[1,5-a]pyridin-3-yl]pyrimidin-2-amine

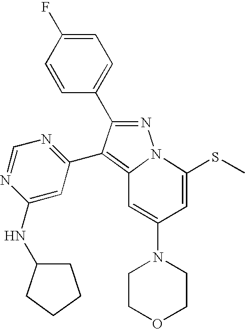

In a similar manner as described for above examples the title compound was prepared as a solid. ¹H NMR (CDCl₃): δ 8.01 (d, 1H), 7.64 (m, 3H), 7.15 (t, 2H), 6.45 (d, 1H), 6.26 (d, 1H), 5.17 (d, 1H), 4.46 (m, 1H), 3.93 (m, 4H), 3.33 (m, 4H), 2.66 (s, 3H), 2.1-1.5 (m, 8H); ¹⁹F NMR (CDCl₃): δ −113.5; MS m/z 505 (M+1).

### EXAMPLE 232

### N-Cyclopentyl-4-[2-(4-fluorophenyl)-7-(isopropylthio)-5-morpholin-4-ylpyrazolo[1,5-a]pyridin-3-yl]pyrimidin-2-amine

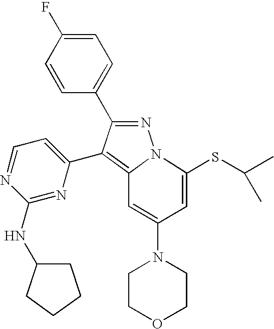

In a similar manner as described for above examples the title compound was prepared as a solid. ¹H NMR (CDCl₃): δ 8.01 (d, 1H), 7.66 (m, 3H), 7.15 (t, 2H), 6.71 (d, 1H), 6.27 (d, 1H), 5.21 (d, 1H), 4.44 (m, 1H), 3.93 (m, 4H), 3.31 (m, 4H), 2.1-1.5 (m, 9H), 1.44 (d, 6H); ¹⁹F NMR (CDCl₃): δ −113.6; MS m/z 534 (M+1).

### EXAMPLE 233

### Biological Activity

In the following example, “MEM” means Minimal Essential Media; “FBS” means Fetal Bovine Serum; “NP40” and “Igepal” are detergents; “MOI” means Multiplicity of Infection; “NaOH” means sodium hydroxide; “MgCl₂” means magnesium chloride; “dATP” means deoxyadenosine 5′ triphosphate; “dUTP” means deoxyuridine 5′ triphosphate; “dCTP” means dexoxycytidine 5′ triphosphate; “dGTP” means deoxyguanosine 5′ triphosphate; “GuSCN” means Guanidinium thiocyanate; “EDTA” means ethylenediamine tetraacetic acid; “TE” means Tris-EDTA; “SCC” means sodium chloride/sodium citrate; “APE” means a solution of ammonia acetate, ammonia phosphate, EDTA; “PBS” means phosphate buffered saline; and “HRP” means horseradish peroxidase.

a) Tissue Culture and HSV Infection.

Vero 76 cells were maintained in MEM with Earle's salts, L-glutamine, 8% FBS (Hyclone, A-1111-L) and 100 units/mL Penicillin-100 μg/mL Streptomycin. For assay conditions, FBS was reduced to 2%. Cells are seeded into 96-well tissue culture plates at a density of 5×10⁴ cells/well after being incubated for 45 min at 37° C. in the presence of HSV-1 or HSV-2 (MOI=0.001). Test compounds are added to the wells and the plates are incubated at 37° C. for 40-48 hours. Cell lysates are prepared as follows: media was removed and replaced with 150 μL/well 0.2 N NaOH with 1% Igepal CA 630 or NP-40. Plates were incubated up to 14 days at room temperature in a humidified chamber to prevent evaporation.

(b) Preparation of Detection DNA.

For the detection probe, a gel-purified, digoxigenin-labeled, 710-bp PCR fragment of the HSV UL-15 sequence was utilized. PCR conditions included 0.5 μM primers, 180 μM dTTP, 20 μM dUTP-digoxigenin (Boehringer Mannheim 1558706), 200 μM each of dATP, dCTP, and dGTP, 1×PCR Buffer II (Perkin Elmer), 2.5 mM MgCl₂, 0.025 units/μL of AmpliTaq Gold polymerase (Perkin Elmer), and 5 ng of gel-purified HSV DNA per 100 μL Extension conditions were 10 min at 95° C., followed by 30 cycles of 95° C. for 1 min, 55° C. for 30 sec, and 72° C. for 2 min. The amplification was completed with a 10-min incubation at 72° C. Primers were selected to amplify a 728 bp probe spanning a section of the HSV1 UL15 open reading frame (nucleotides 249-977). Single-stranded transcripts were purified with Promega M13 Wizard kits. The final product was mixed 1:1 with a mixture of 6 M GuSCN, 100 mM EDTA and 200 μg/mL herring sperm DNA and stored at 4° C.

(c) Preparation of Capture Plates.

The capture DNA plasmid (HSV UL13 region in pUC) was linearized by cutting with Xba I, denatured for 15 min at 95° C. and diluted immediately into Reacti-Bind DNA Coating Solution (Pierce, 17250, diluted 1:1 with TE buffer, pH 8) at 1 ng/μL. 75 μL/well were added to Corning (#3922 or 9690) white 96-well plates and incubated at room temperature for at least 4 hrs before washing twice with 300 μL/well 0.2×SSC/0.05% Tween-20 (SSC/T buffer). The plates were then incubated overnight at room temperature with 150 μL/well 0.2 N NaOH, 1% IGEPAL and 10 μg/mL herring sperm DNA.

(d) Hybridization

Twenty-seven (27) μL of cell lysate was combined with 45 μL of hybridization solution (final concentration: 3M GuSCN, 50 mM EDTA, 100 μg/ml salmon sperm DNA, 5×Denhardt's solution, 0.25×APE, and 5 ng of the digoxigenin-labeled detection probe). APE is 1.5 M NH₄-acetate, 0.15 M NH₄H₂ phosphate, and 5 mM EDTA adjusted to pH 6.0. Mineral oil (50 μL) was added to prevent evaporation. The hybridization plates were incubated at 95° C. for 10 minutes to denature the DNA, then incubated at 42° C. overnight. The wells were washed 6× with 300 μL/well SSC/T buffer then incubated with 75 μL/well anti-digoxigenin-HRP-conjugated antibody (Boehringer Mannheim 1207733, 1:5000 in TE) for 30 min at room temperature. The wells were washed 6× with 300 μL/well with PBS/0.05% Tween-20 before 75 μL/well SuperSignal LBA substrate (Pierce) was added. The plates were incubated at room temperature for 30 minutes and chemiluminescence was measured in a Wallac Victor reader.

e) Results.

The following results were obtained for HSV-1.

| Example No. | IC₅₀ (μM) |
|---|---|
| 1 | 0.7 |
| 2 | 1.6 |
| 3 | 1.5 |
| 4 | 20 |
| 5 | 5 |
| 6 | 4.2 |
| 7 | 6.2 |
| 8 | 2.8 |
| 9 | 2.4 |
| 11 | 4.4 |
| 12 | 3.9 |
| 13 | 1.2 |
| 14 | 2 |
| 15 | 4 |
| 16 | >20 |
| 17 | 1.5 |
| 18 | 5.4 |
| 19 | >40 |
| 20 | 16 |
| 21 | 0.8 |
| 22 | 4.3 |
| 23 | 1 |
| 24 | 0.2 |
| 25 | 5.5 |
| 26 | 0.15 |
| 27 | 0.5 |
| 28 | 0.73 |
| 29 | 10 |
| 30 | 4 |
| 31 | 1.7 |
| 32 | 10 |
| 33 | 2.5 |
| 34 | 2 |
| 35 | 4.5 |
| 36 | 5 |
| 37 | 1.4 |
| 38 | 3.8 |
| 39 | 1.7 |
| 40 | 5.3 |
| 41 | 21.6 |
| 42 | 5 |
| 43 | 2.4 |
| 44 | 6.3 |
| 45 | 2.6 |
| 47 | 1 |
| 48 | 0.9 |
| 49 | 2.9 |
| 57 | 2.5 |
| 59 | 10 |
| 60 | 5 |
| 62 | 2 |
| 64 | 0.9 |
| 66 | 25 |
| 67 | 15 |
| 68 | 15 |
| 83 | 1 |
| 84 | 0.6 |
| 85 | 1 |
| 86 | 6 |
| 87 | 3 |
| 88 | 15 |
| 89 | 3.6 |
| 90 | 1.9 |
| 91 | 11 |
| 92 | 20 |
| 93 | 2.6 |
| 95 | 27 |
| 96 | 5.5 |
| 97 | 16 |
| 98 | 1.1 |
| 228 | 0.28 |
| 229 | 1.0 |
| 230 | 1.3 |
| 231 | 0.5 |
| 232 | 0.9 |

The results demonstrate that the compounds of the present invention are useful for the treatment and prophylaxis of herpes viral infections.

1. A method for the prophylaxis or treatment of a herpes viral infection in an animal, said method comprising administering to the animal a therapeutically effective amount of a compound of formula (I): 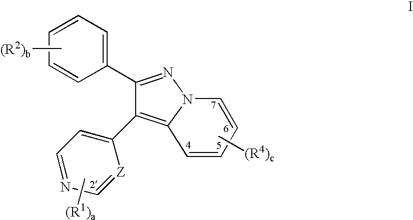
wherein:
Z is CH or N;
a is 1 or 2;
b is 1, 2 or 3;
c is 1, 2 or 3;
each R¹ is independently selected from group consisting of substituents of the formula
—(X)_{d}—(CH₂)ₑ—R⁵
wherein
d is 0 or 1;
e is 0 to 6;
X is selected from the group consisting of O, NR⁶ and S(O)_{f} where f is 0, 1 or 2;
R⁵ is selected from the group consisting of H, halo, C₁₋₆alkyl, C₂₋₆alkenyl, C₂₋₆alkynyl, cycloalkyl, heterocyclyl, aryl, heteroaryl, hydroxyl, cyano, nitro, trihalomethyl, NR⁷R⁸, C₆H₄NR⁷R⁸, C₆H₄(CH₂)NR⁷R⁸, C(O)R⁷, C(O)NR⁷R⁸, OC(O)R⁷, OC(O)NR⁷R⁸, CO₂R⁷, OCO₂R⁷, SO₂R⁷, SO₂NR⁷R⁸, C(═NR⁷)NR⁷R⁸, N(R⁷) [(C═NR⁷)NR⁷R⁸], NHC(O)R⁷ and N(C₁₋₃alkyl)C(O)R⁷;
each R² is independently selected from the group consisting of H, cyano, halo, trihalomethyl, OC₁₋₆alkyl, C₁₋₆alkyl, C₂₋₆alkenyl, C₂₋₆alkynyl, S(O)_{g}C₁₋₆alkyl where g is 0, 1 or 2, NC₁₋₆alkyl(C₁₋₆alkyl), hydroxyl and nitro;
each R⁴ is independently selected from the group consisting of substituents of the formula
—(Y)_{d}—(CH₂)ₑ—R³
wherein
d is 0 or 1;
e is 0 to 6;
Y is O or S(O)_{f} where f is 0, 1 or 2;
R³ is selected from the group consisting of H, halo, C₁₋₆alkyl, C₂₋₆alkenyl, C₂₋₆alkynyl, cycloalkyl, heterocyclyl, aryl, heteroaryl, hydroxyl, cyano, nitro, trihalomethyl, phthalamido, C₆H₄NR⁷R⁸, C₆H₄(CH₂)NR⁷R⁸, C(O)R⁷, C(O)NR⁷RB, OC(O)R⁷, OC(O)NR⁷R⁸, CO₂R⁷, OCO₂R⁷, SO₂R⁷, SO₂NR⁷R⁸ and C(═NR⁷)NR⁷R⁸;
R⁶ is selected from the group consisting of H, C₁₋₆alkyl, C₂₋₆alkenyl, heteroaryl, cycloalkyl, and heterocyclyl;
R⁷ and R⁸ are each independently selected from the group consisting of H, C₁₋₈alkyl, C₂₋₆alkenyl, SO₂C₁₋₆alkyl, (CH₂)ₘ-cycloalkyl, (CH₂)ₘ-aryl, (CH₂)ₘ-heterocyclyl and (CH₂)ₘ-heteroaryl, wherein m is 0, 1 or 2,
or R⁷ and R⁸ together with the nitrogen atom to which they are bound, form a heterocyclyl group; and
wherein any of said alkyl, alkenyl and alkynyl groups may be optionally substituted with up to three members selected from the group consisting of halo, hydroxyl, oxo, cyano, NR⁷R⁸, C₁₋₆alkyl, OC₁₋₆alkyl, S(O)C₁₋₆alkyl, S(O)₂C₁₋₆alkyl and SO₂NR⁷R⁸; and
wherein any of said cycloalkyl, heterocyclyl, aryl and heteroaryl groups may be optionally substituted with up to three substituents selected from the group consisting of C₁₋₆alkyl, C₁₋₆alkoxy, C₁₋₆alkylsulfenyl, C₁₋₆alkylsulfinyl, C₁₋₆alkylsulfonyl, hydroxy, oxo, mercapto, nitro, cyano, halogen, C₁₋₆perfluoroalkyl, amino optionally substituted by C₁₋₆alkyl, carbamoyl optionally substituted by C₁₋₆alkyl, NR⁷R⁸, carboxy and aminosulfonyl optionally substituted by C₁₋₆alkyl;
wherein when (R¹)ₐ is located at the 2′ position, (R¹)ₐ is not NR⁶-aryl, NR⁶—C₆H₄NR⁷R⁸, NR⁶—C₆H₄(CH₂)NR⁷R⁸, NR⁷R⁸ where R⁷ or R⁸ is (CH₂)ₘ-aryl and m is O, or N-(aryl)[(C═NR⁷)NR⁷R⁸]; and
wherein when R⁴ is at the C-7 position, R⁴ is not halo, heterocyclyl, aryl, heteroaryl, phthalamido, C₆H₄NR⁷R⁸ or C₆H₄(CH₂)NR⁷R⁸;
or a pharmaceutically acceptable salt, solvate or physiologically functional derivative thereof.

2. The method according to claim 1 wherein said herpes viral infection is selected from the group consisting of herpes simplex virus 1, herpes simplex virus 2, cytomegalovirus, Epstein Barr virus, varicella zoster virus, human herpes virus 6, human herpes virus 7, and human herpes virus 8.

3. A method for the prophylaxis or treatment of a condition or disease associated with a herpes viral infection in an animal, comprising administering to the animal a therapeutically effective amount of a compound of formula (I): 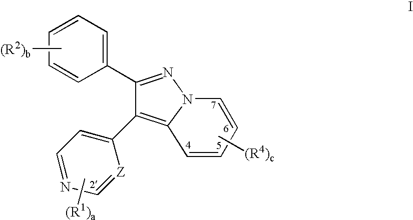
wherein:
Z is CH or N;
a is 1 or 2;
b is 1, 2 or 3;
c is 1, 2 or 3;
each R¹ is independently selected from the group consisting of substituents of the formula
—(X)_{d}—(CH₂)ₑ—R⁵
wherein:
d is 0 or 1;
e is 0 to 6;
X is selected from the group consisting of O, NR⁶ and S(O)_{f} where f is 0, 1 or 2;
R⁵ is selected from the group consisting of H, halo, C₁₋₆alkyl, C₂₋₆alkenyl, C₂₋₆alkynyl, cycloalkyl, heterocyclyl, aryl, heteroaryl, hydroxyl, cyano, nitro, trihalomethyl, NR⁷R⁸, C₆H₄NR⁷R⁸, C₆H₄(CH₂)NR⁷R⁸, C(O)R⁷, C(O)NR⁷R⁸, OC(O)R⁷, OC(O)NR⁷R⁸, CO₂R⁷, OCO₂R⁷, SO₂R⁷, SO₂NR⁷R⁸, C(═NR⁷)NR⁷R⁸, N(R⁷)[(C═NR⁷)NR⁷R⁸], NHC(O)R⁷ and N(C₁₋₃alkyl)C(O)R⁷;
each R² is independently selected from the group consisting of H, cyano, halo, trihalomethyl, OC₁₋₆alkyl, C₁₋₆alkyl, C₂₋₆alkenyl, C₂₋₆alkynyl, S(O)_{g}C₁₋₆alkyl where g is 0, 1 or 2, NC₁₋₆alkyl(C₁₋₆alkyl), hydroxyl and nitro;
each R⁴ is independently selected from the group consisting of substituents of the formula
—(Y)_{d}—(CH₂)ₑ—R³
wherein:
d is 0 or 1;
e is 0 to 6;
Y is O or S(O)_{f} where f is 0, 1 or 2;
R³ is selected from the group consisting of H, halo, C₁₋₆alkyl, C₂₋₆alkenyl, C₂₋₆alkynyl, cycloalkyl, heterocyclyl, aryl, heteroaryl, hydroxyl, cyano, nitro, trihalomethyl, phthalamido, C₆H₄NR R⁸, C₆H₄(CH₂)NR⁷R⁸, C(O)R⁷, C(O)NR⁷R⁸, OC(O)R⁷, OC(O)NR R⁸, CO₂R⁷, OCO₂R⁷, SO₂R⁷, SO₂NR⁷R⁸ and C(═NR⁷)NR⁷R⁸;
R⁶ is selected from the group consisting of H, C₁₋₆alkyl, C₂₋₆alkenyl, heteroaryl, cycloalkyl, and heterocyclyl;
R⁷ and R⁸ are each independently selected from the group consisting of H, C₁₋₈alkyl, C₂₋₆alkenyl, SO₂C₁₋₆alkyl, (CH₂)ₘ-cycloalkyl, (CH₂)ₘ-aryl, (CH₂)ₘ-heterocyclyl, and (CH₂)ₘ-heteroaryl, wherein m=0, 1 or 2,
or R⁷ and R⁸ together with the nitrogen atom to which they are bound, form a heterocyclyl group; and
wherein any of said alkyl, alkenyl and alkynyl groups may be optionally substituted with up to three members selected from the group consisting of halo, hydroxyl, oxo, cyano, NR⁷R⁸, C₁₋₆alkyl, OC₁₋₆alkyl, S(O)C₁₋₆alkyl, S(O)₂C₁₋₆alkyl and SO₂NR⁷R₈; and
wherein any of said cycloalkyl, heterocyclyl, aryl, and heteroaryl groups may be optionally substituted with up to three substituents selected from the group consisting of C₁₋₆alkyl, C₁₋₆alkoxy, C₁₋₆alkylsulfenyl, C₁₋₆alkylsulfinyl, C₁₋₆alkylsulfonyl, hydroxy, oxo, mercapto, nitro, cyano, halo, C₁₋₆perfluoroalkyl, amino optionally substituted by C₁₋₆alkyl, carbamoyl optionally substituted by C₁₋₆alkyl, NR⁷R⁸, carboxy and aminosulfonyl optionally substituted by C₁₋₆alkyl;
wherein when (R¹)ₐ is located at the 2′ position, (R¹)ₐ is not NR⁶-aryl, NR⁶—C₆H₄NR⁷R⁸, NR⁶—C₆H₄(CH₂)NR⁷R⁸, NR⁷R⁸ where R⁷ or R⁸ is (CH₂)ₘ-aryl and m is O, or N-(aryl)[(C═NR⁷)NR⁷R⁸]; and
wherein when R⁴ is at the C-7 position, R⁴ is not halo, heterocyclyl, aryl, heteroaryl, phthalamido, C₆H₄NR⁷R⁸ or C₆H₄(CH₂)NR⁷R⁸;
or a pharmaceutically acceptable salt, solvate or physiologically functional derivative thereof.

4-5. (canceled)
